(19) United States
(12) Patent Application Publication
(10) Pub. No.: US 2022/0184087 A1
(43) Pub. Date: Jun. 16, 2022

(54) METHODS AND COMPOSITIONS RELATING TO TREATMENT OF CANCER

(71) Applicant: The Penn State Research Foundation, University Park, PA (US)

(72) Inventors: Venkata Saketh Sriram Dinavahi, Hershey, PA (US); Raghavendra Gowda Chandagalu Doreswamy, Plainsboro, NJ (US); Gavin P. Robertson, Hummelstown, PA (US)

(73) Assignee: The Penn State Research Foundation, University Park, PA (US)

(21) Appl. No.: 17/599,188

(22) PCT Filed: Mar. 30, 2020
(86) PCT No.: PCT/US2020/025693

§ 371 Date: Sep. 28, 2021

Related U.S. Application Data
(60) Provisional application No. 62/825,173, filed on Mar. 28, 2019.

(51) Int. Cl. A61K 31/519; A61P 35/00; C07K 16/28

(52) U.S. Cl. CPC A61K 31/519; C07K 16/2818; A61P 35/00

(57) ABSTRACT

According to aspects of the present disclosure, methods and compositions are provided which relate to treating cancer in a subject in need thereof, including: administering 1) an anti-cancer immune therapeutic agent, and 2) a stimulator of p53 levels and/or p53 activity, as a combination formulation or separately, thereby increasing levels and/or activity of p53, and activating NK cell activity in the cancer, providing therapeutic benefits, converting a “cold tumor” into a “hot tumor” leading to a TME responsive to immunotherapy via tumor cell recognition and killing that is not dependent on T-cell mediated immunity.

### REFERENCE TO RELATED APPLICATION

This application claims priority from U.S. Provisional Patent Application Ser. No. 62/825,173, filed Mar. 28, 2019, the entire content of which is incorporated herein by reference.

### FIELD OF THE INVENTION

According to general aspects of the present disclosure, methods and compositions are provided which relate to treating cancer in a subject in need thereof, including: administering 1) an anti-cancer immune therapeutic agent, and 2) a stimulator of p53 levels and/or p53 activity, as a combination formulation or separately, thereby increasing levels and/or activity of p53, and activating NK cell activity in the cancer, providing therapeutic benefits, converting a “cold tumor” into a “hot tumor” leading to a TME responsive to immunotherapy via tumor cell recognition and killing that is not dependent on T-cell mediated immunity.

According to specific aspects of the present disclosure, methods and compositions are provided which relate to treating melanoma in a subject in need thereof, including: administering 1) an anti-cancer immune therapeutic agent, 2) an AKT inhibitor, and 3) a WEE1 inhibitor, as a combination formulation of any 2 or more thereof, or separately, thereby increasing levels and/or activity of p53, and activating NK cell activity in the cancer, providing therapeutic benefits, converting a “cold tumor” into a “hot tumor” leading to a TME responsive to immunotherapy via tumor cell recognition and killing that is not dependent on T-cell mediated immunity.

### BACKGROUND OF THE INVENTION

Incidence and mortality rates for cancers, including malignant melanoma, continue to rise annually. Advanced stage cancer patients, such as advanced stage melanoma patients, remain difficult to treat and metastatic disease is a significant cause of morbidity and mortality.

For example, an estimated 76,000 new cases of melanoma and over 9,000 deaths will occur this year in the United States. Advanced-stage metastatic melanoma carries a poor prognosis, with an overall median survival of 2 to 8 months, and with only 5% of patients surviving beyond 5 years.

Treatment with Vemurafenib (Vem) led to a progression free survival (PFS) of 6.3 months in BRAF^{V600E} mutant melanoma patients without brain metastasis compared to 3.7 months in patients with brain metastasis, see Larkin, J., et al., Eur J Cancer, 2019, 107: p.175-185. Immune checkpoint inhibitors have revolutionized the treatment regimen of melanoma but the median PFS with ipilimumab (anti-CTLA4) was only 2.9 months while it was 6.9 months with nivolumab (anti-PD-1) as reported in Larkin, J., et al.,. N Engl J Med, 2015, 373(1): p.23-34. Combination of these agents had a better response with PFS of 11.5 months with 57% of patients responding to treatment. However, the combination treatment had severe (Grade 3 and 4) adverse reactions in 55% of the patients, see Larkin, J., et al.,. N Engl J Med, 2015, 373(1): p.23-34. Additionally, the response rates fall to 46% and 22% with asymptomatic and symptomatic brain metastases, as reported in Long, G. V., et al., Lancet Oncol, 2018, 19(5): p. 672-681.

Currently, significant efforts are being made to improve long-term management of patients with cancer using with newer drugs and rational strategies for combination-based therapy but there is a continuing need for compositions and methods for treatment of cancer. Further, better approaches are needed for the more effective long-term treatment of advanced-stage cancer patients with metastasis.

### SUMMARY OF THE INVENTION

Methods of treating cancer in a subject in need thereof according to aspects of the present disclosure include administering a combination of: 1) an anti-cancer immune therapeutic agent, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent or a non-cell based agent, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent or a non-cell based agent, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent selected from: an NK cell-based anti-cancer immune therapeutic agent; and a CAR-T cell-based anti-cancer immune therapeutic agent, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent selected from: an NK cell-based anti-cancer immune therapeutic agent; and a CAR-T cell-based anti-cancer immune therapeutic agent, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: a PD-1 inhibitor, a PD-1 ligand (PD-L1) inhibitor, and a CTLA4 inhibitor, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: a PD-1 inhibitor, a PD-1 ligand (PD-L1) inhibitor, and a CTLA4 inhibitor, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR, and 2) a stimulator of p53 levels and/or p53 activity, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR, and 2) a stimulator of p53 levels and/or p53 activity, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, and 2) a stimulator of p53 levels and/or p53 activity which inhibits degradation of p53, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, and 2) a stimulator of p53 levels and/or p53 activity which inhibits degradation of p53, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, and 2) a stimulator of p53 levels and/or p53 activity which is an inhibitor of AKT and/or an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, and 2) a stimulator of p53 levels and/or p53 activity which is an inhibitor of AKT and/or an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent or a non-cell based agent, 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent or a non-cell based agent, 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent or a non-cell based agent, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent or a non-cell based agent, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent selected from: an NK cell-based anti-cancer immune therapeutic agent; and a CAR-T cell-based anti-cancer immune therapeutic agent, 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent selected from: an NK cell-based anti-cancer immune therapeutic agent; and a CAR-T cell-based anti-cancer immune therapeutic agent, 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent selected from: an NK cell-based anti-cancer immune therapeutic agent; and a CAR-T cell-based anti-cancer immune therapeutic agent, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a cell-based agent selected from: an NK cell-based anti-cancer immune therapeutic agent; and a CAR-T cell-based anti-cancer immune therapeutic agent, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor, 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor, 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: a PD-1 inhibitor, a PD-1 ligand (PD-L1) inhibitor, and a CTLA4 inhibitor, 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: a PD-1 inhibitor, a PD-1 ligand (PD-L1) inhibitor, and a CTLA4 inhibitor, 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: a PD-1 inhibitor, a PD-1 ligand (PD-L1) inhibitor, and a CTLA4 inhibitor, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: a PD-1 inhibitor, a PD-1 ligand (PD-L1) inhibitor, and a CTLA4 inhibitor, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, 2) an inhibitor of AKT, and 3) an inhibitor of WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, 2) an inhibitor of AKT, and 3) an inhibitor of WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

Methods of treating cancer in a subject in need thereof according to aspects of the present disclosure include administering a combination of: 1) an anti-cancer immune therapeutic agent which includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, 2) an inhibitor of AKT selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) an inhibitor of WEE1 selected from the group consisting of: MK1775, a pharmaceutically acceptable salt, hydrate, amide or ester thereof, an siRNA directed to WEE1, and a CRISPR/Cas knockdown construct directed to WEE1, as a combination formulation or separately, wherein the cancer is characterized by wild-type p53 expression. According to aspects of a method of treating cancer of the present disclosure, administration of the combination provides a synergistic effect, thereby treating the cancer. According to aspects of a method of treating cancer of the present disclosure, administration of the combination stimulates immunogenic tumor cell death of cancer cells of the cancer. According to aspects of a method of treating cancer of the present disclosure, administration of the combination stimulates recruitment of NK cells to the cancer and/or activates NK cells in the cancer.

According to aspects of a method of treating cancer of the present disclosure, the cancer is characterized by constitutive activation of a mitogen-activated protein kinase-signaling pathway.

According to aspects of a method of treating cancer of the present disclosure, the cancer is characterized by constitutive activation of a mitogen-activated protein kinase-signaling pathway associated with one or more mutations in BRAF, KIT and/or RAS.

According to aspects of a method of treating cancer of the present disclosure, the cancer is characterized by constitutive activation of a mitogen-activated protein kinase-signaling pathway associated with ^{6V00E} BRAF.

According to aspects of a method of treating cancer of the present disclosure, the cancer is characterized by AKT dysregulation.

According to aspects of a method of treating cancer of the present disclosure, the cancer is selected from the group consisting of: melanoma, colorectal cancer, thyroid cancer, breast cancer, prostate cancer, sarcoma, glioblastoma, T-cell acute lymphoblastic leukaemia, lung cancer and liver cancer.

According to aspects of a method of treating cancer of the present disclosure, the cancer is melanoma.

According to aspects of a method of treating cancer of the present disclosure, a method of treating cancer further includes: obtaining a first sample containing or suspected of containing cancer cells from the subject prior to administering the combination of the anti-cancer immune therapeutic agent and the stimulator of p53 levels and/or activity; obtaining a second sample containing or suspected of containing cancer cells from the subject after administering the combination; and assaying the first and second samples for one or more markers of apoptosis, thereby monitoring effectiveness of administering the combination.

According to aspects of a method of treating cancer of the present disclosure, a method of treating cancer further includes: obtaining a first sample containing or suspected of containing cancer cells from the subject prior to administering the combination of the anti-cancer immune therapeutic agent and the stimulator of p53 levels and/or activity; obtaining a second sample containing or suspected of containing cancer cells from the subject after administering the combination; and assaying the first and second samples for activity of a mitogen-activated protein kinase-signaling pathway, thereby monitoring effectiveness of administering the combination.

According to aspects of a method of treating cancer of the present disclosure, a method of treating cancer further includes: obtaining a first sample containing or suspected of containing cancer cells from the subject prior to administering the combination of the combination of the anti-cancer immune therapeutic agent and the stimulator of p53 levels and/or activity; obtaining a second sample containing or suspected of containing cancer cells from the subject after administering the combination; and assaying the first and second samples for AKT dysregulation, thereby monitoring effectiveness of administering the combination.

According to aspects of a method of treating cancer of the present disclosure, a method of treating cancer further includes: obtaining a first sample containing or suspected of containing cancer cells from the subject prior to administering the combination of the anti-cancer immune therapeutic agent and the stimulator of p53 levels and/or activity; obtaining a second sample containing or suspected of containing cancer cells from the subject after administering the combination; and assaying the first and second samples for p53 expression and/or an associated gene selected from the group consisting of: CLCA2, PVRL4, SULF2, CDKN1a, BTG2, ACTA2, TP5363, FDXR, GDF15, IGFBP5 and ADAM19, wherein an increase in p53 expression and/or expression of an associated gene selected from the group consisting of: CLCA2, PVRL4, SULF2, CDKN1a, BTG2, ACTA2, TP5363, FDXR, GDF15, IGFBP5 and ADAM19, is an indicator of an anti-cancer cell effect of treatment with the combination, thereby monitoring effectiveness of administering the combination.

According to aspects of a method of treating cancer of the present disclosure, a method of treating cancer further includes: obtaining a first sample containing or suspected of containing cancer cells from the subject prior to administering the combination of the anti-cancer immune therapeutic agent and the stimulator of p53 levels and/or activity; obtaining a second sample containing or suspected of containing cancer cells from the subject after administering the combination; and assaying the first and second samples for FOXM1 expression and/or expression of an associated gene selected from the group consisting of: TMPO, ANP32E, SMC4, KIF20B, ASPM, DEPDC1, NCAPG, CENPE, wherein a decrease in expression of FOXM1 and/or an associated gene selected from the group consisting of: TMPO, ANP32E, SMC4, KIF20B, ASPM, DEPDC1, NCAPG and CENPE, is an indicator of an anti-cancer cell effect of treatment with the combination, thereby monitoring effectiveness of administering the combination.

According to aspects of a method of treating cancer of the present disclosure, the anti-cancer immune therapeutic agent and the stimulator of p53 levels and/or activity are administered simultaneously.

According to aspects of a method of treating cancer of the present disclosure, all of: the anti-cancer immune therapeutic agent and the stimulator of p53 levels, are administered sequentially.

According to aspects of a method of treating cancer of the present disclosure, the stimulator of p53 levels and/or p53 activity is an AKT inhibitor and a WEE1 inhibitor, wherein: 1) the anti-cancer immune therapeutic agent and the AKT inhibitor are administered simultaneously and the WEE1 inhibitor is administered sequentially with respect to the anti-cancer immune therapeutic agent and the AKT inhibitor; 2) the anti-cancer immune therapeutic agent and the WEE1 inhibitor are administered simultaneously and the AKT inhibitor is administered sequentially with respect to the anti-cancer immune therapeutic agent and the WEE1 inhibitor; or 3) the WEE1 inhibitor and the AKT inhibitor are administered simultaneously and the anti-cancer immune therapeutic agent is administered sequentially with respect to the WEE1 inhibitor and the AKT inhibitor.

According to aspects of a method of treating cancer of the present disclosure, all of: the anti-cancer immune therapeutic agent, the AKT inhibitor, and the WEE1 inhibitor, are administered sequentially.

According to aspects of a method of treating cancer of the present disclosure, the stimulator of p53 levels and/or p53 activity is an MDM2 or MDM4 inhibitor, and wherein: 1) the anti-cancer immune therapeutic agent and the MDM2 or MDM4 inhibitor are administered simultaneously; or 2) the anti-cancer immune therapeutic agent and the MDM2 or MDM4 inhibitor are administered sequentially.

According to aspects of a method of treating cancer of the present disclosure, the phrase “administered sequentially” refers to administration within a period of time selected from: one hour, two hours, four hours, eight hours, twelve hours, twenty-four hours, 2 days, 3 days, 4 days, 5 days, 6 days and 7 days.

According to aspects of a method of treating cancer of the present disclosure, the subject is human.

According to aspects of a method of treating cancer of the present disclosure, p53 is increased in the subject following administration of the combination.

According to aspects of a method of treating cancer of the present disclosure, the AKT inhibitor is an AKT3 inhibitor.

### BRIEF DESCRIPTION OF THE DRAWINGS

FIG. 1 shows graphs illustrating overexpression of MDM pathways (MDM2 and MDM4) and wild-type p53 status in cutaneous melanomas;

FIG. 2A is a schematic diagram showing interactions of MDM2 and MDM4 with p53, decreased transcriptional activation of proapoptotic genes, decreased immunogenic cell death, and lack of recruitment and/or activation of NK cells in tumor cells, such as melanoma cells;

FIG. 2B is a schematic diagram showing effect of inhibition of MDM2 and MDM4 interactions, such as by inhibition of AKT and Wee1, on p53, increased transcriptional activation of proapoptotic genes, increased immunogenic cell death, and increased recruitment and/or activation of NK cells in tumor cells, such as melanoma cells;

FIG. 3A is an image of a Western blot showing decreased p53 protein due to treatment of UACC 903 human melanoma cell line cells with siRNA against p53;

FIG. 3B shows graphs illustrating that AKT/WEE1 inhibitory efficacy in UACC 903 cells depends on p53 being present in melanoma cells, cells treated with non-specific control scrambled siRNA has no effect on inhibitory efficacy of AKT/WEE1; results represent 3 separate experiments and Calcusyn data analysis;

FIG. 3C shows graphs illustrating that AKT/WEE1 inhibitory efficacy in UACC 903 cells depends on p53 being present in melanoma cells, cells treated anti-p53 siRNA inhibits the effect of AKT/WEE1 inhibitors; results represent 3 separate experiments and Calcusyn data analysis;

FIG. 4 is a graph showing that calreticulin levels increase with AKT/WEE1 inhibition in B16.F10 cells indicating ICD; these results represent one of 2 separate experiments showing statistically significant results;

FIG. 5 is a graph showing that inhibition of AKT/WEE1 inhibits melanoma tumor growth without toxicity; these results represent 2 separate experiments, with an N=8 for each group; ** p<0.01; ***p<0.001;

FIG. 6A is a graph showing that AKT/WEE1/anti-PD-1 treatment of immune system competent mice inhibits tumor development; these results represent 2 separate experiments with an N=13-15 for each group;

FIG. 6B is a graph showing that AKT/WEE1/anti-PD-1 treatment of immune system competent mice increases animal survival; these results represent 2 separate experiments with an N=13-15 for each group;

FIG. 7A is a graph showing increased tumor infiltration of NK cells with AKT/WEE1/anti-PD-1 treatment; these results are representative of one of two similar separate experiments;

FIG. 7B is a graph showing increased tumor infiltration of macrophages with AKT/WEE1/anti-PD-1 treatment; these results are representative of one of two similar separate experiments;

FIG. 7C is a graph showing increased tumor infiltration of dendritic cells with AKT/WEE1/anti-PD-1 treatment; these results are representative of one of two similar separate experiments;

FIG. 8A is a graph showing increased tumor infiltration of NK T-cells with AKT/WEE1/anti-PD-1 treatment; these results are representative of one of two similar separate experiments;

FIG. 8B is a graph showing CD-8 cells with AKT/WEE1/anti-PD-1 treatment; these results are representative of one of two similar separate experiments;

FIG. 8C is a graph showing CD4 cells with AKT/WEE1/anti-PD-1 treatment; these results are representative of one of two similar separate experiments;

FIG. 8D is a graph showing Treg cells with AKT/WEE1/anti-PD-1 treatment; these results are representative of one of two similar separate experiments;

FIG. 9 is a graph showing that NK cell activity increases with AKT/WEE1/anti-PD-1 treatment; these results are representative of one of two similar separate experiments;

FIG. 10A is a graph showing PD-1 activation status of TILs—NK cells—with AKT/WEE1/anti-PD-1 treatment; these results represent one of two similar separate experiments;

FIG. 10B is a graph showing PD-1 activation status of TILs—macrophages—with AKT/WEE1/anti-PD-1 treatment; these results represent one of two similar separate experiments;

FIG. 10C is a graph showing PD-1 activation status of TILs—dendritic cells—with AKT/WEE1/anti-PD-1 treatment; these results represent one of two similar separate experiments; and

FIG. 11 is an image showing immunohistochemistry of tumors indicates a surge in NK cell infiltration; these results are representative of one of two similar separate experiments.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; CRISPR/Cas: A Laboratory Manual, Doudna and Mali (eds), Cold Spring Harbor Laboratory Press, Cold Spring Harbor, N.Y., USA, 2016; Chu, E. and Devita, V. T., Eds., Physicians' Cancer Chemotherapy Drug Manual, Jones & Bartlett Publishers, 2005; J. M. Kirkwood et al., Eds., Current Cancer Therapeutics, 4th Ed., Current Medicine Group, 2001; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

The term “expression,” and grammatical equivalents, refers to transcription of a gene to produce a corresponding mRNA and/or translation of the mRNA to produce the corresponding protein.

Functional inactivation of p53 pathways in cancer cells results in reduced responsiveness to DNA damage, decreased expression of proapoptotic proteins, and impaired anti-tumor immunity, leading to the creation of an immune suppressive tumor microenvironment (TME).

Many cancers, including eighty percent of advanced stage melanoma patients, have high levels of Murine Double Minute (MDM) proteins which functionally inactivate p53, resulting in reduced responsiveness to DNA damage, decreased expression of proapoptotic proteins, and impaired anti-tumor immunity, and leading to the creation of an immune suppressive tumor microenvironment (TME). Targeting and thereby inhibiting MDM protein signaling in a subject according to aspects of the present disclosure restores these p53-mediated activities. Inhibiting MDM pathway signaling in cancer, such as melanoma, by inhibiting the AKT and WEE1 pathways increases p53 activity, triggers ICD and uniquely skews the TME towards an inflammatory state more permissive to immunotherapy.

According to particular aspects of the disclosure, the interactions of the MDM proteins with other pathways characteristically dysregulated in cancers, such as melanoma, are targeted to alter the tumor microenvironment (TME) to treat cancers, including metastatic disease. According to particular aspects of the disclosure, MDM protein interaction is modulated by targeting the AKT and WEE1 pathways to synergistically activate p53 signaling, triggering immunogenic cell death (ICD) and activating natural killer (NK)-cell mediated anti-tumor immunity. As described in the present disclosure, inhibiting MDM signaling in cancer, such as melanoma, increases levels and/or activity of p53, and activates NK cell activity, providing therapeutic benefits, converting a “cold tumor” into a “hot tumor” leading to a TME responsive to immunotherapy via tumor cell recognition and killing that is not dependent on T-cell mediated immunity.

According to aspects of methods of treatment according to the present disclosure, the TME in tumors, such as metastatic melanoma lesions, is primed by inhibiting deregulated MDM pathways in a tumor to turn “cold” immune cell-poor tumors into “hot” NK cell-rich tumors making the tumor more responsive to immunotherapy.

Inhibiting MDM signaling in tumors, such as melanoma, alters immunogenicity to specifically activate natural killer (NK) cell anti-tumor immunity. The present disclosure demonstrates that disrupting deregulated MDM protein signaling intumors, such as melanoma, upregulates p53 activity and significantly alters the TME, thereby providing a TME that is more responsive to immunotherapy.

According to aspects of methods of treatment according to the present disclosure, this is accomplished by targeting MDM pathway signaling by inhibiting the AKT and WEE1 pathways to increase proapoptotic protein production in tumor cells to trigger immunogenic cell death (ICD) and increase recruitment of immune cells into the TME, in combination with inhibition of an anti-cancer immune therapeutic agent, such as inhibiting PD-1 using an anti-PD-1 agent. Inhibition of WEE1 restores sensitivity to DNA damage causing dissociation of the MDM2/4 complex, and enabling transcription of proapoptotic proteins, which promote ICD, while inhibition of AKT prevents p53 ubiquitination increasing p53 protein levels. Thus, methods of treatment according to the present disclosure synergistically enhance tumor inflammation, largely mediated by NK cell recruitment and activation, increasing the efficacy of immunotherapy.

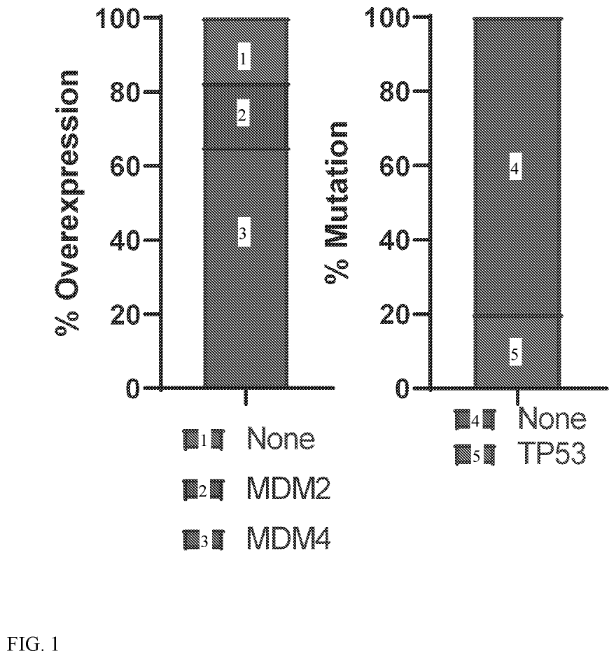

FIG. 1 shows aspects of deregulated signaling in cancers, particularly melanoma since 80% of melanoma patients have deregulated MDM signaling. 80-90% of melanoma patient tumors express wildtype p53 (see for example, Chin, L. et al., Genes Dev, 2006, 20(16): p. 2149-82 and Zhang, T. et al., Pigment Cell Melanoma Res, 2016, 29(3): p. 266-83); however, p53 is often inactivated by increased expression of MDM2/4 (see for example Lee, B. et al, Curr Opin Oncol, 2015, 27(2): p. 141-50; Wu, C. E. et al, Br J Cancer, 2018, 118(4): p. 495-508; and de Polo, A. et al, J Mol Cell Biol, 2017, 9(2): p. 154-165). Elevated MDM2 levels increase p53 protein ubiquitination and degradation as detailed in Fan, C. et al, Cell Cycle, 2017, 16(7): p. 660-664; and Brooks, C. L. et al, Mol Cell, 2006, 21(3): p. 307-15. MDM4 upregulation promotes cell survival by antagonizing p53 proapoptotic function, see for example Lu, M. et al., FEBS Lett, 2014, 588(16): p. 2616-21; and Gembarska, A., et al., Nat Med, 2012, 18(8): p. 1239-47.

AKT and WEE1 levels are high in melanoma cells and are important pathways that promote melanoma cell survival and metastasis. Those cancers having wildtype p53 tend to have high MDM protein levels which functionally inactivate the pathway.

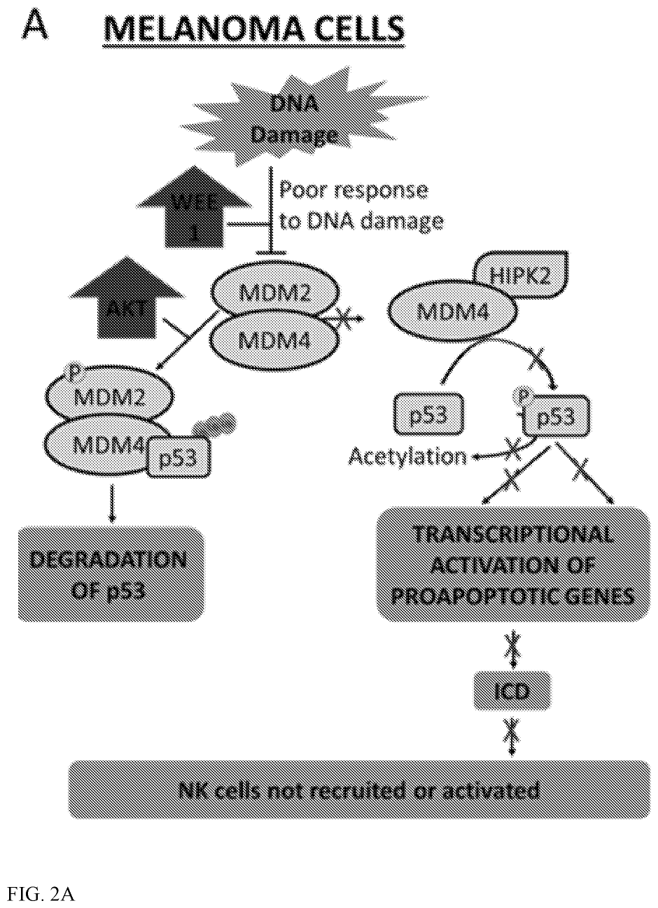

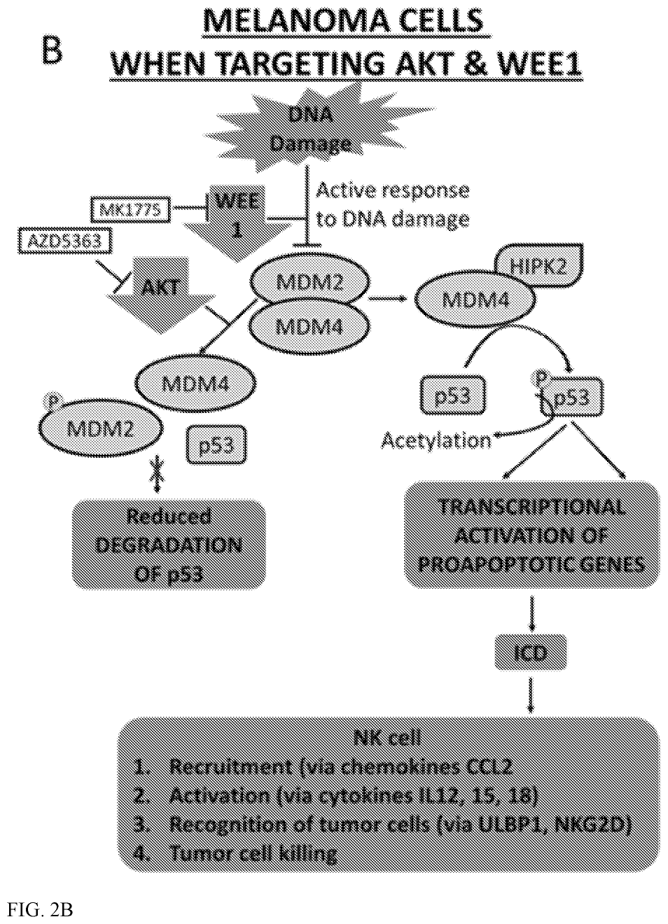

FIG. 2A illustrates that high WEE1 levels make melanoma cells less sensitive to DNA damage. As a result, the MDM2/4 complex does not easily disassociate and is phosphorylated by high AKT activity promoting binding to p53, which leads to p53 ubiquitination and degradation. Furthermore, decreased MDM2/4 dissociation leads to a lack of free MDM4, which limits interaction with HIPK2, reducing phosphorylation of p53 at Ser46. Phosphorylation is needed for p53 acetylation, which controls transcriptional activation of proapoptotic targets and induction of ICD.

FIG. 2B illustrates that these defects can be corrected in cancers, such as advanced primary melanoma lesions and disseminated metastases, by inhibiting AKT and WEE1 using methods of the present disclosure. Specifically, inhibiting WEE1 increases sensitivity of tumor cells to DNA damage leading to the release of MDM4 from the MDM2/4 complex making it available for interaction with HIPK2, promoting phosphorylation and subsequent acetylation of p53. Furthermore inhibiting AKT regulates AKT-mediated phosphorylation of the MDM2/4 complex, reducing ubiquitination and degradation of p53. The resultant enhancement of proapoptotic genes inhibits tumor development by triggering ICD, NK cell activation, tumor inflammation and anti-tumor immunity.

Established solid tumors usually have poor NK-cell infiltration due to limited homing to the tumor and an immunosuppressive microenvironment. However, intra-tumoral NK-cell numbers correlate with good prognosis and/or anti-metastatic effects in tumors, such as lung, head and neck, gastric and colorectal carcinomas. NK cell and conventional type 1 dendritic cell infiltration in melanoma can predict response to immunotherapy, such as administration of an anti-PD-1 agent, in both pre-clinical and clinical settings. Therefore, therapeutic approaches to effectively harness NK cell activity against tumors, such as melanoma, are needed.

Methods of treatment according to aspects of the present disclosure restore p53 pathway functionality by inhibiting AKT and WEE1, and which activates NK cell anti-tumor immunity, to treat tumors, such as advanced-stage melanoma.

Methods of treatment according to aspects of the present disclosure increase the number and/or activity of NK cells in a tumor, providing NK cell-mediated anti-tumor immune activity in the tumor, such as melanoma, providing immune activity distinct from T-cell CD8 mediated immunity.

Compositions and methods according to aspects of the present disclosure relate to a combination of: 1) an anti-cancer immune therapeutic agent, and 2) a stimulator of p53 levels and/or p53 activity, as a combination formulation or separately, for treatment of cancer in a subject.

Anti-Cancer Immune Therapeutic Agents

The term “anti-cancer immune therapeutic agent” as used herein refers to agents which activate or suppress a component of the immune system of a subject for treatment of cancer in the subject. An anti-cancer immune therapeutic agent can be a cell-based agent, such as natural killer cells (NK cells), cytotoxic T lymphocytes, lymphocytes, macrophages, dendritic cells, and the like. An “anti-cancer immune therapeutic agent” which is a cell-based agent can include modified cells, such as genetically-modified, chemically-modified, or biochemically-modified, immune cells. Alternatively, “an anti-cancer immune therapeutic agent” can be a small molecule, protein (such as, but not limited to, an antibody), peptide, saccharide, nucleic acid, or other non-cell based agent. An “anti-cancer immune therapeutic agent” can be an immune checkpoint inhibitor.

NK Cell-Based Anti-Cancer Immune Therapeutic Agents

Natural killer (NK) cells are a critical component of the innate immune response against malignant cells. They were identified by their ability to kill tumor cells without prior sensitization to tumor antigens. This is distinct from the mechanism by which T-cells lyse tumor cells, which requires recognition of tumor antigens presented in the context of major histocompatibility class I or II by a specific T-cell receptor. Due to the delay in priming and expansion of T-cells bearing a particular tumor antigen specific receptor, NK cells act as a first line of defense against newly transformed cells. Thus, Natural killer (NK) cells are immunotherapeutic agents in particular in the fight against cancers.

Non-limiting examples of NK cell-based anti-cancer immune therapeutic agents include autologous NK cells, ex-vivo stimulated mblL-21 allogeneic NK, ex vivo expanded allogeneic NK cells, and NK-92 (Neukoplast).

CAR-T Cell-Based Anti-Cancer Immune Therapeutic Agents

Chimeric antigen receptor T cells (also known as CAR T cells) are T cells that have been genetically engineered to produce an artificial T-cell receptor. Chimeric antigen receptors (CARs, also known as chimeric immunoreceptors, chimeric T cell receptors or artificial T cell receptors) are receptor proteins that have been engineered to give T cells the new ability to target a specific protein. The receptors are chimeric because they combine both antigen-binding and T-cell activating functions into a single receptor. CAR-T cell therapy uses T cells engineered with CARs for cancer therapy. The premise of CAR-T immunotherapy is to modify T cells to recognize cancer cells in order to more effectively target and destroy them.

Non-Cell Based Anti-Cancer Immune Therapeutic Agents

Particular non-cell based anti-cancer immune therapeutic agents include, but are not limited to, indoleamine 2,3-dioxygenase 1 (IDO1) inhibitors, lymphocyte-activation gene 3 (LAG3) antibodies, T-cell immunoglobulin and mucin domain-3 (TIM3) antibodies, OX-40 agonists, Glucocorticoid-induced TNFR-related (GITR), and immune checkpoint inhibitors.

IDO1 inhibitors include, but are not limited to, indoximod, navoximod, epacadostat, INCB024360, BMS-986205.

LAG3 antibodies include, but are not limited to, BMS-986016, LAG525, MK-4280, GSK2831781, IMP321.

TIM3 antibodies include, but are not limited to, MBG453, TSR-022, LY3321367.

OX-40 agonists include, but are not limited to, OX86, Fc-OX4OL, MOXR0916 and GSK3174998.

GITR include, but are not limited to, TRX518, MK-4166, MK-1248, AMG 228, BMS-986156, INCAGN01876, MEDI1873, GWN323.

Immune Checkpoint Inhibitors

The term “immune checkpoint inhibitor” refers to a substance having activity to specifically inhibit immune checkpoint activity, including inhibition of T cells expressing PD-1 to suppress immune reaction to cancer cells.

Immune checkpoint inhibitors include, but are not limited to, PD-1 inhibitors, PD-L1 inhibitors, and CTLA4 inhibitiors.

An immune checkpoint inhibitor can be a PD-1 inhibitor, a PD-1 ligand (PD-L1) inhibitor, or a CTLA4 inhibitor.

PD-L1 inhibitors are anti-PD-L1 antibodies such as, but not limited to, atezolizumab, avelumab, and durvalumab; and an antigen-binding fragment of any one of the foregoing. siRNA or a CRISPR/Cas knockdown construct directed to PD-L1 can be used to inhibit PD-L1.

CTLA4 inhibitors are anti-CTLA4 antibodies such as, but not limited to ipilimumab and an antigen-binding fragment thereof. siRNA or a CRISPR/Cas knockdown construct directed to CTLA4 can be used to inhibit CTLA4.

PD-1 (programmed cell death protein 1, also known as CD279) is a cell surface receptor protein present on T cells. PD-1 interacts with a ligand, PD-L1 or PD-L2, regulating T cell activation and tolerance. The interaction of PD-1 with its ligand provides a negative signal which protects tissues from immune system attack. Further, interaction of PD-1 with its ligand provides a negative signal can protect cancer cells from immune system attack; see for example, Jin et al., Current Topics in Microbiol. Immunol., 350:17-37, 2011. PD-1 is often overexpressed in various cancers. The structure and function of PD-1 has been described, along with inhibitors of PD-1, see for example, Zhan et al., Drug Discovery Today, 21:1027-1036, 2016; Zak et al., Structure, 23:2341-2348, 2015; and Zak et al., Structure, 25:1163-1174, 2017.

The term “PD-1 inhibitor” refers to a substance having activity to specifically inhibit PD-1 activity. Inhibition of PD-1 activity includes inhibition of T cells expressing PD-1 to suppress immune reaction to cancer cells. A PD-1 inhibitor inhibits PD-1 suppression of immune reaction to cancer cells in melanoma and other cancers and thereby inhibits cancer cell survival and proliferation. A PD-1 inhibitor can be an agent effective to interaction of a PD-1 ligand with PD-1, thereby inhibiting PD-1 activity. An PD-1 inhibitor reduces PD-1 activity by at least 10%, 20%, 30%, 40%, 50%, 60%, 70%, 80%, 90%, 95%, 99% or more, compared to a control.

PD-1 inhibitors include, but are not limited to, anti-PD-1 antibodies, small molecule PD-1 inhibitors, and SiRNA or a CRISPR/Cas knockdown construct directed to PD-1. Anti-PD-1 antibodies include, but are not limited to, nivolumab and pembrolizumab; and an antigen-binding fragment of any one of the foregoing. Cemiplimab is an anti-PD-1 antibody and an antigen-binding fragment of cemiplimab can be used. Pidilizumab (and antigen binding fragments thereof), AMP-224, AMP-514, and PDR001 are PD-1 inhibitors. PD-1 inhibitors can be obtained commercially or chemically synthesized according to known methods.

Stimulator of p53

The phrase “a stimulator of p53 levels and/or activity” refers to a a substance having activity to specifically increase p53 levels and/or activity, including inhibition of one or more Murine Double Minute (MDM) proteins and/or inhibiting signaling pathways mediated by MDM proteins, such as inhibiting the AKT and WEE1 signaling pathways, responsible for functional inactivation of p53 in cancer cells, such as, but not limited to, melanoma cells.

MDM Inhibitor

Mouse double minute 2 homolog (MDM2) also known as E3 ubiquitin-protein ligase. MDM2 is a protein that in humans is encoded by the MDM2 gene. MDM2 is a negative regulator of the p53 tumor suppressor protein. MDM2 protein binds to andubiquitinates the p53 tumor suppressor protein, resulting in degradation of p53, and thereby inhibiting p53 transcriptional activation. Mouse double minute 4 homolog (MDM4) encodes MDM4 which is a negative regulator of p53, binding to p53 and antagonizing its proapoptotic function.

Compositions and methods according to aspects of the present disclosure relate to inhibition of murine double minute (MDM) proteins, such as MDM2 and MDM4, which reduce the transcriptional transactivation of proapototic proteins in tumor cells and promote an immune suppressive tumor microenvironment, for treatment of cancer in a subject.

Compositions and methods according to aspects of the present disclosure relate to inhibition of murine double minute (MDM) proteins, such as MDM2 and MDM4, to induce p53, for treatment of cancer in a subject.

Compositions and methods according to aspects of the present disclosure relate to inhibition of MDM2 and/or MDM4 by administration of one or more of: RG7112, RG7388, SAR405838, MK-8242, AMG232, DS-3032b, HDM201, CGM097, ALRN-6924, Nutlin-3, SJ172550, NSC207895, to increase levels and/or activity of p53, for treatment of cancer characterized by wild-type p53 in a subject.

The term “MDM inhibitor” refers to a substance having activity to specifically inhibit MDM activity in a cell in vitro or in vivo. Inhibition of MDM activity includes inhibition of one or both of MDM2 and MDM4. An MDM inhibitor inhibits MDM activity abnormally activated in melanoma and other cancers and inhibits cancer cell survival and proliferation. An MDM inhibitor can be an agent effective to reduce the expression of one or both of MDM2 and MDM4 in a cancer cell, thereby inhibiting MDM activity and promoting p53 activity. An MDM inhibitor reduces MDM activity in a cell by at least 10%, 20%, 30%, 40%, 50%, 60%, 70%, 80%, 90%, 95%, 99% or more, compared to a control.

Inhibitors of MDM2 and/or MDM4 include NSC66811, SP141, RG7112, RG7388, SAR405838, MK-8242, AMG232, DS-3032b, HDM201, CGM097, ALRN-6924, Nutlin-3, SJ172550, NSC207895 anti-MDM antibodies such as such as anti-MDM2 or anti-MDM4 antibodies; anti-MDM peptides; and anti-MDM nucleic acids such as anti-MDM2 or anti-MDM4 siRNA, all of which can be obtained commercially or chemically synthesized according to known methods, see Tisato, V., et al., J Hematol Oncol, 2017, 10(1): p.133.

SJ-172550 which binds with high affinity to MDM4 within its p53-binding pocket and disrupts the p53-MDM4 and MDM4-HIPK2 complexes (Mancini, F., et al., Oncogene, 2016, 35(2): p. 228-40). Similarly, nutlin-3 inhibits the p53-MDM2 and p53-MDM2-MDM4 complexes (Mancini, F., et al., Oncogene, 2016, 35(2): p. 228-40). Co-immunoprecipitation is used to demonstrate that inhibition of AKT/WEE1 alters the MDM2/p53, MDM2/MDM4/p53, MDM4/p53, or MDM4/HIPK2 complexes.

MDM inhibitors include siRNA directed to MDM effective to decrease MDM protein in a cell containing the siRNA or CRISPR composition directed to MDM.

According to aspects of the present disclosure, an siRNA or CRISPR composition directed to MDM is directed to MDM2 and/or MDM4.

AKT Inhibitor

AKT is a serine/threonine protein kinase, also known as protein kinase B, which has a stimulatory effect on cell cycle progression, cell proliferation and inhibition of apoptosis. AKT proteins, nucleic acids and signaling pathway components are described, for instance, see Testa, J. R. et al., PNAS, 98:10983-10985; Fayard, E. et al., J. Cell Sci., 118:5675-5678, 2005; Cheng, J. and S. Nicosia, (2001) AKT signal transduction pathway in oncogenesis, in Encyclopedic Reference of Cancer, D. Schwab, Editor. 2001, Springer: Berlin, Germany, p. 35-7; Datta, S. R., et al. (1999) Cellular survival: a play in three AKTs. Genes Dev, 13(22): 2905-27; Fayard, E. et al. (2005) J Cell Sci, 118(Pt 24: 5675-8; Mirza, A. M., Fayard, E. et al. (2000) 2000. 11(6: 279-92; Nicholson, K. M. and N. G. Anderson, (2002)Cell Signal, 2002, 14(5): p. 381-95; Paez, J. and W. Sellers, (2003) P13K/PTEN/AKT Pathway: A Critical Mediator of Oncogenic Signaling, in Signal Transduction in Cancer, D. Frank, Editor. 2003, Kluwer Academic Publishers: Netherlands; and Testa, J. R.; P. N. Tsichlis, (2005) Oncogene, 24(50): 7391-3 and other references listed herein.

AKT family members, AKT1, AKT2 and AKT3, are activated by phosphorylation, membrane translocation, increases in gene copy number and/or loss of a negative regulatory phosphatase, PTEN. Increased activation of AKT, including increased levels of AKT and/or increased levels of phosphorylated AKT is an indicator of AKT dysregulation associated with proliferation and cell survival in pathogenic conditions, such as cancer.

AKT 3 is active in ˜70% of melanomas. While all three AKT isoforms are expressed in melanocytes and melanoma cells, AKT3 is the predominantly active family member. Dysregulated AKT3 activity in melanoma cells reduces cellular apoptosis mediated through caspase-3, thereby promoting melanoma tumor development. As a well-established survival factor, hyperactivation of the AKT pathway is observed in many types of cancers, as described in Altomare et al, Oncogene 2005; 24(50): 7455-64.

AKT dysregulation is determined, for instance, by measurement of AKT gene copy number, AKT protein or RNA levels and/or levels of phosphorylated AKT, in cells known or suspected to be dysplastic, pre-cancerous, cancerous, metastatic or otherwise characterized by abnormal cell proliferation compared to normal cells. Assays for AKT dysregulation include, but are not limited to, immunoassays and nucleic acid assays.

The term “AKT inhibitor” refers to a substance having activity to specifically inhibit AKT activity in a cell in vitro or in vivo. Inhibition of AKT activity includes inhibition of all or one or more of AKT1, AKT2 and AKT3. An AKT inhibitor inhibits AKT activity abnormally activated in melanoma and other cancers and inhibits cancer cell survival and proliferation. An AKT inhibitor can be an agent effective to reduce the expression of all or one or more of AKT1, AKT2 and AKT3 in a cell, thereby inhibiting AKT activity. An AKT inhibitor reduces AKT activity in a cell by at least 10%, 20%, 30%, 40%, 50%, 60%, 70%, 80%, 90%, 95%, 99% or more, compared to a control.

AKT inhibitors include, but are not limited to, GDC0068 (commercially available from suppliers such as Chemie Tek, Indianapolis, Ind.) also known as GDC-0068, ipatasertib and RG7440; MK-2206, MK-2206.2HCl; perifosine (also known as KRX-0401); GSK690693; AT7867; triciribine; CCT128930; A-674563; PHT-427; Akti-1/2; afuresertib (also known as GSK2110183); AT13148; GSK2141795; BAY1125976; uprosertib (aka GSK2141795); AZD5363 also known as AZD-5363 (commercially available from suppliers such as Chemie Tek, Indianapolis, Ind.); anti-AKT antibodies; anti-AKT peptides; and anti-AKT nucleic acids such as anti-AKT siRNA, all of which can be obtained commercially or chemically synthesized according to known methods.

AKT inhibitors include siRNA directed to AKT effective to decrease AKT protein in a cell containing the siRNA or CRISPR composition directed to AKT.

According to aspects of the present disclosure, an siRNA or CRISPR composition directed to AKT is directed to AKT3.

According to aspects of the present disclosure, inhibition of AKT activity includes inhibition one or more of human AKT1, AKT2 and AKT3.

According to aspects of the present disclosure, the AKT inhibitor is an AKT3 inhibitor.

According to aspects of the present disclosure, the AKT inhibitor is an inhibitor of human AKT3.

WEE1 Inhibitor

The term “WEE1” as used herein refers to a protein kinase, particularly a human protein kinase. WEE1 is involved in the regulation of cell cycle by phosphorylating and inactivating cyclin-dependent kinase-1 (CDK1), see Watanabe et al., 1995, EMBO, 14:1878-1891.

As a component of G2/M checkpoint, WEE1 determines the time point for entry into mitosis and inhibits early progression of cell cycle. WEE1 is also involved in the coordination of cellular response to DNA damage. Furthermore, WEE1 was also identified as a key signaling molecule lying downstream of ^{V600E}BRAF in the MAPK signaling cascade, see Sharma et al., 2013, Am. J. Pathol., 182:1151-1162.

The term “WEE1 inhibitor” refers to a substance having activity to specifically inhibit WEE1 activity in a cell in vitro or in vivo. Inhibition of WEE1 activity includes inhibition one or both of WEE1A and WEE1B. A WEE1 inhibitor inhibits WEE1 activity abnormally activated in melanoma and other cancers and inhibits cancer cell survival and proliferation. A WEE1 inhibitor can be an agent effective to reduce the expression of all or one or both of WEE1A and WEE1B in a cell, thereby inhibiting WEE1 activity. A WEE1 inhibitor reduces WEE1 activity in a cell by at least 10%, 20%, 30%, 40%, 50%, 60%, 70%, 80%, 90%, 95%, 99% or more, compared to a control.

According to aspects of the present disclosure, inhibition of WEE1 activity includes inhibition one or both of human WEE1A and WEE1B.

According to aspects of the present disclosure, the WEE1 inhibitor is a WEE1A inhibitor.

WEE1 inhibitors include, but are not limited to, MK1775 (commercially available from suppliers such as Chemie Tek, Indianapolis, Ind.), also known as MK-1775, AZD1775 and 2-allyl-1-(6-(2-hydroxypropan-2-yl)pyridin-2-yl)-6-((4-(4-methylpiperazin-1-yl)phenyl)amino)-1H-pyrazolo[3,4-d]pyrimidin-3(2H)-one; MK-3652; PD-166285 and PD407824, see Matheson, C. J. et al., Trends Pharmacol Sci, 2016. 37(10): p. 872-881; PF-00120130; anti-WEE1 antibodies; anti-WEE1 peptides; and anti-WEE1 nucleic acids such as anti-WEE1 siRNA, all of which can be obtained commercially or chemically synthesized according to known methods.

WEE1 inhibitors include siRNA or a CRISPR/Cas knockdown construct directed to WEE1 effective to decrease WEE1 protein in a cell containing the siRNA or CRISPR composition directed to WEE1.

According to aspects of the present disclosure, an siRNA or CRISPR composition directed to WEE1 is directed to WEE1A.

Antibody Inhibitors

An anti-cancer immune therapeutic agent and/or a stimulator of p53 levels and/or activity can be an antibody.

As used herein, the terms “antibody” and “antibodies” relate to monoclonal antibodies, polyclonal antibodies, bispecific antibodies, multispecific antibodies, human antibodies, humanized antibodies, chimeric antibodies, camelized antibodies, single domain antibodies, single-chain Fvs (scFv), single chain antibodies, disulfide-linked Fvs (sdFv), and anti-idiotypic (anti-Id) antibodies (including, e.g., anti-Id antibodies to antibodies of the disclosure), and epitope-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG1, IgG2a, IgG2b, IgG2, IgG3, IgG4, IgA1, and IgA2), or subclass.

Examples of antibody fragments that can be used further include Fab fragments, Fab′ fragments, F(ab′)2 fragments, Fd fragments, Fv fragments, scFv fragments, and domain antibodies (dAb). Antibody fragments may be generated by any technique known to one of skill in the art. For example, Fab and F(ab′)2 fragments may be produced by proteolytic cleavage of immunoglobulin molecules, using enzymes such as papain (to produce Fab fragments) or pepsin (to produce F(ab′) 2 fragments). F(ab′) 2 fragments contain the complete light chain, and the variable region, the CH 1 region and the hinge region of the heavy chain. Antibody fragments are also produced by recombinant DNA technologies. Antibody fragments may be one or more complementarity determining regions (CDRs) of antibodies.

An anti-cancer immune therapeutic agent and/or a stimulator of p53 levels and/or activity such as, but not limited to, an immune checkpoint inhibitor, an MDM inhibitor, a PD-1 inhibitor, AKT inhibitor, or WEE1 inhibitor, can be an antibody and can be obtained commercially, isolated from an immunized animal or a monoclonal-producing hybridoma, or generated synthetically, such as by recombinant protein expression techniques.

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

Nucleic Acid Inhibitors

An anti-cancer immune therapeutic agent and/or a stimulator of p53 levels and/or p53 activity can be a nucleic acid directed towards the target to be inhibited, such as an antisense nucleic acid or an RNA interference nucleic acid directed towards the target to be inhibited, such as an siRNA directed towards the target to be inhibited.

An anti-cancer immune therapeutic agent, such as a PD-1 inhibitor, and/or a stimulator of p53 levels and/or p53 activity, such as an MDM2 and/or MDM4 inhibitor, AKT inhibitor, or WEE1 inhibitor can be a nucleic acid, such as an antisense nucleic acid or an RNA interference nucleic acid directed towards the target to be inhibited, such as an siRNA directed towards the target to be inhibited.

The term “siRNA” refers to a “small interfering RNA,” also known as a “short interfering RNA” which is a synthetic double stranded RNA which targets a specific mRNA for degradation, reducing or preventing translation of the mRNA in a cell. An siRNA is generally 15 to 40 base pairs in length, preferably 19 to 25 base pairs in length, and may be blunt ended or include a 3′ and/or 5′ overhang on each strand, wherein the overhang on each strand is independently 1, 2, 3, 4 or 5 nucleotides.

siRNA can be designed to specifically target an immune checkpoint protein, such as PD-1, PD-L1, or CTLA4.

siRNA effective to stimulate p53 levels and/or p53 activity can be designed, such as an siRNA directed to specifically target any one or more of: MDM2, MDM4, AKT, and WEE1.

An siRNA directed against a specified target can be obtained commercially or synthesized, see for example, Engelke, D. R., RNA Interference (RNAi): Nuts and Bolts of RNAi Technology, DNA Press LLC, Eagleville, Pa., 2003; and Herdewijn, P. (Ed.), Oligonucleotide Synthesis: Methods and Applications, Methods in Molecular Biology, Humana Press, 2004.

A CRISPR/Cas construct effective to stimulate p53 levels and/or p53 activity can be designed, such as a CRISPR/Cas construct directed to specifically target any one or more of: MDM2, MDM4, AKT, and WEE1.

A CRISPR/Cas construct can be designed as an anti-cancer immune therapeutic agent to specifically target an immune checkpoint protein, such as PD-1, PD-L1, or CTLA4.

CRISPR/Cas technologies are well-known in the art, such as described in CRISPR/Cas: A Laboratory Manual, Doudna and Mali (eds), Cold Spring Harbor Laboratory Press, Cold Spring Harbor, N.Y., USA, 2016, and can be used to generate a desired construct effective to stimulate p53 levels and/or p53 activity or as an anti-cancer immune therapeutic agent.

Other Inhibitors

GDC0068 has the structural formula:

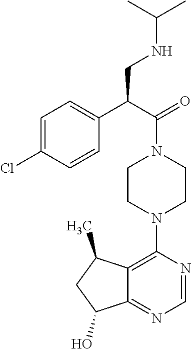 [SMILES: CC(C)NC[C@@H](C(=O)N1CCN(c2ncnc3c2[C@H](C)C[C@H]3O)CC1)c1ccc(Cl)cc1]

and is commercially available or can be synthesized according to standard methodology.

AZD5363 has the structural formula:

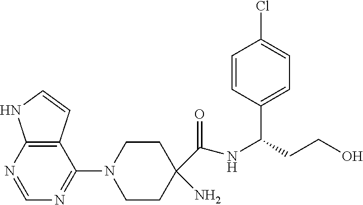 [SMILES: NC1(C(=O)N[C@@H](CCO)c2ccc(Cl)cc2)CCN(c2ncnc3[nH]ccc23)CC1]

and is commercially available or can be synthesized according to standard methodology.

MK1775 has the structural formula:

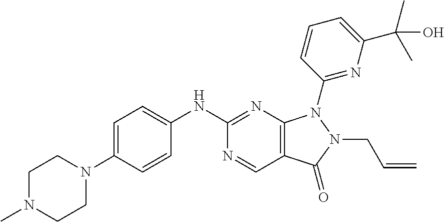 [SMILES: C=CCn1c(=O)c2cnc(Nc3ccc(N4CCN(C)CC4)cc3)nc2n1-c1cccc(C(C)(C)O)n1]

and is commercially available or can be synthesized according to standard methodology.

Compositions

Compositions are provided according to aspects of the present disclosure which include 1) an anti-cancer immune therapeutic agent, and 2) a stimulator of p53 levels and/or activity. According to aspects of the present disclosure, the anti-cancer immune therapeutic agent is a cell-based agent or a non-cell based agent.

According to aspects of the present disclosure, the anti-cancer immune therapeutic agent is a cell-based agent selected from: an NK cell-based anti-cancer immune therapeutic agent; and a CAR-T cell-based anti-cancer immune therapeutic agent.

According to aspects of the present disclosure, the anti-cancer immune therapeutic agent comprises is an immune checkpoint inhibitor.

According to aspects of the present disclosure, the anti-cancer immune therapeutic agent is an immune checkpoint inhibitor selected from the group consisting of: a PD-1 inhibitor, a PD-1 ligand (PD-L1) inhibitor, and a CTLA4 inhibitor.

According to aspects of the present disclosure, the anti-cancer immune therapeutic agent is an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4.

According to aspects of the present disclosure, the anti-cancer immune therapeutic agent is a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR).

According to aspects of the present disclosure, the stimulator of p53 levels and/or activity inhibits degradation of p53. According to aspects of the present disclosure, the anti-cancer immune therapeutic agent the stimulator of p53 levels and/or activity inhibits degradation of p53, such as an inhibitor of MDM2 and/or MDM4.

According to aspects of the present disclosure, the stimulator of p53 levels and/or activity is an inhibitor of AKT and/or an inhibitor of WEE1. According to aspects of the present disclosure, the stimulator of p53 levels and/or activity is a combination of an inhibitor of AKT and an inhibitor of WEE1. According to aspects of the present disclosure, the AKT inhibitor comprises is selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT. An included AKT inhibitor can be an AKT3 inhibitor. According to aspects of the present disclosure, the WEE1 inhibitor is a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Optionally, a composition according to aspects of the present disclosure includes a pharmaceutically acceptable carrier.

According to aspects of the present disclosure, an inventive composition excludes a CHK1 inhibitor and/or an mTOR inhibitor.

Compositions are provided according to aspects of the present disclosure which include an immune checkpoint inhibitor, an AKT inhibitor, and a WEE1 inhibitor, wherein the immune checkpoint inhibitor is selected from: a PD-1 inhibitor, a PD-L1 inhibitor, and a CTLA4 inhibitor. Optionally, the immune checkpoint inhibitor is an antibody. In a further option, the immune checkpoint inhibitor is an anti-PD-1 antibody, an anti-PD-L1 antibody, an anti-CTLA4 antibody, or an siRNA or a CRISPR/Cas knockdown construct directed to PD-1, PD-L1, or CTLA4. Optionally, a pharmaceutically acceptable carrier is included.

Compositions are provided according to aspects of the present disclosure which include: 1) an immune checkpoint inhibitor, 2) a WEE1 inhibitor, and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) an immune checkpoint inhibitor which is an anti-PD-1 antibody, an anti-PD-L1 antibody, an anti-CTLA4 antibody, or an siRNA or a CRISPR/Cas knockdown construct directed to PD-1, PD-L1, or CTLA4; 2) a WEE1 inhibitor; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) a PD-1 inhibitor selected from the group consisting of: nivolumab, pembrolizumab, and an siRNA or a CRISPR/Cas knockdown construct directed to PD-1; 2) a WEE1 inhibitor; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) a PD-L1 inhibitor selected from the group consisting of: atezolizumab, avelumab, and durvalumab, and an siRNA or a CRISPR/Cas knockdown construct directed to PD-L1; 2) a WEE1 inhibitor; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) a CTLA4 inhibitor: ipilimumab, and an siRNA or a CRISPR/Cas knockdown construct directed to CTLA4; 2) a WEE1 inhibitor; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) an immune checkpoint inhibitor, 2) a WEE1 inhibitor selected from the group consisting of: MK1775, a pharmaceutically acceptable salt, hydrate, amide or ester thereof, and an siRNA or a CRISPR/Cas knockdown construct directed to WEE1, and 3) an AKT inhibitor.

Compositions are provided according to aspects of the present disclosure which include: 1) an immune checkpoint inhibitor which is an anti-PD-1 antibody, an anti-PD-L1 antibody, an anti-CTLA4 antibody, or an siRNA or a CRISPR/Cas knockdown construct directed to PD-1, PD-L1, or CTLA4; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor. Optionally, a pharmaceutically acceptable carrier is included.

Compositions are provided according to aspects of the present disclosure which include: 1) a PD-1 inhibitor selected from the group consisting of: nivolumab, pembrolizumab, and an siRNA or a CRISPR/Cas knockdown construct directed to PD-1; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor.

Compositions are provided according to aspects of the present disclosure which include: 1) a PD-L1 inhibitor selected from the group consisting of: atezolizumab, avelumab, and durvalumab, and an siRNA or a CRISPR/Cas knockdown construct directed to PD-L1; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor. Optionally, a pharmaceutically acceptable carrier is included.

Compositions are provided according to aspects of the present disclosure which include: 1) a CTLA4 inhibitor: ipilimumab, and a siRNA or a CRISPR/Cas knockdown construct directed to CTLA4; 2) a WEE1 inhibitor selected from the group consisting of: MK1775, a pharmaceutically acceptable salt, hydrate, amide or ester thereof, and a siRNA or a CRISPR/Cas knockdown construct directed to WEE1; and 3) an AKT inhibitor. Optionally, a pharmaceutically acceptable carrier is included.

Compositions are provided according to aspects of the present disclosure which include: 1) an immune checkpoint inhibitor, 2) a WEE1 inhibitor selected from the group consisting of: MK1775, a pharmaceutically acceptable salt, hydrate, amide or ester thereof, and an siRNA or a CRISPR/Cas knockdown construct directed to WEE1, and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) an immune checkpoint inhibitor which is an anti-PD-1 antibody, an anti-PD-L1 antibody, an anti-CTLA4 antibody, or an siRNA or a CRISPR/Cas knockdown construct directed to PD-1, PD-L1, or CTLA4; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) a PD-1 inhibitor selected from the group consisting of: nivolumab, pembrolizumab, and an siRNA or a CRISPR/Cas knockdown construct directed to PD-1; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) a PD-L1 inhibitor selected from the group consisting of: atezolizumab, avelumab, and durvalumab, and an siRNA or a CRISPR/Cas knockdown construct directed to PD-L1; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

Compositions are provided according to aspects of the present disclosure which include: 1) a CTLA4 inhibitor: ipilimumab, and an siRNA or a CRISPR/Cas knockdown construct directed to CTLA4; 2) a WEE1 inhibitor selected from the group consisting of: MK1775, a pharmaceutically acceptable salt, hydrate, amide or ester thereof, and an siRNA or a CRISPR/Cas knockdown construct directed to WEE1; and 3) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT. Optionally, a pharmaceutically acceptable carrier is included.

A composition according to aspects of the present disclosure includes an immune checkpoint inhibitor, an AKT inhibitor, and a WEE1 inhibitor according to the disclosure generally includes about 0.1-99% of an immune checkpoint inhibitor, about 0.1-99% of an AKT inhibitor, about 0.1-99% of a WEE1 inhibitor; and optionally includes a pharmaceutically acceptable carrier.

A composition according to aspects of the present disclosure includes 1) one or more of: AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, according to the disclosure generally includes about 0.1-99% of component 1), about 0.1-99% of component 2) about 0.1-99% of component 3), and optionally a pharmaceutically acceptable carrier.

A composition according to aspects of the present disclosure includes 1) one or more of: AZD5363, GDC0068 and an siRNA or a CRISPR/Cas knockdown construct directed to AKT; 2) one or both of MK1775 and an siRNA or a CRISPR/Cas knockdown construct directed to WEE1; and 3) one or more immune checkpoint inhibitors selected from: an anti-PD-1 antibody, an anti-PD-L1 antibody, an anti-CTLA4 antibody, and an siRNA or a CRISPR/Cas knockdown construct directed to PD-1, PD-L1, or CTLA4; according to the disclosure generally includes about 0.1-99% of component 1), about 0.1-99% of component 2) about 0.1-99% of component 3), and optionally a pharmaceutically acceptable carrier.

A composition according to aspects of the present disclosure includes 1) one or more of: AZD5363, GDC0068 and an siRNA or a CRISPR/Cas knockdown construct directed to AKT; 2) one or both of MK1775 and an siRNA or a CRISPR/Cas knockdown construct directed to WEE1; and 3) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; according to the disclosure generally includes about 0.1-99% of component 1), about 0.1-99% of component 2) about 0.1-99% of component 3), and optionally a pharmaceutically acceptable carrier.

A pharmaceutical composition of the present disclosure may be in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. Liposomes and emulsions are well-known types of pharmaceutical formulations that can be used to deliver a pharmaceutical agent, particularly a hydrophobic pharmaceutical agent. Pharmaceutical compositions of the present disclosure generally include a pharmaceutically acceptable carrier such as an excipient, diluent and/or vehicle. Delayed release formulations of compositions and delayed release systems, such as semipermeable matrices of solid hydrophobic polymers can be used.

The term “pharmaceutically acceptable carrier” refers to a carrier which is suitable for use in a subject without undue toxicity or irritation to the subject and which is compatible with other ingredients included in a pharmaceutical composition.

Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alginates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present disclosure to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present disclosure may include a colorant, a stabilizer, a wetting agent, an emulsifying agent, a suspending agent, a sweetener, a flavoring, or a perfuming agent.

For example, a composition for parenteral administration may be formulated as an injectable liquid. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavoring, or a perfuming agent.

Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Ionotophoresis and/or sonophoresis can be used to enhance transdermal delivery.

Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount.

Ophthalmic formulations of one or more active agents can include ingredients such as a preservative, a buffer and a thickening agent.

Suitable surface-active agents useful as a pharmaceutically acceptable carrier or excipient in the pharmaceutical compositions of the present disclosure include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphthalene sulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphosphatidyl-choline and their mixtures.

Suitable non-ionic surfactants useful as pharmaceutically acceptable carriers or excipients in the pharmaceutical compositions of the present disclosure include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present disclosure include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual ” (MC Publishing Crop., Ridgewood, N.J., 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the disclosure. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Compositions and components of compositions, according to the present disclosure can be administered in formulations to increase efficacy and reduce toxicity, including use of nanoparticles, liposomes, controlled release agents, immediate release agents and polymeric conjugation approaches.

In particular aspects, a pharmaceutically acceptable carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate pharmaceutically acceptable carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

Compositions according to the present disclosure encompass stereoisomers of a WEE1 inhibitor. Compositions and pharmaceutical compositions according to the present disclosure encompass the individual enantiomers of a WEE1 inhibitor as well as wholly or partially racemic mixtures of any of these.

Compositions according to the present disclosure encompass stereoisomers of an AKT inhibitor. Compositions and pharmaceutical compositions according to the present disclosure encompass the individual enantiomers of an AKT inhibitor as well as wholly or partially racemic mixtures of any of these.

Compositions according to the present disclosure encompass stereoisomers of a PD-1 inhibitor. Compositions and pharmaceutical compositions according to the present disclosure encompass the individual enantiomers of an immune checkpoint inhibitor as well as wholly or partially racemic mixtures of any of these.

Compositions according to the present disclosure encompass stereoisomers of MK1775. Compositions and pharmaceutical compositions according to the present disclosure encompass the individual enantiomers of MK1775, as well as wholly or partially racemic mixtures of any of these.

Compositions according to the present disclosure encompass stereoisomers of GDC0068. Compositions according to the present disclosure encompass the individual enantiomers of GDC0068, as well as wholly or partially racemic mixtures of any of these.

Compositions according to the present disclosure encompass stereoisomers of AZD5363. Compositions and pharmaceutical compositions according to the present disclosure encompass the individual enantiomers of AZD5363, as well as wholly or partially racemic mixtures of any of these.

A derivative of an immune checkpoint inhibitor, an AKT inhibitor, or a WEE1 inhibitor is optionally included in a composition or method according to aspects of the present disclosure. The term “derivative” as used herein refers to a compound that is modified compared to a reference compound and which has similar or improved bioactivity compared to the reference compound.

According to aspects, an AKT inhibitor included in compositions and methods of the present disclosure specifically inhibits AKT, i.e. all of AKT1, AKT2 and AKT3 or one or more of AKT1, AKT2 and AKT3, but does not substantially inhibit non-AKT protein kinases.

According to aspects, a WEE1 inhibitor included in compositions and methods of the present disclosure specifically inhibits WEE1, i.e. both of WEE1A and WEE1B or one of WEE1A and WEE1B, but does not substantially inhibit non-WEE1 protein kinases.

The term “pharmaceutically acceptable salt” refers to salts which are suitable for use in a subject without undue toxicity or irritation to the subject and which are effective for their intended use.

Pharmaceutically acceptable salts include pharmaceutically acceptable acid addition salts and base addition salts. Pharmaceutically acceptable salts are well-known in the art, such as those detailed in S. M. Berge et al., J. Pharm. Sci., 66:1-19, 1977. Exemplary pharmaceutically acceptable salts are those suitable for use in a subject without undue toxicity or irritation to the subject and which are effective for their intended use which are formed with inorganic acids such as hydrochloric acid, hydrobromic acid, hydroiodic acid, nitric acid, phosphoric acid, sulfuric acid and sulfamic acid; organic acids such as acetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, formic acid, fumaric acid, glutamic acid, glycolic acid, glycerophosphoric acid, hemisulfic acid, heptanoic acid, hexanoic acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, trichloroacetic acid, trifluoroacetic acid and undecanoic acid; inorganic bases such as ammonia, hydroxide, carbonate, and bicarbonate of ammonium; organic bases such as primary, secondary, tertiary and quaternary amine compounds ammonium, arginine, betaine, choline, caffeine, diolamine, diethylamine, diethanolamine, 2-dimethylaminoethanol, 2-diethylaminoethanol, dicyclohexylamine, dicyclohexylamine, dibenzylamine, N, N-dibenzylphenethylamine, 1-ephenamine, N, N′-dibenzylethylenediamine, ethanolamine, ethylamine, ethylenediamine, glucosamine, histidine, hydrabamine, isopropylamine, 1h-imidazole, lysine, methylamine, N-ethylpiperidine, N-methylpiperidine, N-methylmorpholine, N, N-dimethylaniline, piperazine, trolamine, methylglucamine, purines, piperidine, pyridine, theobromine, tetramethylammonium compounds, tetraethylammonium compounds, trimethylamine, triethylamine, tripropylamine and tributylamine and metal cations such as aluminum, calcium, copper, iron, lithium, magnesium, manganese, potassium, sodium, and zinc.

Methods

Methods of treating cancer in a subject in need thereof are provided according to aspects of the present disclosure which include administering a combination of 1) an anti-cancer immune therapeutic agent, and 2) a stimulator of p53 levels and/or p53 activity, wherein the combination of 1) the anti-cancer immune therapeutic agent and 2) a stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more cell-based anti-cancer immune therapeutic agents selected from: an NK cell-based anti-cancer immune therapeutic agent; and a CAR-T cell-based anti-cancer immune therapeutic agent; and 2) a stimulator of p53 levels and/or p53 activity, wherein the combination of the one or more cell-based anti-cancer immune therapeutic agents, and the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more non-cell based anti-cancer immune therapeutic agents selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR); and 2) a stimulator of p53 levels and/or p53 activity, wherein the combination of the one or more non-cell based anti-cancer immune therapeutic agents and the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor and 2) a stimulator of p53 levels and/or p53 activity, wherein the combination of the immune checkpoint inhibitor, the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor, wherein the immune check point inhibitor is a PD-1 inhibitor, a PD-1 ligand (PD-L1) inhibitor, and/or a CTLA4 inhibitor and 2) a stimulator of p53 levels and/or p53 activity, wherein the combination of 1) the immune checkpoint inhibitor, and 2) a stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor wherein the immune checkpoint inhibitor is an anti-PD-1 antibody, an anti-PD-L1 antibody, an anti-CTLA4 antibody, or an siRNA or a CRISPR/Cas knockdown construct directed to PD-1, PD-L1, or CTLR4, and 2) a stimulator of p53 levels and/or p53 activity, wherein the combination of 1) the immune checkpoint inhibitor and 2) the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; and 2) a stimulator of p53 levels and/or p53 activity, wherein the combination of the immune checkpoint, inhibitor, and the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an anti-cancer immune therapeutic agent, 2) an AKT inhibitor, and 3) a WEE1 inhibitor, wherein the combination of 1) the anti-cancer immune therapeutic agent, 2) the AKT inhibitor and 3) the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more cell-based anti-cancer immune therapeutic agents selected from: an NK cell-based anti-cancer immune therapeutic agent; and a CAR-T cell-based anti-cancer immune therapeutic agent; 2) an AKT inhibitor; and 3) a WEE1 inhibitor, wherein the combination of the one or more cell-based anti-cancer immune therapeutic agents, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more non-cell based anti-cancer immune therapeutic agents selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR); 2) an AKT inhibitor; and 3) a WEE1 inhibitor, wherein the combination of the one or more non-cell based anti-cancer immune therapeutic agents, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor, 2) an AKT inhibitor, and 3) a WEE1 inhibitor, wherein the combination of the immune checkpoint inhibitor, the AKT inhibitor and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor, wherein the immune check point inhibitor is a PD-1 inhibitor, a PD-1 ligand (PD-L1) inhibitor, and/or a CTLA4 inhibitor, 2) an AKT inhibitor, and 3) a WEE1 inhibitor, wherein the combination of 1) the immune checkpoint inhibitor, the AKT inhibitor and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor wherein the immune checkpoint inhibitor is an anti-PD-1 antibody, an anti-PD-L1 antibody, an anti-CTLA4 antibody, or an siRNA or a CRISPR/Cas knockdown construct directed to PD-1, PD-L1, or CTLR4, 2) an AKT inhibitor, and 3) a WEE1 inhibitor, wherein the combination of 1) the immune checkpoint inhibitor, 2) the AKT inhibitor, and 3) the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor, wherein the combination of the immune checkpoint, inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an anti-cancer immune therapeutic agent, 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of 1) the anti-cancer immune therapeutic agent, 2) the AKT inhibitor and 3) the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more cell-based anti-cancer immune therapeutic agents selected from: an NK cell-based anti-cancer immune therapeutic agent; and a CAR-T cell-based anti-cancer immune therapeutic agent; 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT; and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of the one or more cell-based anti-cancer immune therapeutic agents, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more non-cell based anti-cancer immune therapeutic agents selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR); 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT; and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of the one or more non-cell based anti-cancer immune therapeutic agents, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present invention which include administering a combination of 1) an immune checkpoint inhibitor, 2) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT; and 3) a WEE1 inhibitor, wherein the combination of the immune checkpoint inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present invention which include administering a combination of 1) an immune checkpoint inhibitor, 2) an AKT inhibitor, and 3) a WEE1 inhibitor selected from the group consisting of: MK1775, a pharmaceutically acceptable salt, hydrate, amide or ester thereof and an siRNA or a CRISPR/Cas knockdown construct directed to WEE1, wherein the combination of the immune checkpoint inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor, 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of the immune checkpoint inhibitor, the AKT inhibitor and the WEE1 inhibitor is administered together in a single pharmaceutical formulation, in two pharmaceutical formulations, or in separate pharmaceutical formulations.

Methods of treating cancer in a subject in need thereof are provided according to aspects of the present invention which include administering a combination of: 1) an anti-PD-1 antibody, an anti-PD-L1 antibody, an anti-CTLA4 antibody, or an siRNA or a CRISPR/Cas knockdown construct directed to PD-1, PD-L1, or CTLA4; 2) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT; and 3) a WEE1 inhibitor, wherein the combination of the immune checkpoint inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present invention which include administering a combination of: 1) an immune checkpoint inhibitor wherein the immune checkpoint inhibitor is an anti-PD-1 antibody, an anti-PD-L1 antibody, an anti-CTLA4 antibody, or an siRNA or a CRISPR/Cas knockdown construct directed to PD-1, PD-L1, or CTLA4; 2) an AKT inhibitor; and 3) a WEE1 inhibitor selected from the group consisting of: MK1775, a pharmaceutically acceptable salt, hydrate, amide or ester thereof and an siRNA or a CRISPR/Cas knockdown construct directed to WEE1, wherein the combination of the immune checkpoint inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor, wherein the immune check point inhibitor is a PD-1 inhibitor, a PD-1 ligand (PD-L1) inhibitor, and/or a CTLA4 inhibitor, 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of 1) the immune checkpoint inhibitor, the AKT inhibitor and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor wherein the immune checkpoint inhibitor is an anti-PD-1 antibody, an anti-PD-L1 antibody, an anti-CTLA4 antibody, or an siRNA or a CRISPR/Cas knockdown construct directed to PD-1, PD-L1, or CTLR4, 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of 1) the immune checkpoint inhibitor, 2) the AKT inhibitor, and 3) the WEE1 inhibitor is administered together in a single pharmaceutical formulation, in two pharmaceutical formulations, or in separate pharmaceutical formulations.

Methods of treating cancer in a subject in need thereof are provided according to aspects of the present invention which include administering a combination of: 1) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor selected from the group consisting of: AZD5363, GDC0068, a combination of AZD5363 and GDC0068, a pharmaceutically acceptable salt, hydrate, amide or ester of any thereof, and an siRNA or a CRISPR/Cas knockdown construct directed to AKT; and 3) a WEE1 inhibitor, wherein the combination of the immune checkpoint inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present invention which include administering a combination of: 1) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor selected from the group consisting of: MK1775, a pharmaceutically acceptable salt, hydrate, amide or ester thereof and an siRNA or a CRISPR/Cas knockdown construct directed to WEE1, wherein the combination of the immune checkpoint inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT; and 3) a WEE1 inhibitor, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1, wherein the combination of the immune checkpoint, inhibitor, the AKT inhibitor, and the WEE1 inhibitor is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an anti-cancer immune therapeutic agent, and 2) a stimulator of p53 levels and/or p53 activity, wherein the stimulator of p53 levels and/or p53 activity inhibits degradation of p53, such as an inhibitor of MDM2 and/or MDM4, wherein the combination of 1) the anti-cancer immune therapeutic agent and 2) the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more cell-based anti-cancer immune therapeutic agents selected from: an NK cell-based anti-cancer immune therapeutic agent; and a CAR-T cell-based anti-cancer immune therapeutic agent; and 2) a stimulator of p53 levels and/or p53 activity, wherein the stimulator of p53 levels and/or p53 activity inhibits degradation of p53, such as an inhibitor of MDM2 and/or MDM4, wherein the combination of the one or more cell-based anti-cancer immune therapeutic agents, and the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more non-cell based anti-cancer immune therapeutic agents selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR); and 2) a stimulator of p53 levels and/or p53 activity, wherein the stimulator of p53 levels and/or p53 activity inhibits degradation of p53, such as an inhibitor of MDM2 and/or MDM4, wherein the combination of the one or more non-cell based anti-cancer immune therapeutic agents and the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor and 2) a stimulator of p53 levels and/or p53 activity, wherein the stimulator of p53 levels and/or p53 activity inhibits degradation of p53, such as an inhibitor of MDM2 and/or MDM4, wherein the combination of the immune checkpoint inhibitor, the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor, wherein the immune check point inhibitor is a PD-1 inhibitor, a PD-1 ligand (PD-L1) inhibitor, and/or a CTLA4 inhibitor and 2) a stimulator of p53 levels and/or p53 activity, wherein the stimulator of p53 levels and/or p53 activity inhibits degradation of p53, such as an inhibitor of MDM2 and/or MDM4, wherein the combination of 1) the immune checkpoint inhibitor, and 2) a stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of 1) an immune checkpoint inhibitor wherein the immune checkpoint inhibitor is an anti-PD-1 antibody, an anti-PD-L1 antibody, an anti-CTLA4 antibody, or an siRNA or a CRISPR/Cas knockdown construct directed to PD-1, PD-L1, or CTLR4, and 2) a stimulator of p53 levels and/or p53 activity, wherein the stimulator of p53 levels and/or p53 activity inhibits degradation of p53, such as an inhibitor of MDM2 and/or MDM4, wherein the combination of 1) the immune checkpoint inhibitor and 2) the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure which include administering a combination of: 1) one or more immune checkpoint inhibitors selected from: nivolumab, pembrolizumab, atezolizumab, avelumab, durvalumab, ipilimumab, and an siRNA or a CRISPR/Cas knockdown construct directed to PD-1, PD-L1, or CTLA4; and 2) a stimulator of p53 levels and/or p53 activity, wherein the stimulator of p53 levels and/or p53 activity inhibits degradation of p53, such as an inhibitor of MDM2 and/or MDM4, wherein the combination of the immune checkpoint, inhibitor, and the stimulator of p53 levels and/or p53 activity is administered together in a single pharmaceutical formulation, in two pharmaceutical formulations, or in separate pharmaceutical formulations.

Methods of treating cancer in a subject in need thereof are provided according to aspects of the present disclosure, wherein the cancer is melanoma, colorectal cancer, thyroid cancer, breast cancer, prostate cancer, sarcoma, glioblastoma, T-cell acute lymphoblastic leukaemia, lung cancer or liver cancer, and which include administering a combination of 1) an immune checkpoint inhibitor, 2) an AKT inhibitor, and 3) a WEE1 inhibitor, wherein the combination of the immune checkpoint inhibitor, AKT inhibitor and the WEE1 inhibitor is administered together as a combination formulation or separately.

Methods of treating cancer characterized by wild-type p53 in a subject in need thereof are provided according to aspects of the present disclosure, wherein the cancer is melanoma, colorectal cancer, thyroid cancer, breast cancer, prostate cancer, sarcoma, glioblastoma, T-cell acute lymphoblastic leukaemia, lung cancer or liver cancer, and which include administering a combination of 1) an immune checkpoint inhibitor, 2) an AKT inhibitor, and 3) a WEE1 inhibitor, wherein the combination of the immune checkpoint inhibitor, AKT inhibitor and the WEE1 inhibitor is administered together as a combination formulation or separately.

Methods of treating cancer in a subject in need thereof are provided according to aspects of the present disclosure which include administering a combination of 1) an immune checkpoint inhibitor, 2) an AKT inhibitor, and 3) a WEE1 inhibitor, wherein the immune checkpoint inhibitor, AKT inhibitor, and the WEE1 inhibitor are administered sequentially within a period of time selected from: one hour, two hours, four hours, eight hours, twelve hours, twenty-four hours, 2 days, 3 days, 4 days, 5 days, 6 days and 7 days.

Methods of treating cancer in a subject in need thereof are provided according to aspects of the present disclosure which include administering a combination of 1) an immune checkpoint inhibitor, 2) an AKT inhibitor, and 3) a WEE1 inhibitor, wherein the AKT inhibitor is an AKT3 inhibitor.

Cancers treated using methods and compositions described herein are characterized by abnormal cell proliferation including, but not limited to, pre-neoplastic hyperproliferation, cancer in-situ, neoplasms and metastasis, and include solid and non-solid tumors. Examples of cancers treated according to aspects of the present disclosure include, but are not limited to, lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer.

Cancers treated using methods and compositions according to aspects of the present disclosure are characterized by abnormal cell proliferation and AKT dysregulation.

Cancers treated using methods and compositions according to aspects of the present disclosure are characterized by high expression of AKT and/or WEE1 compared to normal cells. For example, prostate cancer treated using methods and compositions according to aspects of the present disclosure are characterized by high expression of AKT and/or WEE1 compared to normal prostate cells. Similarly, any cancer such as lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer characterized by high expression of AKT and/or WEE1 compared to normal cells of a corresponding normal tissue are treated using methods and compositions according to aspects of the present disclosure by a method including administering a combination of an AKT inhibitor and a WEE1 inhibitor.

Synergistic effects of combination compositions and treatments including administration of 1) an anti-cancer immune therapeutic agent, and 2) a stimulator of p53 levels and/or p53 activity are unexpectedly found as described herein.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, as a combination formulation or separately, wherein administration of the combination provides a synergistic effect.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, wherein the stimulator of p53 levels and/or p53 activity includes 1) an inhibitor of MDM2 and/or MDM4 and/or 2) an inhibitor of AKT and an inhibitor of WEE1, and wherein the anti-cancer immune therapeutic agent includes a cell-based agent or a non-cell based agent, as a combination formulation or separately, wherein administration of the combination provides a synergistic effect.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, wherein the stimulator of p53 levels and/or p53 activity includes 1) an inhibitor of MDM2 and/or MDM4 and/or 2) an inhibitor of AKT and an inhibitor of WEE1, and wherein the anti-cancer immune therapeutic agent includes a cell-based agent selected from: an NK cell-based anti-cancer immune therapeutic agent; and a CAR-T cell-based anti-cancer immune therapeutic agent, as a combination formulation or separately, wherein administration of the combination provides a synergistic effect.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, wherein the stimulator of p53 levels and/or p53 activity includes 1) an inhibitor of MDM2 and/or MDM4 and/or 2) an inhibitor of AKT and an inhibitor of WEE1, and wherein the anti-cancer immune therapeutic agent includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), as a combination formulation or separately, wherein administration of the combination provides a synergistic effect.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, wherein the stimulator of p53 levels and/or p53 activity includes 1) an inhibitor of MDM2 and/or MDM4 and/or 2) an inhibitor of AKT and an inhibitor of WEE1, and wherein the anti-cancer immune therapeutic agent includes an immune checkpoint inhibitor, as a combination formulation or separately, wherein administration of the combination provides a synergistic effect.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, wherein the stimulator of p53 levels and/or p53 activity includes 1) an inhibitor of MDM2 and/or MDM4 and/or 2) an inhibitor of AKT and an inhibitor of WEE1, and wherein the anti-cancer immune therapeutic agent includes an immune checkpoint inhibitor selected from the group consisting of: a PD-1 inhibitor, a PD-1 ligand (PD-L1) inhibitor, and a CTLA4 inhibitor, as a combination formulation or separately, wherein administration of the combination provides a synergistic effect.

Methods of treatment of a subject having, or at risk of having cancer characterized by wild-type p53 are provided according to aspects of the present disclosure which include administering an anti-cancer immune therapeutic agent, and a stimulator of p53 levels and/or p53 activity, wherein the stimulator of p53 levels and/or p53 activity includes 1) an inhibitor of MDM2 and/or MDM4 and/or 2) an inhibitor of AKT and an inhibitor of WEE1, and wherein the anti-cancer immune therapeutic agent includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, as a combination formulation or separately, wherein administration of the combination provides a synergistic effect.

Methods of treatment of a subject having, or at risk of having cancer characterized by wild-type p53 are provided according to aspects of the present disclosure which include administering a combination of: 1) one or more of AZD5363, GDC0068, and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, as a combination formulation of 1), 2), and 3); as a combination formulation of 1) and 2) with 3) separate, 2) and 3) with 1) separate, or 1: and 3) with 2) separate; or all of 1), 2), and 3) separately, wherein administration of the combination provides a synergistic effect.

Methods of treatment of a subject having, or at risk of having melanoma characterized by wild-type p53 are provided according to aspects of the present disclosure which include administering a combination of an immune checkpoint inhibitor, an AKT inhibitor, and a WEE1 inhibitor as a combination formulation or separately, wherein administration of the combination provides a synergistic effect.

Methods of treatment of a subject having, or at risk of having melanoma characterized by wild-type p53 are provided according to aspects of the present disclosure which include administering a combination of an immune checkpoint inhibitor, an AKT inhibitor, and a WEE1 inhibitor as a combination formulation or separately, wherein administration of the combination provides a synergistic effect.

Methods of treatment of a subject having, or at risk of having melanoma characterized by wild-type p53 are provided according to aspects of the present disclosure which include administering a combination of: 1) one or more of AZD5363, GDC0068, and an siRNA or a CRISPR/Cas knockdown construct directed to AKT; and 2) one or both of MK1775 and an siRNA or a CRISPR/Cas knockdown construct directed to WEE1; and 3) an immune checkpoint inhibitor, as a combination formulation of 1), 2), and 3); as a combination formulation of 1) and 2) with 3) separate, 2) and 3) with 1) separate, or 1: and 3) with 2) separate; or all of 1), 2), and 3) separately, wherein administration of the combination provides a synergistic effect.

Methods and compositions of the present disclosure can be used for prophylaxis as well as amelioration of signs and/or symptoms of cancer. The terms “treating” and “treatment” used to refer to treatment of a cancer in a subject include: preventing, inhibiting or ameliorating the cancer in the subject, such as slowing progression of the cancer and/or reducing or ameliorating a sign or symptom of the cancer.

A therapeutically effective amount of a combination of: 1) an anti-cancer immune therapeutic agent, and 2) a stimulator of p53 levels and/or p53 activity, administered as a combination formulation or separately, is an amount which has a beneficial effect in a subject having cancer or at risk for having cancer, such as a condition characterized by abnormal cell proliferation including, but not limited to, pre-neoplastic hyperproliferation, cancer in-situ, neoplasms, metastasis, a tumor, a benign growth or other condition responsive to a composition of the present disclosure, wherein a therapeutically effective amount of a composition of the present disclosure is effective to ameliorate or prevent one or more signs and/or symptoms of the condition.

A therapeutically effective amount of an immune checkpoint inhibitor, an AKT inhibitor and a WEE1 inhibitor, administered as a combination formulation or separately, is an amount which has a beneficial effect in a subject having cancer or at risk for having cancer, such as a condition characterized by abnormal cell proliferation including, but not limited to, pre-neoplastic hyperproliferation, cancer in-situ, neoplasms, metastasis, a tumor, a benign growth or other condition responsive to a composition of the present disclosure, wherein a therapeutically effective amount of a composition of the present disclosure is effective to ameliorate or prevent one or more signs and/or symptoms of the condition.

A subject treated according to methods and using compositions of the present disclosure can be mammalian or non-mammalian. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. A non-mammalian subject can be any non-mammal including, but not limited to, a bird such as a duck, goose, chicken, or turkey. Subjects can be either gender and can be any age. In aspects of methods of treatment according to the present disclosure, the subject is human. The terms “subject” and “patient” are used interchangeably herein.

According to aspects of the present disclosure, combination therapies include: (A) administration of a pharmaceutical combination composition including 1) an anti-cancer immune therapeutic agent, and 2) a stimulator of p53 levels and/or p53 activity, wherein the stimulator of p53 levels and/or p53 activity, formulated together in a single pharmaceutical composition; and/or (B) co-administration of 1) an anti-cancer immune therapeutic agent, and 2) a stimulator of p53 levels and/or p53 activity wherein the anti-cancer immune therapeutic agent, and the stimulator of p53 levels and/or p53 activity have not been formulated in the same composition. When using separate formulations, the anti-cancer immune therapeutic agent may be administered at the same time, intermittent times, staggered times, prior to, subsequent to, or combinations thereof, with reference to the stimulator of p53 levels and/or p53 activity.

According to aspects of the present disclosure, combination therapies include: (A) administration of a pharmaceutical combination composition including 1) an anti-cancer immune therapeutic agent; 2) an AKT inhibitor; and 3) a WEE1 inhibitor, formulated together in a single pharmaceutical composition; and/or (B) co-administration of: 1) an anti-cancer immune therapeutic agent; 2) an AKT inhibitor; and 3) a WEE1 inhibitor; wherein the anti-cancer immune therapeutic agent, AKT inhibitor, and the WEE1 inhibitor have not been formulated in the same composition or wherein only two of: the anti-cancer immune therapeutic agent, AKT inhibitor, and the WEE1 inhibitor are formulated in the same composition. When using separate formulations, the anti-cancer immune therapeutic agent, AKT inhibitor, and WEE1 inhibitor may be administered at the same time, intermittent times, staggered times, prior to, subsequent to, or combinations thereof, with reference to each of the other inhibitors or with reference to a combination of two of the other inhibitors.

According to aspects of the present disclosure, combination therapies include: (A) pharmaceutical compositions that include a pharmaceutical combination composition including: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, formulated together in a single pharmaceutical composition. According to aspects of the present disclosure, combination therapies include: (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, wherein the components 1), 2) and 3), have not been formulated in the same composition or wherein only two of the immune checkpoint inhibitor, AKT inhibitor, and the WEE1 inhibitor are formulated in the same composition. When using separate formulations, the immune checkpoint inhibitor, AKT inhibitor, and WEE1 inhibitor may be administered at the same time, intermittent times, staggered times, prior to, subsequent to, or combinations thereof, with reference to each of the other inhibitors or with reference to a combination of two of the other inhibitors.

According to aspects of the present disclosure, combination therapies include: (A) administration of a pharmaceutical combination composition including 1) an anti-cancer immune therapeutic agent selected from: an NK cell-based anti-cancer immune therapeutic agent; and a CAR-T cell-based anti-cancer immune therapeutic agent, and 2) a stimulator of p53 levels and/or p53 activity formulated together in a single pharmaceutical composition; and/or (B) co-administration of: 1) an anti-cancer immune therapeutic agent selected from: an NK cell-based anti-cancer immune therapeutic agent; and a CAR-T cell-based anti-cancer immune therapeutic agent, and 2) a stimulator of p53 levels and/or p53 activity wherein the anti-cancer immune therapeutic agent selected from: an NK cell-based anti-cancer immune therapeutic agent; and a CAR-T cell-based anti-cancer immune therapeutic agent, and the stimulator of p53 levels and/or p53 activity have not been formulated in the same composition. When using separate formulations, the anti-cancer immune therapeutic agent selected from: an NK cell-based anti-cancer immune therapeutic agent; and a CAR-T cell-based anti-cancer immune therapeutic agent may be administered at the same time, intermittent times, staggered times, prior to, subsequent to, or combinations thereof, with reference to the stimulator of p53 levels and/or p53 activity.

According to aspects of the present disclosure, combination therapies include: (A) administration of a pharmaceutical combination composition including 1) an immune checkpoint inhibitor, and 2) a stimulator of p53 levels and/or p53 activity formulated together in a single pharmaceutical composition; and/or (B) co-administration of: 1) an immune checkpoint inhibitor, and 2) a stimulator of p53 levels and/or p53 activity wherein the immune checkpoint inhibitor, and the stimulator of p53 levels and/or p53 activity have not been formulated in the same composition. When using separate formulations, the immune checkpoint inhibitor may be administered at the same time, intermittent times, staggered times, prior to, subsequent to, or combinations thereof, with reference to the stimulator of p53 levels and/or p53 activity.

According to aspects of the present disclosure, combination therapies include: (A) administration of a pharmaceutical combination composition including 1) an immune checkpoint inhibitor selected from: a PD-1 inhibitor, a PD-1 ligand (PD-L1) inhibitor, and a CTLA4 inhibitor, and 2) a stimulator of p53 levels and/or p53 activity formulated together in a single pharmaceutical composition; and/or (B) co-administration of: 1) an immune checkpoint inhibitor selected from: a PD-1 inhibitor, a PD-1 ligand (PD-L1) inhibitor, and a CTLA4 inhibitor, and 2) a stimulator of p53 levels and/or p53 activity wherein the immune checkpoint inhibitor selected from: a PD-1 inhibitor, a PD-1 ligand (PD-L1) inhibitor, and a CTLA4 inhibitor, and the stimulator of p53 levels and/or p53 activity have not been formulated in the same composition. When using separate formulations, the immune checkpoint inhibitor selected from: a PD-1 inhibitor, a PD-1 ligand (PD-L1) inhibitor, and a CTLA4 inhibitor, may be administered at the same time, intermittent times, staggered times, prior to, subsequent to, or combinations thereof, with reference to the stimulator of p53 levels and/or p53 activity.

According to aspects of the present disclosure, combination therapies include: (A) administration of a pharmaceutical combination composition including 1) an immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and 2) a stimulator of p53 levels and/or p53 activity formulated together in a single pharmaceutical composition; and/or (B) co-administration of: 1) an immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and 2) a stimulator of p53 levels and/or p53 activity wherein the immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and the stimulator of p53 levels and/or p53 activity have not been formulated in the same composition. When using separate formulations, the immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, may be administered at the same time, intermittent times, staggered times, prior to, subsequent to, or combinations thereof, with reference to the stimulator of p53 levels and/or p53 activity.

According to aspects of the present disclosure, combination therapies include: (A) administration of a pharmaceutical combination composition including 1) an anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), and 2) a stimulator of p53 levels and/or p53 activity formulated together in a single pharmaceutical composition; and/or (B) co-administration of: 1) an anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), and 2) a stimulator of p53 levels and/or p53 activity wherein the anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), and the stimulator of p53 levels and/or p53 activity have not been formulated in the same composition. When using separate formulations, the anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), may be administered at the same time, intermittent times, staggered times, prior to, subsequent to, or combinations thereof, with reference to the stimulator of p53 levels and/or p53 activity.

According to aspects of the present disclosure, combination therapies include: (A) administration of a pharmaceutical combination composition including an immune checkpoint inhibitor, an AKT inhibitor, and a WEE1 inhibitor, formulated together with one or more additional therapeutic agents in a single pharmaceutical composition; (B) co-administration of an immune checkpoint inhibitor, an AKT inhibitor, a WEE1 inhibitor, and optionally one or more additional pharmaceutical agents, wherein the immune checkpoint inhibitor, AKT inhibitor, WEE1 inhibitor, and the optional one or more additional pharmaceutical agents, have not been formulated in the same composition; and/or (C) co-administration of an immune checkpoint inhibitor, an AKT inhibitor, a WEE1 inhibitor, and optionally one or more additional pharmaceutical agents, wherein two, three, or more, but not all, of: the immune checkpoint inhibitor, AKT inhibitor, the WEE1 inhibitor, and the optional one or more additional pharmaceutical agents, are formulated in the same composition. When using separate formulations, each of the immune checkpoint inhibitor, AKT inhibitor, the WEE1 inhibitor, and optionally one or more additional pharmaceutical agents, may be administered at the same time, intermittent times, staggered times, prior to, subsequent to, or combinations thereof, with reference to each of the other components.

According to aspects of the present disclosure, combination therapies include: (A) administration of a pharmaceutical combination composition including 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; an AKT inhibitor; and 3) a WEE1 inhibitor, formulated together in a single pharmaceutical composition; and/or (B) co-administration of: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor; wherein the immune checkpoint inhibitor, AKT inhibitor, and the WEE1 inhibitor have not been formulated in the same composition or wherein only two of: the immune checkpoint inhibitor, AKT inhibitor, and the WEE1 inhibitor are formulated in the same composition. When using separate formulations, the immune checkpoint inhibitor, AKT inhibitor, and WEE1 inhibitor may be administered at the same time, intermittent times, staggered times, prior to, subsequent to, or combinations thereof, with reference to each of the other inhibitors or with reference to a combination of two of the other inhibitors.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, formulated together in a single pharmaceutical composition. According to aspects of the present disclosure, combination therapies include: (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, wherein the components 1), 2) and 3), have not been formulated in the same composition or wherein only two of the immune checkpoint inhibitor, AKT inhibitor, and the WEE1 inhibitor are formulated in the same composition. When using separate formulations, the immune checkpoint inhibitor, AKT inhibitor, and WEE1 inhibitor may be administered at the same time, intermittent times, staggered times, prior to, subsequent to, or combinations thereof, with reference to each of the other inhibitors or with reference to a combination of two of the other inhibitors.

According to aspects of the present disclosure, combination therapies include: (A) administration of a pharmaceutical combination composition including 1) an immune checkpoint inhibitor selected from an anti-PD-1 antibody, an anti-PD-L1 antibody, an anti-CTLA4 antibody, and an siRNA or a CRISPR/Cas knockdown construct directed to PD-1, PD-L1, or CTLA4; 2) an AKT inhibitor; and 3) a WEE1 inhibitor; formulated together with 4) one or more additional therapeutic agents in a single pharmaceutical composition; (B) co-administration of: 1) an immune checkpoint inhibitor selected from an anti-PD-1 antibody, an anti-PD-L1 antibody, an anti-CTLA4 antibody, and an siRNA or a CRISPR/Cas knockdown construct directed to PD-1, PD-L1, or CTLA4; 2) an AKT inhibitor; 3) a WEE1 inhibitor; and 4) one or more additional pharmaceutical agents, wherein the immune checkpoint inhibitor, AKT inhibitor, WEE1 inhibitor, and the one or more additional pharmaceutical agents have not been formulated in the same composition; and/or (C) co-administration of 1) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4; 2) an AKT inhibitor; 3) a WEE1 inhibitor; and 4) one or more additional pharmaceutical agents; wherein two, three, or more, but not all, of the immune checkpoint inhibitor, AKT inhibitor, the WEE1 inhibitor, and the one or more additional pharmaceutical agents, are formulated in the same composition. When using separate formulations, each of the immune checkpoint inhibitor, AKT inhibitor, the WEE1 inhibitor, and one or more additional pharmaceutical agents may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor; formulated together in a single pharmaceutical composition; (B) co-administration of: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor, wherein the immune checkpoint inhibitor, AKT inhibitor, and WEE1 inhibitor, have not been formulated in the same composition; and/or (C) co-administration of: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor; wherein two, three, or more, but not all, of: the immune checkpoint inhibitor, the AKT inhibitor, and the WEE1 inhibitor, are formulated in the same composition. When using separate formulations, each of the immune checkpoint inhibitor, the AKT inhibitor, and the WEE1 inhibitor, may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, in a single pharmaceutical composition; (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, wherein the one or more of AZD5363, GDC0068 and an siRNA or a CRISPR/Cas knockdown construct directed to AKT, the one or both of MK1775 and an siRNA or a CRISPR/Cas knockdown construct directed to WEE1, and the immune checkpoint inhibitor, have not been formulated in the same composition; and/or (C) co-administration of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor, wherein two, three, or more, but not all, of: the one or more of AZD5363, GDC0068 and an siRNA or a CRISPR/Cas knockdown construct directed to AKT; one or both of MK1775 and an siRNA or a CRISPR/Cas knockdown construct directed to WEE1; and the immune checkpoint inhibitor, are formulated in the same composition. When using separate formulations, the one or more of AZD5363, GDC0068 and an siRNA or a CRISPR/Cas knockdown construct directed to AKT; one or both of MK1775 and an siRNA or a CRISPR/Cas knockdown construct directed to WEE1; and the immune checkpoint inhibitor, may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4; formulated together in a single pharmaceutical composition; (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4, wherein the one or more of AZD5363, GDC0068 and an siRNA or a CRISPR/Cas knockdown construct directed to AKT, the one or both of MK1775 and an siRNA or a CRISPR/Cas knockdown construct directed to WEE1, and the immune checkpoint inhibitor; and/or (C) co-administration of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4, wherein two, three, or more, but not all, of: the one or more of AZD5363, GDC0068 and an siRNA or a CRISPR/Cas knockdown construct directed to AKT; one or both of MK1775 and an siRNA or a CRISPR/Cas knockdown construct directed to WEE1; and the immune checkpoint inhibitor, are formulated in the same composition. When using separate formulations, the one or more of AZD5363, GDC0068 and an siRNA or a CRISPR/Cas knockdown construct directed to AKT; one or both of MK1775 and an siRNA or a CRISPR/Cas knockdown construct directed to WEE1; and the immune checkpoint inhibitor, may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; and 2) one or both of MK1775 and an siRNA or a CRISPR/Cas knockdown construct directed to WEE1; and 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4, wherein the one or more of AZD5363, GDC0068 and an siRNA or a CRISPR/Cas knockdown construct directed to AKT, the one or both of MK1775 and an siRNA or a CRISPR/Cas knockdown construct directed to WEE1, and the immune checkpoint inhibitor, have not been formulated in the same composition; and/or (C) co-administration of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; and 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4, wherein two, three, or more, but not all, of: the one or more of AZD5363, GDC0068 and an siRNA or a CRISPR/Cas knockdown construct directed to AKT; one or both of MK1775 and an siRNA or a CRISPR/Cas knockdown construct directed to WEE1; andthe immune checkpoint inhibitor, are formulated in the same composition. When using separate formulations, the one or more of AZD5363, GDC0068 and an siRNA or a CRISPR/Cas knockdown construct directed to AKT; one or both of MK1775 and an siRNA or a CRISPR/Cas knockdown construct directed to WEE1; and the immune checkpoint inhibitor, may be administered at the same time, intermittent times, staggered times, prior to, subsequent to, or combinations thereof, with reference to each of the other components.

According to aspects of the present disclosure, combination therapies include: (A) administration of a pharmaceutical combination composition including 1) an anti-cancer immune therapeutic agent, and 2) a stimulator of p53 levels and/or p53 activity, wherein the stimulator of p53 levels and/or p53 activity inhibits degradation of p53, such as an inhibitor of MDM2 and/or MDM4, formulated together in a single pharmaceutical composition; and/or (B) co-administration of 1) an anti-cancer immune therapeutic agent, and 2) stimulator of p53 levels and/or p53 activity, wherein the stimulator of p53 levels and/or p53 activity inhibits degradation of p53, such as an inhibitor of MDM2 and/or MDM4, wherein the anti-cancer immune therapeutic agent, and the stimulator of p53 levels and/or p53 activity have not been formulated in the same composition. When using separate formulations, the anti-cancer immune therapeutic agent may be administered at the same time, intermittent times, staggered times, prior to, subsequent to, or combinations thereof, with reference to the stimulator of p53 levels and/or p53 activity, wherein the stimulator of p53 levels and/or p53 activity inhibits degradation of p53, such as an inhibitor of MDM2 and/or MDM4.

According to aspects of the present disclosure, combination therapies include: (A) administration of a pharmaceutical combination composition including 1) an anti-cancer immune therapeutic agent selected from: an NK cell-based anti-cancer immune therapeutic agent; and a CAR-T cell-based anti-cancer immune therapeutic agent, and 2) a stimulator of p53 levels and/or p53 activity, wherein the stimulator of p53 levels and/or p53 activity inhibits degradation of p53, such as an inhibitor of MDM2 and/or MDM4, formulated together in a single pharmaceutical composition; and/or (B) co-administration of: 1) an anti-cancer immune therapeutic agent selected from: an NK cell-based anti-cancer immune therapeutic agent; and a CAR-T cell-based anti-cancer immune therapeutic agent, and 2) a stimulator of p53 levels and/or p53 activity, wherein the stimulator of p53 levels and/or p53 activity inhibits degradation of p53, such as an inhibitor of MDM2 and/or MDM4, wherein the anti-cancer immune therapeutic agent selected from: an NK cell-based anti-cancer immune therapeutic agent; and a CAR-T cell-based anti-cancer immune therapeutic agent, and the stimulator of p53 levels and/or p53 activity, wherein the stimulator of p53 levels and/or p53 activity inhibits degradation of p53, such as an inhibitor of MDM2 and/or MDM4, have not been formulated in the same composition. When using separate formulations, the anti-cancer immune therapeutic agent selected from: an NK cell-based anti-cancer immune therapeutic agent; and a CAR-T cell-based anti-cancer immune therapeutic agent may be administered at the same time, intermittent times, staggered times, prior to, subsequent to, or combinations thereof, with reference to the stimulator of p53 levels and/or p53 activity, wherein the stimulator of p53 levels and/or p53 activity inhibits degradation of p53, such as an inhibitor of MDM2 and/or MDM4.

According to aspects of the present disclosure, combination therapies include: (A) administration of a pharmaceutical combination composition including 1) an immune checkpoint inhibitor, and 2) a stimulator of p53 levels and/or p53 activity, wherein the stimulator of p53 levels and/or p53 activity inhibits degradation of p53, such as an inhibitor of MDM2 and/or MDM4, formulated together in a single pharmaceutical composition; and/or (B) co-administration of: 1) an immune checkpoint inhibitor, and 2) a stimulator of p53 levels and/or p53 activity, wherein the stimulator of p53 levels and/or p53 activity inhibits degradation of p53, such as an inhibitor of MDM2 and/or MDM4, wherein the immune checkpoint inhibitor, and the stimulator of p53 levels and/or p53 activity have not been formulated in the same composition. When using separate formulations, the immune checkpoint inhibitor may be administered at the same time, intermittent times, staggered times, prior to, subsequent to, or combinations thereof, with reference to the stimulator of p53 levels and/or p53 activity, wherein the stimulator of p53 levels and/or p53 activity inhibits degradation of p53, such as an inhibitor of MDM2 and/or MDM4.

According to aspects of the present disclosure, combination therapies include: (A) administration of a pharmaceutical combination composition including 1) an immune checkpoint inhibitor selected from: a PD-1 inhibitor, a PD-1 ligand (PD-L1) inhibitor, and a CTLA4 inhibitor, and 2) a stimulator of p53 levels and/or p53 activity, wherein the stimulator of p53 levels and/or p53 activity inhibits degradation of p53, such as an inhibitor of MDM2 and/or MDM4, formulated together in a single pharmaceutical composition; and/or (B) co-administration of: 1) an immune checkpoint inhibitor selected from: a PD-1 inhibitor, a PD-1 ligand (PD-L1) inhibitor, and a CTLA4 inhibitor, and 2) a stimulator of p53 levels and/or p53 activity, wherein the stimulator of p53 levels and/or p53 activity inhibits degradation of p53, such as an inhibitor of MDM2 and/or MDM4, wherein the immune checkpoint inhibitor selected from: a PD-1 inhibitor, a PD-1 ligand (PD-L1) inhibitor, and a CTLA4 inhibitor, and the stimulator of p53 levels and/or p53 activity have not been formulated in the same composition. When using separate formulations, the immune checkpoint inhibitor selected from: a PD-1 inhibitor, a PD-1 ligand (PD-L1) inhibitor, and a CTLA4 inhibitor, may be administered at the same time, intermittent times, staggered times, prior to, subsequent to, or combinations thereof, with reference to the stimulator of p53 levels and/or p53 activity, wherein the stimulator of p53 levels and/or p53 activity inhibits degradation of p53, such as an inhibitor of MDM2 and/or MDM4.

According to aspects of the present disclosure, combination therapies include: (A) administration of a pharmaceutical combination composition including 1) an immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and 2) a stimulator of p53 levels and/or p53 activity, wherein the stimulator of p53 levels and/or p53 activity inhibits degradation of p53, such as an inhibitor of MDM2 and/or MDM4, formulated together in a single pharmaceutical composition; and/or (B) co-administration of: 1) an immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and 2) a stimulator of p53 levels and/or p53 activity, wherein the stimulator of p53 levels and/or p53 activity inhibits degradation of p53, such as an inhibitor of MDM2 and/or MDM4, wherein the immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and the stimulator of p53 levels and/or p53 activity have not been formulated in the same composition. When using separate formulations, the immune checkpoint inhibitor selected from: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, may be administered at the same time, intermittent times, staggered times, prior to, subsequent to, or combinations thereof, with reference to the stimulator of p53 levels and/or p53 activity, wherein the stimulator of p53 levels and/or p53 activity inhibits degradation of p53, such as an inhibitor of MDM2 and/or MDM4.

According to aspects of the present disclosure, combination therapies include: (A) administration of a pharmaceutical combination composition including 1) an anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), and 2) a stimulator of p53 levels and/or p53 activity, wherein the stimulator of p53 levels and/or p53 activity inhibits degradation of p53, such as an inhibitor of MDM2 and/or MDM4, formulated together in a single pharmaceutical composition; and/or (B) co-administration of: 1) an anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), and 2) a stimulator of p53 levels and/or p53 activity, wherein the stimulator of p53 levels and/or p53 activity inhibits degradation of p53, such as an inhibitor of MDM2 and/or MDM4, and wherein the anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), and the stimulator of p53 levels and/or p53 activity have not been formulated in the same composition. When using separate formulations, the anti-cancer immune therapeutic agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR), may be administered at the same time, intermittent times, staggered times, prior to, subsequent to, or combinations thereof, with reference to the stimulator of p53 levels and/or p53 activity, wherein the stimulator of p53 levels and/or p53 activity inhibits degradation of p53, such as an inhibitor of MDM2 and/or MDM4.

Optionally, an additional therapeutic agent may be administered, wherein the additional therapeutic agent is formulated with an anti-cancer immune therapeutic agent and/or with a stimulator of p53 levels and/or p53 activity.

Combinations of an immune checkpoint inhibitor, an AKT inhibitor, a WEE1 inhibitor, and one or more additional therapeutic agents are administered according to aspects of the present disclosure.

Combinations of: 1) an immune checkpoint inhibitor selected from: a PD-1 inhibitor, a PD-L1 inhibitor, and a CTLA4 inhibitor; 2) an AKT inhibitor, 3) a WEE1 inhibitor; and 4) one or more additional therapeutic agents, are administered according to aspects of the present disclosure.

Combinations of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: an anti-PD-1 antibody, an anti-PD-L1 antibody, an anti-CTLA4 antibody, or an siRNA or a CRISPR/Cas knockdown construct directed to PD-1, PD-L1, or CTLA4; and 4) one or more additional therapeutic agents, are administered according to aspects of the present disclosure.

Combinations of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; and 4) one or more additional therapeutic agents, are administered according to aspects of the present disclosure.

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present disclosure include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal anti-inflammatory agents, steroids and vasoactive agents.

According to aspects of the present disclosure, combination therapies include: (A) administration of a pharmaceutical combination composition including: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; and 3) a WEE1 inhibitor; formulated together with 4) one or more additional therapeutic agents in a single pharmaceutical composition; (B) co-administration of: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; 3) a WEE1 inhibitor; and 4) one or more additional pharmaceutical agents; wherein the immune checkpoint inhibitor, AKT inhibitor, WEE1 inhibitor, and the one or more additional pharmaceutical agents have not been formulated in the same composition; and/or (C) co-administration of: 1) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; 2) an AKT inhibitor; 3) a WEE1 inhibitor; and 4) one or more additional pharmaceutical agents; wherein two, three, or more, but not all, of: the immune checkpoint inhibitor, AKT inhibitor, the WEE1 inhibitor, and the one or more additional pharmaceutical agents are formulated in the same composition. When using separate formulations, each of the immune checkpoint inhibitor, AKT inhibitor, the WEE1 inhibitor, and one or more additional pharmaceutical agents may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor; and 4) one or more additional therapeutic agents, in a single pharmaceutical composition; (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor; and 4) one or more additional pharmaceutical agents, wherein the one or more of AZD5363, GDC0068 and an siRNA or a CRISPR/Cas knockdown construct directed to AKT, the one or both of MK1775 and an siRNA or a CRISPR/Cas knockdown construct directed to WEE1, the immune checkpoint inhibitor, and the one or more additional pharmaceutical agents have not been formulated in the same composition; and/or (C) co-administration of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor; and 4) one or more additional pharmaceutical agents, wherein two, three, or more, but not all, of: the one or more of AZD5363, GDC0068 and an siRNA or a CRISPR/Cas knockdown construct directed to AKT; one or both of MK1775 and an siRNA or a CRISPR/Cas knockdown construct directed to WEE1; the immune checkpoint inhibitor; and the one or more additional pharmaceutical agents are formulated in the same composition. When using separate formulations, the one or more of AZD5363, GDC0068 and an siRNA or a CRISPR/Cas knockdown construct directed to AKT; one or both of MK1775 and an siRNA or a CRISPR/Cas knockdown construct directed to WEE1; the immune checkpoint inhibitor; and the one or more additional pharmaceutical agents may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4; formulated together with 4) one or more additional therapeutic agents, in a single pharmaceutical composition; (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4; and 4) one or more additional pharmaceutical agents, wherein the one or more of AZD5363, GDC0068 and an siRNA or a CRISPR/Cas knockdown construct directed to AKT, the one or both of MK1775 and an siRNA or a CRISPR/Cas knockdown construct directed to WEE1, the immune checkpoint inhibitor, and the one or more additional pharmaceutical agents have not been formulated in the same composition; and/or (C) co-administration of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: an anti-PD-1 antibody, an anti-PD-L1 antibody, an anti-CTLA4 antibody, and an siRNA or a CRISPR/Cas knockdown construct directed to PD-1, PD-L1, or CTLA4; and 4) one or more additional pharmaceutical agents, wherein two, three, or more, but not all, of: the one or more of AZD5363, GDC0068 and an siRNA or a CRISPR/Cas knockdown construct directed to AKT; one or both of MK1775 and an siRNA or a CRISPR/Cas knockdown construct directed to WEE1; the immune checkpoint inhibitor; and the one or more additional pharmaceutical agents are formulated in the same composition. When using separate formulations, the one or more of AZD5363, GDC0068 and an siRNA or a CRISPR/Cas knockdown construct directed to AKT; one or both of MK1775 and an siRNA or a CRISPR/Cas knockdown construct directed to WEE1; the immune checkpoint inhibitor; and the one or more additional pharmaceutical agents may be administered at the same time, intermittent times, staggered times, prior to, subsequent to, or combinations thereof, with reference to each of the other components.

According to aspects of the present disclosure, combination therapies include: (A) administration of a pharmaceutical combination composition including: 1) one or more of AZD5363, GDC0068 and an siRNA or a CRISPR/Cas knockdown construct directed to AKT; and 2) one or both of MK1775 and an siRNA or a CRISPR/Cas knockdown construct directed to WEE1; 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; and 4) one or more additional therapeutic agents, in a single pharmaceutical composition; (B) co-administration of 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; and 4) one or more additional pharmaceutical agents, wherein the one or more of AZD5363, GDC0068 and an siRNA or a CRISPR/Cas knockdown construct directed to AKT, the one or both of MK1775 and an siRNA or a CRISPR/Cas knockdown construct directed to WEE1, the immune checkpoint inhibitor, and the one or more additional pharmaceutical agents have not been formulated in the same composition; and/or (C) co-administration of: 1) one or more of AZD5363, GDC0068 and an siRNA or a CRISPR/Cas knockdown construct directed to AKT; 2) one or both of MK1775 and an siRNA or a CRISPR/Cas knockdown construct directed to WEE1; 3) an immune checkpoint inhibitor selected from: nivolumab, pembrolizumab, atezolizumab, avelumab, durvalumab, ipilimumab, and an siRNA or a CRISPR/Cas knockdown construct directed to PD-1, PD-L1, or CTLA4; and 4) one or more additional pharmaceutical agents, wherein two, three, or more, but not all, of: the one or more of AZD5363, GDC0068 and an siRNA or a CRISPR/Cas knockdown construct directed to AKT; one or both of MK1775 and an siRNA or a CRISPR/Cas knockdown construct directed to WEE1; the immune checkpoint inhibitor; and the one or more additional pharmaceutical agents are formulated in the same composition. When using separate formulations, the one or more of AZD5363, GDC0068 and an siRNA or a CRISPR/Cas knockdown construct directed to AKT; one or both of MK1775 and an siRNA or a CRISPR/Cas knockdown construct directed to WEE1; the immune checkpoint inhibitor; and the one or more additional pharmaceutical agents may be administered at the same time, intermittent times, staggered times, prior to, subsequent to, or combinations thereof, with reference to each of the other components.

According to aspects of the present disclosure, the immune checkpoint inhibitor, AKT inhibitor, and the WEE1 inhibitor are administered together daily, administered together twice daily or administered together more often in one day.

According to aspects of the present disclosure, the immune checkpoint inhibitor, the AKT inhibitor, and the WEE1 inhibitor are all administered separately daily, all administered separately twice daily or all administered separately more often in one day.

According to aspects of the present disclosure, the immune checkpoint inhibitor, the AKT inhibitor, and the WEE1 inhibitor are administered sequentially within a period of time selected from: one hour, two hours, four hours, eight hours, twelve hours, twenty-four hours, 2 days, 3 days, 4 days, 5 days, 6 days or 1 week in a method of treatment of cancer in a subject.

According to aspects of the present disclosure, the AKT inhibitor is administered weekly, twice weekly, three times in a week, every other day, daily, administered twice daily or administered more often in one day, the WEE1 inhibitor is administered less often or more often than the AKT inhibitor, and the immune checkpoint inhibitor is administered less often or more often than the AKT inhibitor, in a treatment for cancer to achieve a synergistic effect of the combined administration to the subject.

According to aspects of the present disclosure, the immune checkpoint inhibitor, the AKT inhibitor, and the WEE1 inhibitor are administered together or separately at the same time, intermittent times, or staggered times during a treatment period which can be from 1 day to 100 days, such as 1 day to 2 days, 2 day to 3 days, 3 day to 5 days, 5 day to 7 days, 7 days to 14 days, 14 days to 21 days, 21 days to 28 days, 28 days to 35 days, 35 days to 50 days or 50 days to 60 days and which may include one or more periods in which the amount of the immune checkpoint inhibitor, and/or AKT inhibitor, and/or WEE1 inhibitor is increased or decreased, in which the identity of the particular immune checkpoint inhibitor, and/or AKT inhibitor, and/or the WEE1 inhibitor is changed, or in which no treatment is given.

Combination treatments can allow for reduced effective dosage and increased therapeutic index of the combination treatment of: 1) an anti-cancer immune therapeutic agent, and 2) a stimulator of p53 levels and/or p53 activity, as a combination formulation or separately.

Additional Pharmaceutical Agents

An additional pharmaceutical agent is an anti-cancer agent according to aspects of the present disclosure.

Anti-cancer agents are described, for example, in Goodman et al., Goodman and Gilman's The Pharmacological Basis of Therapeutics, 8th Ed., Macmillan Publishing Co., 1990.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bevacizumab, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, cobimetinib, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, vemurafenib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

According to aspects of the present disclosure, one or more correlative biomarkers of therapeutic activity of an immune checkpoint inhibitor, an AKT inhibitor, and a WEE1 inhibitor administered as a combination treatment of the present disclosure to treat cancer in a subject in need thereof are assayed to assess treatment of the cancer in the subject.

Biomarkers of apoptosis are correlative biomarkers of therapeutic activity of a combination treatment of the present disclosure to treat cancer in a subject in need thereof and an increase in one or more biomarkers of apoptosis in cancer cells is indicative of efficacy of the treatment of cancer in a subject in need thereof. Biomarkers of apoptosis include, but are not limited to, detection of DNA fragmentation, characteristic morphological changes distinct from necrosis and activation of caspase-3. Biomarkers of apoptosis are measured according to standard methodologies, for example as described herein.

According to aspects of the present disclosure, assays for effects of combination treatment of the present disclosure are used to monitor a subject. Thus, for example, a test sample is obtained from the subject before treatment according to a method of the present disclosure and at one or more times during and/or following treatment in order to assess effectiveness of the treatment. In a further example, a test sample is obtained from the subject at various times in order to assess the course or progress of disease or healing.

In particular aspects, one or more additional biomarkers are assayed in a test sample obtained from a subject to aid in monitoring treatment according to aspects of a method of treatment of the present disclosure.

Apoptosis of cancer cells is assayed in a test sample obtained from a subject to aid in monitoring treatment with a pharmaceutical composition of the present disclosure.

Expression and/or activity of p53 is assayed in a test sample obtained from the subject to aid in monitoring treatment according to aspects of a method of treatment of the present disclosure.

Expression and/or activity of MDM2 and/or MDM4 is assayed in a test sample obtained from the subject to aid in monitoring treatment according to aspects of a method of treatment of the present disclosure.

Expression and/or activity of an immune checkpoint, and/or AKT and/or WEE1 is assayed in a test sample obtained from the subject to aid in monitoring treatment according to a method of treatment of the present disclosure.

Optionally, a method of treating cancer in a subject in need thereof further includes an adjunct anti-cancer treatment. An adjunct anti-cancer treatment can be a radiation treatment of a subject or an affected area of a subject's body.

The dosage of an 1) an anti-cancer immune therapeutic agent, and 2) a stimulator of p53 levels and/or p53 activity, and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

The dosage of an immune checkpoint inhibitor, an AKT inhibitor, a WEE1 inhibitor, and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

In particular aspects of inventive methods, the amount of the anti-cancer immune therapeutic agent, and the stimulator of p53 levels and/or p53 activity administered in a combination treatment is less than an amount of the anti-cancer immune therapeutic agent, and the stimulator of p53 levels and/or p53 activity necessary to achieve a therapeutic effect if the subject is treated with any of these agents alone. Thus, in particular aspects of the present disclosure, the amount of the anti-cancer immune therapeutic agent, and the stimulator of p53 levels and/or p53 activity, administered in combination, in a treatment of cancer in a subject is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90% or more, less than an amount of the anti-cancer immune therapeutic agent, and the stimulator of p53 levels and/or p53 activity, necessary to achieve a therapeutic effect when administered without a combination treatment of the present disclosure.

In particular aspects of inventive methods, the amount of the anti-cancer immune therapeutic agent, and/or AKT inhibitor, and/or WEE1 inhibitor administered in a combination treatment is less than an amount of the anti-cancer immune therapeutic agent, and/or AKT inhibitor, and/or WEE1 inhibitor necessary to achieve a therapeutic effect if the subject is treated with any of these agents alone. Thus, in particular aspects of the present disclosure, the amount of the anti-cancer immune therapeutic agent, and/or an AKT inhibitor, and/or a WEE1 inhibitor, administered in combination, in a treatment of cancer in a subject is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90% or more, less than an amount of the anti-cancer immune therapeutic agent, and/or AKT inhibitor, and/or WEE1 inhibitor, necessary to achieve a therapeutic effect when administered without a combination treatment of the present disclosure.

In particular aspects of inventive methods, the amount of the immune checkpoint inhibitor, and/or AKT inhibitor, and/or WEE1 inhibitor administered in a combination treatment is less than an amount of the immune checkpoint inhibitor, and/or

AKT inhibitor, and/or WEE1 inhibitor necessary to achieve a therapeutic effect if the subject is treated with any of these agents alone. Thus, in particular aspects of the present disclosure, the amount of an immune checkpoint inhibitor, and/or an AKT inhibitor, and/or a WEE1 inhibitor, administered in combination, in a treatment of cancer in a subject is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90% or more, less than an amount of the immune checkpoint inhibitor, and/or AKT inhibitor, and/or WEE1 inhibitor, necessary to achieve a therapeutic effect when administered without a combination treatment of the present disclosure.

In general it is contemplated that a daily dosage of an anti-cancer immune therapeutic agent, a stimulator of p53 levels and/or p53 activity, and any optional additional therapeutic agent is in the range of about 0.001 to 100 milligrams per kilogram of a subject's body weight. A daily dose may be administered as two or more divided doses to obtain the desired effect. A pharmaceutical composition including any one or more of: an anti-cancer immune therapeutic agent, a stimulator of p53 levels and/or p53 activity, and any optional additional therapeutic agent, may also be formulated for sustained release to obtain desired results.

In general it is contemplated that a daily dosage of an immune checkpoint inhibitor, an AKT inhibitor, a WEE1 inhibitor, and any optional additional therapeutic agent is in the range of about 0.001 to 100 milligrams per kilogram of a subject's body weight. A daily dose may be administered as two or more divided doses to obtain the desired effect. A pharmaceutical composition including any one or more of: an immune checkpoint inhibitor, an AKT inhibitor, a WEE1 inhibitor, and any optional additional therapeutic agent, may also be formulated for sustained release to obtain desired results.

In particular aspects of inventive methods, an AKT inhibitor is administered in doses of 0.1 mg/day to 1 g/day, such as 0.1 mg/day to 0.25 mg/day, 0.25 mg/day to 0.5 mg/day, 0.5 mg/day to 0.75 mg/day, 0.75 mg/day to 1 mg/day, 0.25 mg/day to 500 mg/day, 0.5 mg/day to 200 mg/day, 0.75 mg/day to 100 mg/day, 1 mg/day to 2 mg/day, such as 2 mg/day to 5 mg/day, 5 mg/day to 10 mg/day, 5 mg/day to 20 mg/day, 10 mg/day to 20 mg/day, 20 mg/day to 30 mg/day, 30 mg/day to 40 mg/day, 40 mg/day to 50 mg/day, 40 mg/day to 60 mg/day, 60 mg/day to 70 mg/day, 70 mg/day to 80 mg/day, 80 mg/day to 90 mg/day, 90 mg/day to 95 mg/day, 95 mg/day to 100 mg/day, 100 mg/day to 150 mg/day, 150 mg/day to 200 mg/day, 200 mg/day to 250 mg/day, 250 mg/day to 300 mg/day, 300 mg/day to 350 mg/day, 350 mg/day to 400 mg/day, 400 mg/day to 450 mg/day, 450 mg/day to 500 mg/day, 500 mg/day to 550 mg/day, 550 mg/day to 600 mg/day, 600 mg/day to 650 mg/day, 650 mg/day to 700 mg/day, 700 mg/day to 750 mg/day, 750 mg/day to 800 mg/day, 800 mg/day to 850 mg/day, 850 mg/day to 900 mg/day, 900 mg/day to 950 mg/day or 950 mg/day to 1 g/day in a combination treatment with a WEE1 inhibitor and an immune checkpoint inhibitor.

In particular aspects of inventive methods, a WEE1 inhibitor is administered in doses of 0.1 mg/day to 1 g/day, such as 0.1 mg/day to 0.25 mg/day, 0.25 mg/day to 0.5 mg/day, 0.5 mg/day to 0.75 mg/day, 0.75 mg/day to 1 mg/day, 0.25 mg/day to 500 mg/day, 0.5 mg/day to 200 mg/day, 0.75 mg/day to 100 mg/day, 1 mg/day to 2 mg/day, such as 2 mg/day to 5 mg/day, 5 mg/day to 10 mg/day, 5 mg/day to 20 mg/day, 10 mg/day to 20 mg/day, 20 mg/day to 30 mg/day, 30 mg/day to 40 mg/day, 40 mg/day to 50 mg/day, 40 mg/day to 60 mg/day, 60 mg/day to 70 mg/day, 70 mg/day to 80 mg/day, 80 mg/day to 90 mg/day, 90 mg/day to 95 mg/day, 95 mg/day to 100 mg/day, 100 mg/day to 150 mg/day, 150 mg/day to 200 mg/day, 200 mg/day to 250 mg/day, 250 mg/day to 300 mg/day, 300 mg/day to 350 mg/day, 350 mg/day to 400 mg/day, 400 mg/day to 450 mg/day, 450 mg/day to 500 mg/day, 500 mg/day to 550 mg/day, 550 mg/day to 600 mg/day, 600 mg/day to 650 mg/day, 650 mg/day to 700 mg/day, 700 mg/day to 750 mg/day, 750 mg/day to 800 mg/day, 800 mg/day to 850 mg/day, 850 mg/day to 900 mg/day, 900 mg/day to 950 mg/day or 950 mg/day to 1 g/day in a combination treatment with an AKT inhibitor and an immune checkpoint inhibitor.

In particular aspects of inventive methods, an anti-cancer immune therapeutic agent, such as an immune checkpoint inhibitor, is administered in doses of 0.1 mg/day to 1 g/day, such as 0.1 mg/day to 0.25 mg/day, 0.25 mg/day to 0.5 mg/day, 0.5 mg/day to 0.75 mg/day, 0.75 mg/day to 1 mg/day, 0.25 mg/day to 500 mg/day, 0.5 mg/day to 200 mg/day, 0.75 mg/day to 100 mg/day, 1 mg/day to 2 mg/day, such as 2 mg/day to 5 mg/day, 5 mg/day to 10 mg/day, 5 mg/day to 20 mg/day, 10 mg/day to 20 mg/day, 20 mg/day to 30 mg/day, 30 mg/day to 40 mg/day, 40 mg/day to 50 mg/day, 40 mg/day to 60 mg/day, 60 mg/day to 70 mg/day, 70 mg/day to 80 mg/day, 80 mg/day to 90 mg/day, 90 mg/day to 95 mg/day, 95 mg/day to 100 mg/day, 100 mg/day to 150 mg/day, 150 mg/day to 200 mg/day, 200 mg/day to 250 mg/day, 250 mg/day to 300 mg/day, 300 mg/day to 350 mg/day, 350 mg/day to 400 mg/day, 400 mg/day to 450 mg/day, 450 mg/day to 500 mg/day, 500 mg/day to 550 mg/day, 550 mg/day to 600 mg/day, 600 mg/day to 650 mg/day, 650 mg/day to 700 mg/day, 700 mg/day to 750 mg/day, 750 mg/day to 800 mg/day, 800 mg/day to 850 mg/day, 850 mg/day to 900 mg/day, 900 mg/day to 950 mg/day or 950 mg/day to 1 g/day in a combination treatment with a stimulator of p53 levels and/or p53 activity, such as an AKT inhibitor and a WEE1 inhibitor.

In particular aspects of inventive methods, an AKT inhibitor and a WEE1 inhibitor are administered in a ratio (mole:mole) in the range of 0.1:100 to 100:0.1, such as 0.25:50, 0.5:25, 0.75:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:0.75, 25:0.5 or 50:0.25. According to further particular aspects of inventive methods, an AKT inhibitor and a WEE1 inhibitor are administered in a ratio (mole:mole) in the range of 1:1.25, 0.15:1, 0.31:1, 0.63:1, 1.25:1, 12:1, 128:1, 16:1, 2.5:1, 32:1, 64:1 or 1:2.5.

In particular aspects of inventive methods, an anti-cancer immune therapeutic agent, such as an immune checkpoint inhibitor, and a WEE1 inhibitor are administered in a ratio (mole:mole) in the range of 0.1:100 to 100:0.1, such as 0.25:50, 0.5:25, 0.75:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:0.75, 25:0.5 or 50:0.25. According to further particular aspects of inventive methods, an anti-cancer immune therapeutic agent, such as an immune checkpoint inhibitor, and a WEE1 inhibitor are administered in a ratio (mole:mole) in the range of 1:1.25, 0.15:1, 0.31:1, 0.63:1, 1.25:1, 12:1, 128:1, 16:1, 2.5:1, 32:1, 64:1 or 1:2.5.

In particular aspects of inventive methods, an anti-cancer immune therapeutic agent, such as an immune checkpoint inhibitor, and an AKT inhibitor are administered in a ratio (mole:mole) in the range of 0.1:100 to 100:0.1, such as 0.25:50, 0.5:25, 0.75:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:0.75, 25:0.5 or 50:0.25. According to further particular aspects of inventive methods, an anti-cancer immune therapeutic agent, such as an immune checkpoint inhibitor, and an AKT inhibitor are administered in a ratio (mole:mole) in the range of 1:1.25, 0.15:1, 0.31:1, 0.63:1, 1.25:1, 12:1, 128:1, 16:1, 2.5:1, 32:1, 64:1 or 1:2.5.

In particular aspects of inventive methods, the amount of the adjunct anti-cancer treatment and/or anti-cancer agent administered is less than an amount of the adjunct anti-cancer treatment and/or anti-cancer agent necessary to achieve a therapeutic effect if administered without a combination treatment of the present disclosure including administration of an anti-cancer immune therapeutic agent, such as an immune checkpoint inhibitor, and a stimulator of p53 levels and/or p53 activity, such as an AKT inhibitor and a WEE1 inhibitor. Thus, in particular aspects of the present disclosure, the amount of an anti-cancer treatment and/or agent administered is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the adjunct anti-cancer treatment and/or agent necessary to achieve a therapeutic effect when administered without a combination treatment of the present disclosure including administration of a combination of an anti-cancer immune therapeutic agent, and a stimulator of p53 levels and/or p53 activity such as a combination of an immune checkpoint inhibitor, an AKT inhibitor and a WEE1 inhibitor.

Methods of the present disclosure include administration of a pharmaceutical composition of the present disclosure by a route of administration including, but not limited to, oral, rectal, nasal, pulmonary, epidural, ocular, otic, intraarterial, intracardiac, intracerebroventricular, intradermal, intravenous, intramuscular, intraperitoneal, intraosseous, intrathecal, intravesical, subcutaneous, topical, transdermal, and transmucosal, such as by sublingual, buccal, vaginal, and inhalational, routes of administration.

Methods of treating cancer are provided according to aspects of the present disclosure which include obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for one or more markers of apoptosis, thereby monitoring effectiveness of administering the combination.

Methods of treating cancer are provided according to aspects of the present disclosure which include obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for activity of a mitogen-activated protein kinase-signaling pathway, thereby monitoring effectiveness of administering the combination.

Methods of treating cancer are provided according to aspects of the present disclosure which include obtaining a first sample containing or suspected of containing cancer cells from the subject prior to administering the combination of the combination of the anti-cancer immune therapeutic agent and the stimulator of p53 levels and/or p53 activity; obtaining a second sample containing or suspected of containing cancer cells from the subject after administering the combination; and assaying the first and second samples for AKT dysregulation, thereby monitoring effectiveness of administering the combination.

Methods of treating cancer are provided according to aspects of the present disclosure which include obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for p53 expression and/or an associated gene selected from the group consisting of: CLCA2, PVRL4, SULF2, CDKN1a, BTG2, ACTA2, TP5363, FDXR, GDF15, IGFBP5 and ADAM19, wherein an increase in p53 expression and/or expression of an associated gene selected from the group consisting of: CLCA2, PVRL4, SULF2, CDKN1a, BTG2, ACTA2, TP5363, FDXR, GDF15, IGFBP5 and ADAM19, is an indicator of an anti-cancer cell effect of treatment with the combination, thereby monitoring effectiveness of administering the combination.

Methods of treating cancer are provided according to aspects of the present disclosure which include obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for FOXM1 expression and/or expression of an associated gene selected from the group consisting of: TMPO, ANP32E, SMC4, KIF20B, ASPM, DEPDC1, NCAPG, CENPE, wherein a decrease in expression of FOXM1 and/or an associated gene selected from the group consisting of: TMPO, ANP32E, SMC4, KIF20B, ASPM, DEPDC1, NCAPG and CENPE, is an indicator of an anti-cancer cell effect of treatment with the combination, thereby monitoring effectiveness of administering the combination.

Methods of treating cancer are provided according to aspects of the present disclosure which include obtaining a first sample containing or suspected of containing cancer cells from the subject prior to administering a combination of an immune checkpoint inhibitor, an AKT inhibitor, and a WEE1 inhibitor; obtaining a second sample containing or suspected of containing cancer cells from the subject after administering the combination of the immune checkpoint inhibitor, the AKT inhibitor, and the WEE1 inhibitor; and assaying the first and second samples for one or more markers of apoptosis and/or for activity of a mitogen-activated protein kinase-signaling pathway, thereby monitoring effectiveness of administering the combination of the immune checkpoint inhibitor, the AKT inhibitor, and the WEE1 inhibitor.

Methods of treating cancer are provided according to aspects of the present disclosure which include obtaining a first sample containing or suspected of containing cancer cells from the subject prior to administering a combination of an immune checkpoint inhibitor, an AKT inhibitor, and a WEE1 inhibitor; obtaining a second sample containing or suspected of containing cancer cells from the subject after administering the combination of the immune checkpoint inhibitor, the AKT inhibitor, and the WEE1 inhibitor; and assaying the first and second samples for one or more markers of AKT dysregulation, thereby monitoring effectiveness of administering the combination of the AKT inhibitor and the WEE1 inhibitor.

Methods of treating cancer are provided according to aspects of the present disclosure which include obtaining a first sample containing or suspected of containing cancer cells from the subject prior to administering a combination of an immune checkpoint inhibitor, an AKT inhibitor, and a WEE1 inhibitor; obtaining a second sample containing or suspected of containing cancer cells from the subject after administering the combination of the immune checkpoint inhibitor, the AKT inhibitor, and the WEE1 inhibitor; and assaying the first and second samples for p53 expression and/or expression of a downstream factor in the p53 transcriptional pathway selected from the group consisting of: chloride channel accessory 2 (CLCA2), nectin cell adhesion molecule 4 (PVRL4), sulfatase 2 (SULF2), cyclin dependent kinase inhibitor 1A (CDKN1a), BTG anti-proliferation factor 2 (BTG2), actin, alpha 2, smooth muscle, aorta (ACTA2), tumor protein p53 (TP53), ferredoxin reductase (FDXR), growth differentiation factor 15 (GDF15), insulin like growth factor binding protein 5 (IGFBP5) and ADAM metallopeptidase domain 19 (ADAM19), wherein an increase in p53 expression, and/or a downstream factor selected from the group consisting of: CLCA2, PVRL4, SULF2, CDKN1a, BTG2, ACTA2, TP53, FDXR, GDF15, IGFBP5 and ADAM19, is an indicator of an anti-cancer cell effect of treatment with the combination of the immune checkpoint inhibitor, the AKT inhibitor, and the WEE1 inhibitor, thereby monitoring effectiveness of administering the combination of the immune checkpoint inhibitor, the AKT inhibitor, and the WEE1 inhibitor.

Methods of treating cancer are provided according to aspects of the present disclosure which include obtaining a first sample containing or suspected of containing cancer cells from the subject prior to administering the combination of an immune checkpoint inhibitor, an AKT inhibitor, and a WEE1 inhibitor; obtaining a second sample containing or suspected of containing cancer cells from the subject after administering the combination of the immune checkpoint inhibitor, the AKT inhibitor, and the WEE1 inhibitor; and assaying the first and second samples for forkhead box M1 (FOXM1) expression and/or expression of an associated downstream protein in this transcriptional pathway selected from the group consisting of: thymopoietin (TMPO), acidic nuclear phosphoprotein 32 family member E (ANP32E), structural maintenance of chromosomes 4 (SMC4), kinesin family member 20B (KIF20B), abnormal spindle microtubule assembly (ASPM), DEP domain containing 1 (DEPDC1), non-SMC condensin I complex subunit G (NCAPG) and centromere protein E (CENPE), wherein a decrease in FOXM1 expression and/or an associated downstream protein selected from the group consisting of: TMPO, ANP32E, SMC4, KIF20B, ASPM, DEPDC1, NCAPG and CENPE, is an indicator of an anti-cancer cell effect of treatment with the combination of the immune checkpoint inhibitor, the AKT inhibitor, and the WEE1 inhibitor, thereby monitoring effectiveness of administering the combination of the immune checkpoint inhibitor, the AKT inhibitor, and the WEE1 inhibitor.

Assays for expression of one or more markers of apoptosis, activity of a mitogen-activated protein kinase-signaling pathway, AKT dysregulation, p53 expression and/or expression of a downstream factor in the p53 transcriptional pathway selected from the group consisting of: CLCA2, PVRL4, SULF2, CDKN1a, BTG2, ACTA2, TP53, FDXR, GDF15, IGFBP5 and ADAM19; and/or FOXM1 expression and/or expression of an associated downstream protein in the FOXM1 transcriptional pathway selected from the group consisting of: TMPO, ANP32E, SMC4, KIF20B, ASPM, DEPDC1, NCAPG and CENPE, are performed using standard techniques such as nucleic acid assays, spectrometric assays, immunoassays and functional assays. One or more standards and/or controls can be used to allow quantitative determination of target marker in a sample.

Standards and controls suitable for assays are well-known in the art and the standard and/or control used can be any appropriate standard and/or control.

Methods of treatment of a subject having, or at risk of having cancer characterized by constitutive activation of MAP (mitogen-activated protein) kinase-signaling pathway through BRAF, KIT and/or RAS mutations are provided according to aspects of the present disclosure which include administering a combination of: 1) an anti-cancer immune therapeutic agent, and 2) a stimulator of p53 levels and/or p53 activity, as a combination formulation or separately, wherein administration of the combination provides a synergistic effect.

Methods of treatment of a subject having, or at risk of having cancer characterized by constitutive activation of MAP (mitogen-activated protein) kinase-signaling pathway through BRAF, KIT and/or RAS mutations are provided according to aspects of the present disclosure which include administering a combination of an immune checkpoint inhibitor, an AKT inhibitor, and a WEE1 inhibitor as a combination formulation or separately, wherein administration of the combination provides a synergistic effect.

^{V600E}BRAF is the most frequent genetic alteration occurring in 50% of sporadic melanoma; see Sullivan R J et al., Journal of Skin Cancer 2011; 2011:423239. Mutations in BRAF are also common in many other cancers and ^{V600E}BRAF is commonly found in colorectal and thyroid cancers. Mutations in KIT and N-RAS also activate MAPK signaling in 2 to 6% and 15 to 20% of cutaneous melanomas respectively, which like BRAF mutation regulates diverse cellular processes including proliferation, survival and metastases, see Sullivan R J et al., Journal of Skin Cancer 2011, 2011:423239; and Flaherty K T et al., The New England Journal of Medicine, 2010, 363(9):809-19.

The mutation status of BRAF, KIT, RAS and/or p53 can be assayed in a test sample obtained from a subject.

The mutation status of BRAF, KIT, RAS and/or p53 can be assayed by any of various methodologies including, but not limited to, protein or peptide sequencing, nucleic acid assay and immunoassay.

Assays for detecting BRAF, KIT, RAS and/or p53 nucleic acids, particularly mRNA or cDNA, include, but are not limited to, sequencing; polymerase chain reactions (PCR) such as RT-PCR; dot blot; in situ hybridization; Northern blot; and RNase protection.

A test sample can be any biological fluid, cell or tissue of a subject that includes or is suspected of including cancer cells or circulating DNA derived from cancer cells, illustratively including blood, plasma, serum, urine, saliva, ascites, cerebrospinal fluid, cerebroventricular fluid, pleural fluids, pulmonary and bronchial lavage samples, mucous, sweat, tears, semen, bladder wash samples, amniotic fluid, lymph, peritoneal fluid, synovial fluid, bone marrow aspirate, tumor cells or tissue, organ cells or tissue, such as biopsy material.

Immunoassay methods can be used to assay BRAF, KIT, RAS and/or p53 mutation status in a sample, including, but not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay.

Commercial Packages

Commercial packages are provided according to aspects of the present disclosure which include 1) an anti-cancer immune therapeutic agent, and 2) a stimulator of p53 levels and/or p53 activity.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises a cell-based agent or a non-cell based agent.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises a cell-based agent selected from: an NK cell-based anti-cancer immune therapeutic agent; and a CAR-T cell-based anti-cancer immune therapeutic agent.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR).

Commercial packages are provided according to aspects of the present disclosure which include 1) an anti-cancer immune therapeutic agent, and 2) a stimulator of p53 levels and/or p53 activity, wherein the stimulator of p53 levels and/or p53 activity inhibits degradation of p53.

Commercial packages are provided according to aspects of the present disclosure which include 1) an anti-cancer immune therapeutic agent, and 2) a stimulator of p53 levels and/or p53 activity, wherein the stimulator of p53 levels and/or p53 activity comprises an inhibitor of MDM2 and/or MDM4.

Commercial packages are provided according to aspects of the present disclosure which include 1) an anti-cancer immune therapeutic agent, and 2) a stimulator of p53 levels and/or p53 activity, wherein the stimulator of p53 levels and/or p53 activity comprises an inhibitor of AKT and/or an inhibitor of WEE1.

Commercial packages are provided according to aspects of the present disclosure which include 1) an anti-cancer immune therapeutic agent, and 2) a stimulator of p53 levels and/or p53 activity, wherein the stimulator of p53 levels and/or p53 activity comprises an inhibitor of AKT and/or an inhibitor of WEE1, and wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT.

Commercial packages are provided according to aspects of the present disclosure which include 1) an anti-cancer immune therapeutic agent, and 2) a stimulator of p53 levels and/or p53 activity, wherein the stimulator of p53 levels and/or p53 activity comprises an inhibitor of AKT and/or an inhibitor of WEE1, and wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: a PD-1 inhibitor, a PD-1 ligand (PD-L1) inhibitor, and a CTLA4 inhibitor.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: a PD-1 inhibitor, a PD-1 ligand (PD-L1) inhibitor, and a CTLA4 inhibitor, and wherein the stimulator of p53 levels and/or p53 activity comprises an inhibitor of AKT and/or an inhibitor of WEE1, and wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, and wherein the stimulator of p53 levels and/or p53 activity comprises an inhibitor of AKT and/or an inhibitor of WEE1, and wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: a PD-1 inhibitor, a PD-1 ligand (PD-L1) inhibitor, and a CTLA4 inhibitor, wherein the stimulator of p53 levels and/or p53 activity comprises an inhibitor of AKT and/or an inhibitor of WEE1, and wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, wherein the stimulator of p53 levels and/or p53 activity comprises an inhibitor of AKT and/or an inhibitor of WEE1, and wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: a PD-1 inhibitor, a PD-1 ligand (PD-L1) inhibitor, and a CTLA4 inhibitor, and wherein the stimulator of p53 levels and/or p53 activity comprises an inhibitor of AKT and/or an inhibitor of WEE1, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT, and wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Commercial packages are provided according to aspects of the present disclosure which include 1) an anti-cancer immune therapeutic agent, and 2) a stimulator of p53 levels and/or p53 activity, wherein the anti-cancer immune therapeutic agent comprises an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4, wherein the stimulator of p53 levels and/or p53 activity comprises an inhibitor of AKT and/or an inhibitor of WEE1, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT and wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Commercial packages are provided according to aspects of the present disclosure which include 1) an anti-cancer immune therapeutic agent, and 2) a stimulator of p53 levels and/or p53 activity, wherein the stimulator of p53 levels and/or p53 activity comprises an inhibitor of AKT and/or an inhibitor of WEE1, and wherein the AKT inhibitor comprises an AKT3 inhibitor.

Commercial packages are provided according to aspects of the present disclosure which include 1) an anti-cancer immune therapeutic agent, and 2) a stimulator of p53 levels and/or p53 activity, wherein the commercial package excludes a CHK1 inhibitor and/or an mTOR inhibitor.

Instructions for administering the immune checkpoint inhibitor, AKT inhibitor, and the WEE1 inhibitor, in combination or separately, are included according to aspects of the disclosure.

One or more ancillary components is optionally included in commercial packages of the present disclosure, such as a buffer or diluent.

Embodiments of inventive compositions and methods are illustrated in the following examples. These examples are provided for illustrative purposes and are not considered limitations on the scope of inventive compositions and methods.

Examples

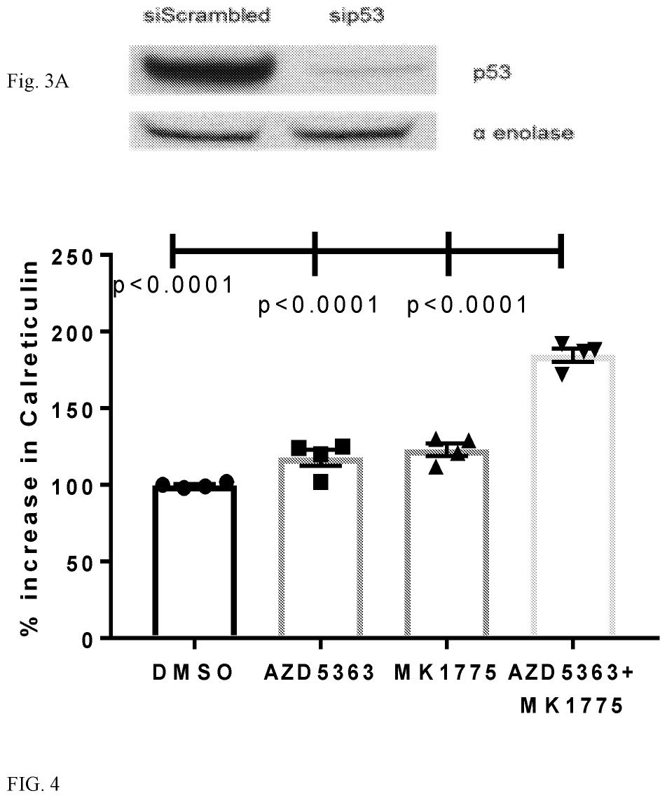

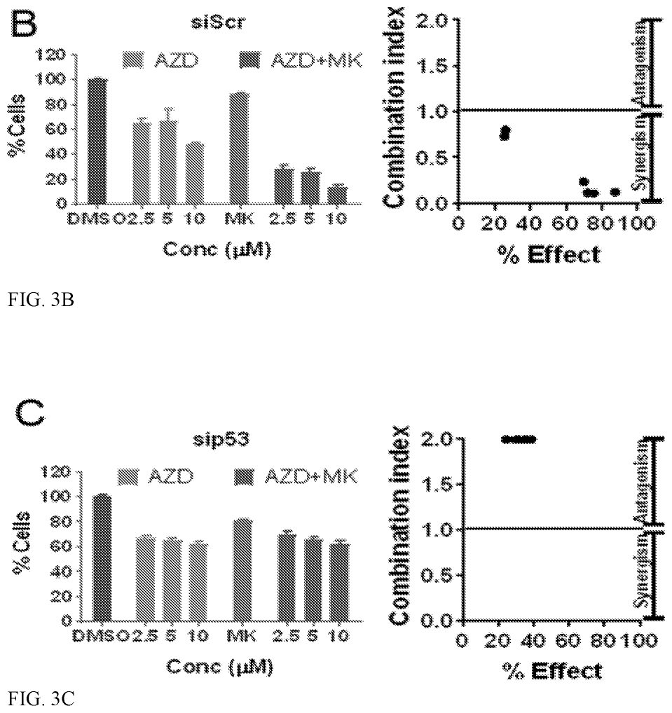

Wildtype p53 activity critically regulates the efficacy of AKT/WEE1 targeting. Specifically, wildtype p53 protein levels were knocked down in UACC 903 cells and then treated with pharmacological agents MK1775 and AZD5363. Knockdown of p53 protein levels is shown in FIG. 3A and effect of the drug combination on synergistic cell growth inhibition using scrambled siRNA is shown in FIG. 3B. CI value of <0.8 indicated that combined inhibition of AKT and WEE1 was highly synergistic when wildtype p53 protein was present in the cells (FIG. 3B). In contrast, knocking down p53 protein levels in the same UACC 903 cells led to no effect and an CI value of >2, which was not synergistic (FIG. 3C). To confirm these results, the effects of the drug combination were tested on two melanoma cell lines containing mutant p53 (WM164, and SK-MEL-28). As predicted, no effect on cell growth was observed when treating with the drugs and the CI values >2 showed no synergism.

Demonstration that Disrupting Reregulated MDM Protein Signaling in Melanoma Patients with Metastatic Disease Promotes Unique ICD in these Lesions Leading to an Immune Cell-Rich TME.

AKT/WEE1 inhibition in combination with anti-PD-1 treatment in syngeneic mouse melanoma models demonstrates synergistically increased anti-tumor immunity due to the inflammatory TME. Expression of chemokines that are triggered by the AKT/WEE1 inhibition-mediated ICD, such as CCL2, CXCL9, CXCL10, which can recruit immune cells that express CCR2 or CXCR3, are examined to determine the effect on tumor regression through an immune cell mechanism.

In this example, it is determined whether inhibition of AKT/WEE1 can disrupt the MDM pathway in syngeneic mouse melanoma models, leading to ICD and the activation of the TME responsive to immunotherapy. Mechanisms leading to enhanced anti-tumor immunity are identified by studying changes in the cellular composition and function of immune cells within the TME in response to inhibition of AKT/WEE1 in animals with intact immune systems and those lacking cellular components found to be increased in the TME. Immune cell populations leading to activation to promote an active TME are identified by studying the immune system cellular and cell surface interactions modulated by AKT and WEE1 and how immune cell composition as well as function change compared to MDM2/4 inhibition.

Two transplantable mouse melanoma cell lines B16.F10 (wildtype BRAF) and B16.F10-V600EBRAF are used. A further cell line, B16.F10shp53 is used to study the effect of p53 knockdown on the immunogenicity of B16F10 cells. YUMM cell lines are used to evaluate the metastasis of syngeneic tumors. Additionally, V600EBRAF-PTEN mutant SM-1 cells are used to evaluate the effect of PTEN knockdown on MDM activity.

Changes in expression, activation and transcription mediated by p53 following AKT/WEE1 inhibition are assessed to demonstrate that AKT/WEE1 inhibition can overcome the effects of high MDM2/4 levels on p53 expression, activation and transcription. AKT and WEE1 are inhibited in melanoma cells either genetically (CRISPR) or pharmacologically. Western blots of AKT, WEE1, MDM2, MDM4, p53, p-p53 and ac-p53 (Lys320) protein expression levels are then performed to show alterations in p53 protein expression in cells. To assess the level of p53 transcriptional activity, melanoma cells are transfected with a p53-Luc plasmid, which encodes for luciferase under the control of a p53 responsive element. WEE1, AKT or MDM2/4 are inhibited in cells using either CRISPR technology or pharmacological inhibitors and luciferase activity is then measured. Effects on cell cycle and apoptosis are measured along with markers such as caspase-3/9 PARP and cleaved PARP levels. Cellular proliferation is measured by MTT assay or SRB assay. RT-PCR analysis is performed for target genes transcriptionally transactivated by p53, including CDKN1A, MDM2, BBC3, BAX and APAF1. Melanoma cells are transfected with HA-ubiquitin plasmids (Addgene) followed by the knockdown of WEE1, AKT, MDM2, and MDM4 through CRISPR or pharmacologically. To dissect the p53 ubiquitination pattern, cell lysates are immunoprecipitated using G sepharose-conjugated-anti-p53 antibody (Cell Signaling) and then analyzed by western blot. Ubiquitination levels are measured using an anti-HA antibody (Thermo Fisher). Additionally, modulation of known NK-cell ligands on the cancer cells are scored across the TCGA metastatic melanoma genome dataset to define the NK cell score (infiltration) and survival of melanoma patients. Correlation between ligand score versus NK cell score and patient outcomes help identify the dominant ligand-receptor pairs that impact melanoma immunity. AKT/WEE1 inhibition reduces MDM protein-mediated inactivation of p53.

Alternative agents for pharmacological inhibition of AKT (MK2206; GDC0068, see Nitulescu, G. M., et al., Int J Oncol, 2016. 48(3): p. 869-85), MDM2 (NSC66811; SP141; RG7112, see Tisato, V., et al., J Hematol Oncol, 2017. 10(1): p.133), MDM4 (NSC207895; ALRN-6924, see Tisato, V., et al., J Hematol Oncol, 2017. 10(1): p.133), p53 (SCH529074; NSC319726, see Wang, S., et al., Cold Spring Harb Perspect Med, 2017, 7(5)), or WEE1 (PD166285; PD407824, see Matheson, C. J. et al., Trends Pharmacol Sci, 2016. 37(10): p. 872-881).

SJ-172550 binds with high affinity to MDM4 within its p53-binding pocket and disrupts the p53-MDM4 and MDM4-HIPK2 complexes (Mancini, F., et al., Oncogene, 2016, 35(2): p. 228-40). Similarly, nutlin-3 inhibits the p53-MDM2 and p53-MDM2-MDM4 complexes (Mancini, F., et al., Oncogene, 2016, 35(2): p. 228-40). Co-immunoprecipitation is used to demonstrate that inhibition of AKT/WEE1 alters the MDM2/p53, MDM2/MDM4/p53, MDM4/p53, or MDM4/HIPK2 complexes.

Melanoma cells with different genetic (p53/MDM2/MDM4) backgrounds are treated with AKT/WEE1 inhibitors and Dynabeads based magnetic separation followed by western blotting to assess interaction and binding. Co-immunoprecipitation in melanoma cell lines with various genetic backgrounds is used to assess the effect of MDM2/MDM4/p53/BRAF mutational status on the binding and interaction of the complexes. AKT/WEE1 disrupts the dissociation of the MDM2/MDM4 complex thereby reducing the MDM4/HIPK2, MDM2/p53 and MDM2/MDM4/p53 complexes similar to what occurs when targeting MDM4 with SJ-172550 or MDM2 with nutlin-3.

In addition to co-immunoprecipitation, to assess p53-MDM2/4 complexes, other techniques such as pull-down immunoassays, affinity electrophoresis, label-transfer, or chemical cross-linking of two proteins with MALDI-TOF mass spectroscopy can be used.

AKT/WEE1 inhibition modulates ICD and primes the TME to enhance the efficacy of anti-tumor immunotherapies.

Inhibition of AKT/WEE1 leads to ICD of cancer cells.

Apoptotic tumor cell death mediated by AKT/WEE1 inhibition enhances ICD as demonstrated through increases in calreticulin levels. Cell surface translocation of calreticulin, as well as p-EIF2a, ERp57, ATP and HMGB1 release are measured, and compared with the effects of MDM inhibitors on cells of the same cell lines.

AKT and WEE1 alone or in combination were administered to B16.F10 and B16.F10-BRAF cell lines and the expression of calreticulin was evaluated. B16.F10 melanoma cells were seeded at 1.5−2.0×10⁵ cells per well in 24-well plates in RPMI-1640 medium containing standard supplements and 10% FBS and incubated overnight at 37° C., 5% CO₂. Compounds solubilized in DMSO were added at the indicated concentrations and incubated for a further 24 hours at 37° C., 5% CO₂. Control wells were treated with similar dilutions of vehicle. Cells were trypsinized and washed with FACS buffer (2% FBS+0.1% NaN₃ in PBS) and seeded at 1−2×10⁵ per well in 96-well round-bottom plates. Cells were stained with rabbit anti-calreticulin polyclonal antibody (1:100, Abcam ab2907) in FACS buffer for 30 minutes at 4° C. Following two washes, cells were labeled with goat anti-rabbit Alexa Fluor 488 (1:500, Thermo Fisher A11070) in FACS buffer for 30 minutes at 4° C. Cells were washed twice and labeled with 7-aminoactinomycin D (7AAD; 0.25 μg, BD Pharmingen 51-68981E) in order to exclude non-viable cells. All samples were immediately run on a BD LSR Fortessa flow cytometer and the data analyzed using FlowJo software. Results are shown in FIG. 4 with B16.F10 cells treated with the AKT/WEE1 inhibitors show increased calreticulin levels suggesting that AKT/WEE1 targeting enhances immune cell death. These results represent one of two separate experiments showing statistically significant results.

ICD in Syngeneic Xenografts.

B16.F10 cells are subcutaneously injected into C57BL/6J mice and treated with AKT/WEE1 inhibitors or to MDM inhibitors. Tumor kinetic analysis is performed as described above, while apoptosis and p53 pathway activity are measured by western blotting and IHC. IHC is used to determine types of immune cell infiltration. Tumors are isolated, and cells profiled for calreticulin expression, ATP and HMGB1 release. RNAseq is performed on tumor tissues using CIBERSORT analysis (Newman, A. M. et al., Nat Methods, 2015. 12(5): p. 453-7) to determine the types of immune cells infiltrating the tumor and signaling (stimulatory versus suppression) occurring in this immune cell population using established IL-12/15/18-induced NK cell stimulatory gene set score versus TGFb/Activin-A/Adenosine-induced suppressive gene set scores. RNAseq analysis is used to identify changes in the different chemokines in the treated cancer cells. The role of these increased chemokines along with ICD markers to recruit immune cells into TME is evaluated by treating DCs, macrophages or NK cells in-vitro with the cytokines and evaluating their activation and migration through trans-wells. DCs and NK cells are evaluated for their immunological synapse and possible engulfment and killing of cancer cells. p53 activity is monitored for changing tumor and immune profiles during treatment. Animal groups: 5 mice X 4 treatment (vehicle, AZD5363, MK1775, AZD5363/MK1775) X 2 cell lines (B16.10, B16.F10-BRAF) X 2 sexes=80 C57BL6/J mice.

Determination of the Effects of AKT/WEE1 Inhibition on Immune Cell Populations in the TME.

The effects of inhibiting AKT/WEE1 on the immune cells of the spleen is assessed and compared to those in tumors. For this, mice with established B16.F10 or B16.F10-BRAF subcutaneous tumors (50-100 mm³) are treated daily with MK1775 and AZD5363 and compared to controls treated with MDM2/4 inhibitors. The spleen and tumors are harvested at days 7, 14 and 21 and single immune cell populations are stained and analyzed by flow cytometry. A broader analysis of the immune cell populations in the TME is performed by using the following multicolor immunostaining panels: Panel 1 to define lymphocyte subsets and their activation status: p53, MDM2/4, AKT, WEE1, CD45, NK1.1, CD19, CD3, CD4, CD8, CD25, FoxP3, PD-1, KLRG-1, CD127, CD44, fixable viability; Panel 2 to define myeloid cell populations: AKT, WEE1, p53, MDM2/4, CD45, dump gate (NK1.1, CD19, CD3), CD11b, CD11c, CD103, F480, Ly6C, Ly6G, MHC II, fixable viability; Panel 3 to define NK cell populations: AKT, WEE1, MDM2/4, CD45, CD3, NK1.1,NKp46, NKG2D, IFNγ, Granzyme B, Ki67, PD-1 and fixable viability. Immunohistochemistry (IHC) is followed by RNAseq with CIBERSORT to confirm the presence and activation of the immune cells. Sequential multi-color IHC staining is performed to show the spatial distribution of the above cells. The heterogeneous distribution of immune cells provides evidence of escape mechanisms of tumor cells. Animal groups: 12 mice X 5 groups (vehicle, AZD5363, MK1775, AZD5363/MK1775, SJ-172550/Nutlin-3) X 2 cell lines (R&R) X 2 sexes (R&R)=240 C57BL6/J mice, 4 mice per time point. AKT/WEE1 inhibition primes NK cell mediated anti-tumor immunity within the TME leading to tumor regression.

Identify the effects of AKT/WEE1 inhibition on immune cell functioning within the TME. Inhibiting AKT/WEE1 affects the function and migration potential of immune cells as can be shown by comparison of the effects on immune cells within the spleen with the effects in tumors. The function of immune cells is examined at days 7 and 14 of treatment. NK cell activity is evaluated by measuring the levels of perforin, Granzyme B, Ki67, CD69, Sca-1, FasL, TRAIL and NKG2D. NK cell effector function is measured by intracellular staining for IFNγ and TNFα following stimulation of spleen, tumor-draining lymph node or tumor-derived lymphocytes with PMA/ionomycin for 5 hours. Cytokines/chemokines such as GM-CSF, CXCL10, CXCL1, CCL3, CCL4, CCL2, IL-10 and IL-17A are evaluated by using cytokine arrays. In addition, the ability of magnetically enriched NK cells to inhibit the proliferation of CFSE-labeled CD4⁺CD25⁻effector T-cells and MDSCs is evaluated to determine the regulatory function of NK cells. The immunosuppressive function of myeloid cells to inhibit effector T-cell proliferation is also examined. Briefly, flow cytometry-sorted CD11b⁺Ly6c^{hi} monocytic and CD11b⁺Ly6c^{int}Ly6G^{hi} granulocytic cell populations are irradiated and mixed at varying ratios with 10⁵ naive CFSE-labeled CD4⁺ or CD8⁺ T-cells in the presence of PMA/ionomycin or no stimulation and loss of CFSE label is measured. Animal groups: 8 mice X 5 groups (vehicle, AZD5363, MK1775, AZD5363/MK1775, SJ-172550/Nutlin-3) X 2 cell lines X 2 sexes=160 C57BL6/J mice, 4 mice per time point. Altered NK cell activity mediated by AKT/WEE1 inhibition can be identified indicating priming of NK cell anti-tumor immunity to inhibit tumors.

In addition to B16.F10-BRAF cells forming metastases, the mouse melanoma SM1 cell line containing BRAF (V600E) with PTEN deletion (see Koya, R. C., et al., Cancer Res, 2012. 72(16): p. 3928-37) may be used. Further, metastatic YUMM1.7 cells may be used. In addition to RNAseq analysis of the immune subsets in bulk, single cell RNAseq analysis may be performed.

T-Cell Mediated Immunity is Not the Cause of Tumor Inhibition when Targeting AKT/WEE 1.

Inhibition by AKT/WEE1 in human cell lines in an immune TME lacking T-cells demonstrates that T-cells are not directly leading to inhibition of tumor cells.

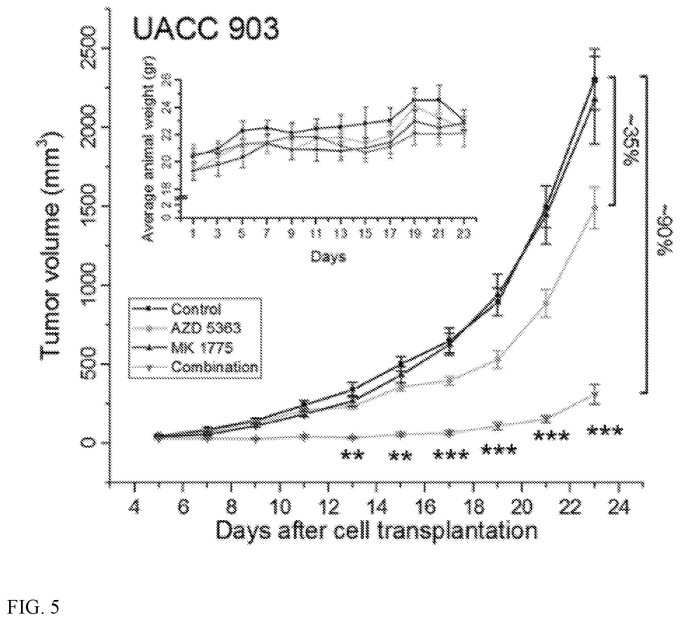

Assessing tumor development following pharmacological targeting of AKT/WEE1 in nude mice. Synergistic tumor inhibition occurs with pharmacological targeting to inhibit AKT/WEE1 in mice lacking T-cell mediated immunity. Luciferase tagged human melanoma cells are injected into nude mice to show the effect of the drug combination in mice lacking T-cells but containing NK, and dendritic cells as well as macrophages; see FIG. 5). After 7 days, mice are treated with AKT/WEE1 inhibitors and results are compared to treatment with MDM2/4 inhibitors. Blood is collected for toxicity analyses (blood glucose, ALT, AST etc.) at sacrifice. Body weight and tumor kinetics are measured on alternate days. Tumor kinetics are measured using an IVIS imaging system. Animals are sacrificed when they are moribund, have difficulty breathing, or have changes in behavioral or motor function. Markers for proliferation (Ki67), apoptosis (TUNEL) and pathway targets (p53, HIPK2, BAX, MDM2, MDM4, AKT, WEE1 and APAF1) are evaluated by IHC. Interactions of MDM2/MDM4/p53 complexes are examined as described above. Animal groups: 5 mice (two tumors/mice) X 7 groups (vehicle control, AZD5363, MK1775, AZD5363/MK1775, Nutlin-3, SJ-172550, Vem) X 4 cell lines X 2 sexes=280 athymic nude mice. Synergistic tumor inhibition results from AKT/WEE1 inhibition mediated by the disruption of MDM protein interaction with p53. AKT/WEE1 inhibition leads to synergistic inhibition of primary melanomas in nude mice, without toxicity (FIG. 5; inset shows no changes in body weight). Serum parameters also show no toxicity observed with AKT/WEE1 inhibitors.

FIG. 5 shows results of targeting of AKT/WEE1 which demonstrates that the combination inhibits melanoma tumor growth without toxicity. These results represent two separate experiments, with an N=8 for each group. ** p<0.01; ***p<0.001

Treatment of Mice and In Vivo Tumor Growth Analysis

C57BL/6J mice were purchased from The Jackson Laboratory at 6-8 weeks of age, maintained in a HEPA-filtered ventilated rack system and used within 2 weeks of receipt. Mice were housed 5 or less per cage, fed and watered ad libitum, and maintained in a 12-hour light/dark cycle. Groups of mice were inoculated with 1×10⁵ freshly cultured B16.F10 tumor cells subcutaneously in the left flank. Mice were monitored for the development of tumors and when palpable were randomized into groups. Mice received αPD-1 (clone RMP1-14; BioXCell or Leinco Technologies, Inc.) or control rat IgG (Sigma) at 200 ug/day intraperitoneally in a volume of 200 ul PBS twice per week. Mice received daily oral treatment of AZD5363 at 150 mg/kg or MK1775 at 50 mg/kg or combination of both or vehicle till the end of the experiment. Vehicle for in vivo delivery of the compounds consisted of a 0.5% methyl cellulose +0.5% Tween-80 in water. Tumors volumes and animal weights were monitored every other day. Mice were euthanized when the tumor volume exceeded 1500 mm3, developed ascites or necrosis or when mice became lethargic.

Examining the Efficacy of AKT/WEE1/Anti-PD-1 Therapy on Tumor Growth in a Syngeneic Melanoma Model.

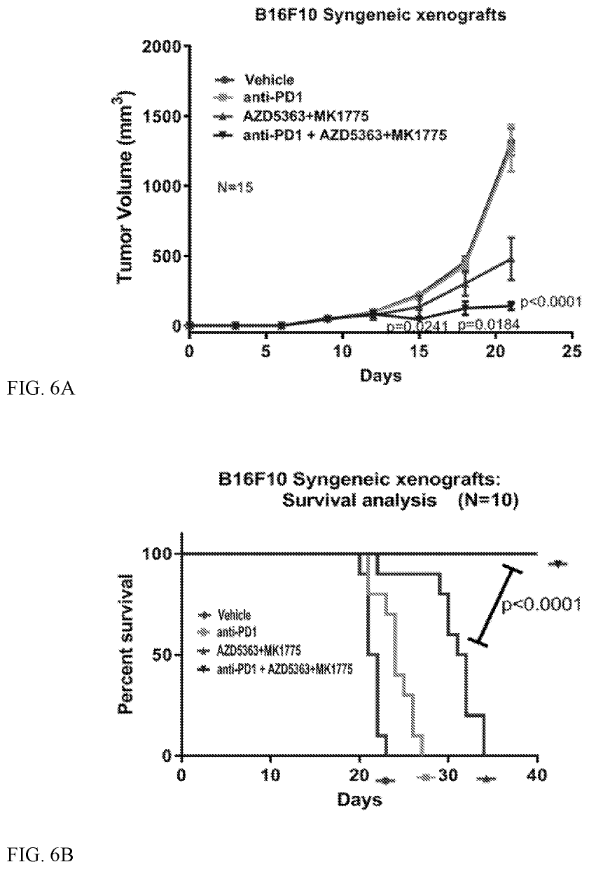

Changes in immune cell populations within the TME following AKT/WEE1 inhibition lead to enhanced efficacy of anti-PD-1 therapy. C57BL/6 mice bearing established B16.F10 or B16.F10-BRAF tumors) are treated with daily MK1775 (50 mg/kg) and AZD5363 (150 mg/kg) along with twice weekly isotype control IgG or anti-PD-1 (RMP1-14, 0.25 mg 2×/week) for 3-4 weeks (as in FIG. 6). An MDM4 inhibitor is used as a control. Tumor inhibition and the effect on mouse survival is determined. Tumor cells and infiltrating leukocytes are separated by CD45-based negative separation and individual subsets are subjected to RNAseq (detailed below). Effect of BRAF mutation on TME and on the efficacy of the combination treatment is evaluated. Animal groups: 18 mice X 6 groups (Veh+IgG, Veh+anti-PD-1, AZD5363/MK1775+IgG, AZD5363/MK1775+anti-PD-1, IgG+SJ172550, SJ172550+anti-PD-1) X 2 cell lines (B16.F10, B16.F10-BRAF) X 2 sexes=432 C57BL/6J mice (6 mice/time point sacrificed for TME, RNAseq and IHC). AKT/WEE1/anti-PD-1 therapy synergistically inhibit tumor growth.

Results in FIGS. 6A and 6B show that the combination of orally administered MK1775 and AZD5363 together with twice weekly PD-1 antibody immunotherapy was able to prevent tumor development in an immunocompetent B16F10 syngeneic mouse melanoma model.

Inhibiting MDM Pathway Signaling in Tumor Cells Decreases PD-L1 Expression to Modulate Immunogenicity.

p53 activation in tumors may increase PD-L1 expression, making the cancer cells more responsive to checkpoint inhibition. Expression of PD-L1, p53, APO-1 and FAS is measured on tumor cells and the myeloid cell population from mice containing syngeneic tumors treated with AKT/WEE1/anti-PD-1.

Immune Cell Analysis

The spleens and tumors from B16.F10 treated mice were dissected into RPMI supplemented with 10% FBS and maintained on ice. Tumors were dissociated by digesting with Colagenase-IV. Tumors and spleens were passed through a wire mesh to form a single cell suspension. Aliquots of ˜2×10⁶ cells were stained with commercially available antibodies prior to analysis on an LSR Fortessa flow cytometer (BD Biosciences) in the Penn State Cancer Institute Flow Cytometry Shared Resource. Samples were stained with fixable viability stain (FVS-AF700) prior to analysis to eliminate dead cells. Data were analyzed using FlowJo software (v10.4, FlowJo, LLC). Antibodies used include: CD45.2-BV480 (clone 104), CD4-BB700 (clone RM4-5), CD8-BV786 (clone 53-9.7), NK1.1-FITC (clone PK136), CD3-PE (clone 145-2C11), CD11c-APC-Cy7 (clone HL3), and CD11b-APC (clone M1/70), F4/80-PerCP-Cy5.5 (clone T45-2342), Ly6C-BV786 (clone AL-21), Ly6G-VF450 (clone1A8), NKG2D-BV711 (clone CX5), CD25-PE-texas Red (PC61), PD-1-PE/Dazzle (RMP1-30), FoxP3-APC (MF23). Antibodies were purchased from BD Biosciences, BioLegend, eBioscience or Tonbo Biosciences. Live cells were gated on the CD45.2+/FVS—population.

Targeting AKT/WEE1 in Syngeneic Mouse Melanoma Models to Demonstrate that the Drug Combination has Primed the Immune System in the TME Leading to an Effective Immune Response with Anti-PD-1.

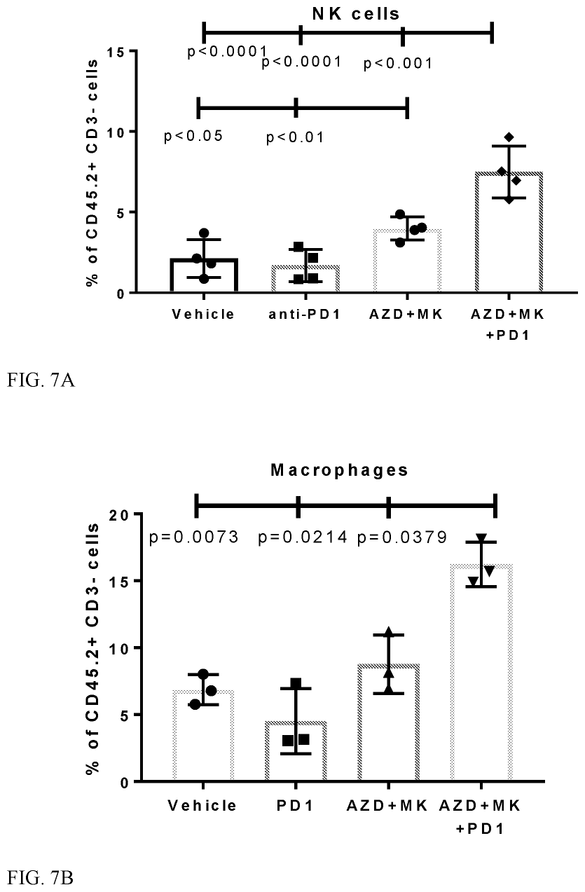

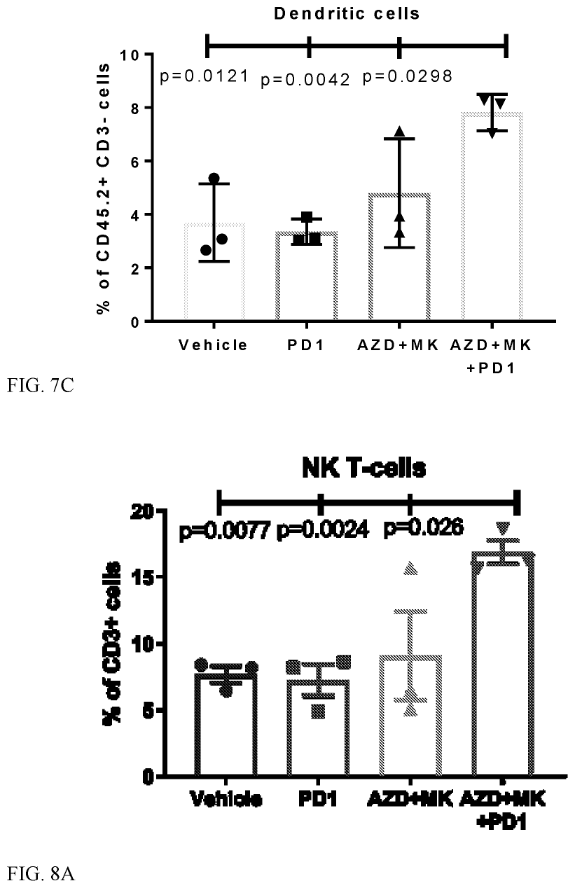

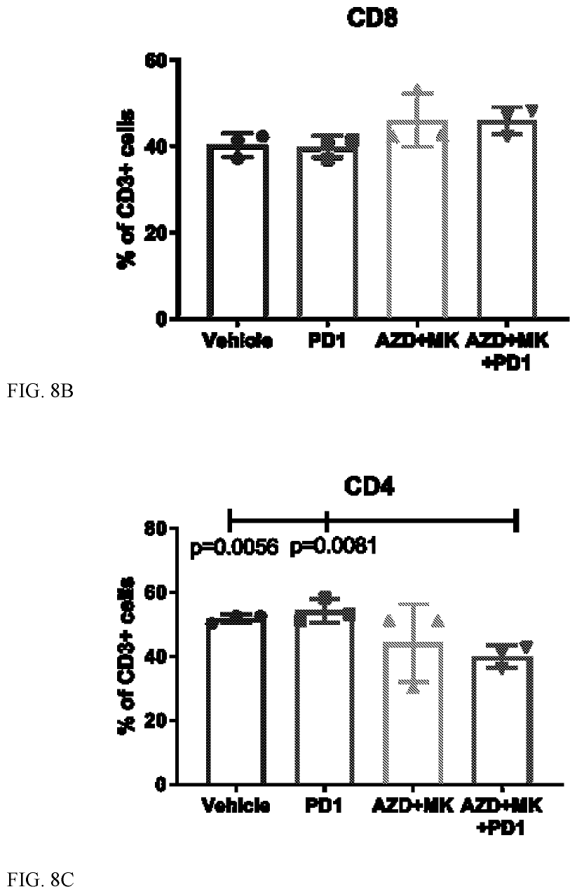

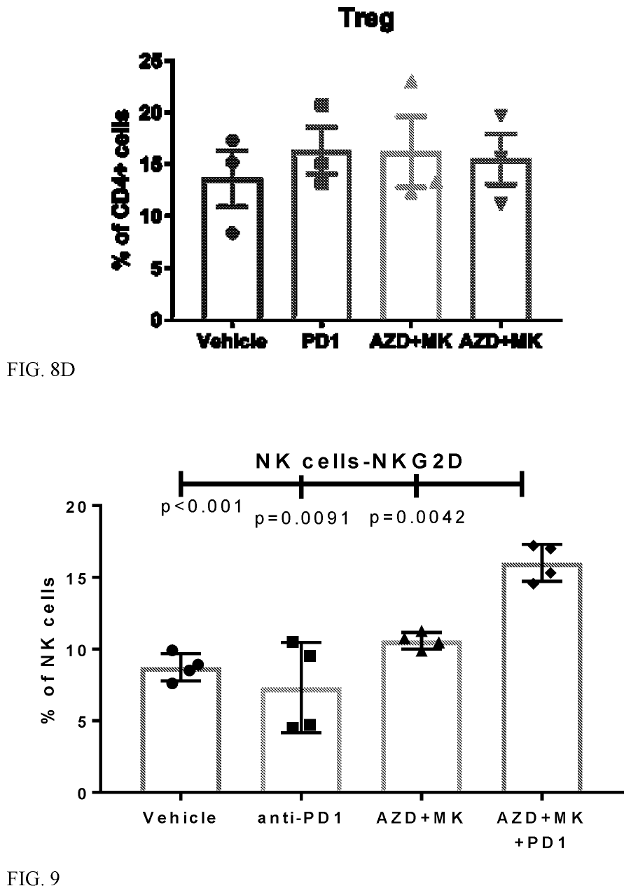

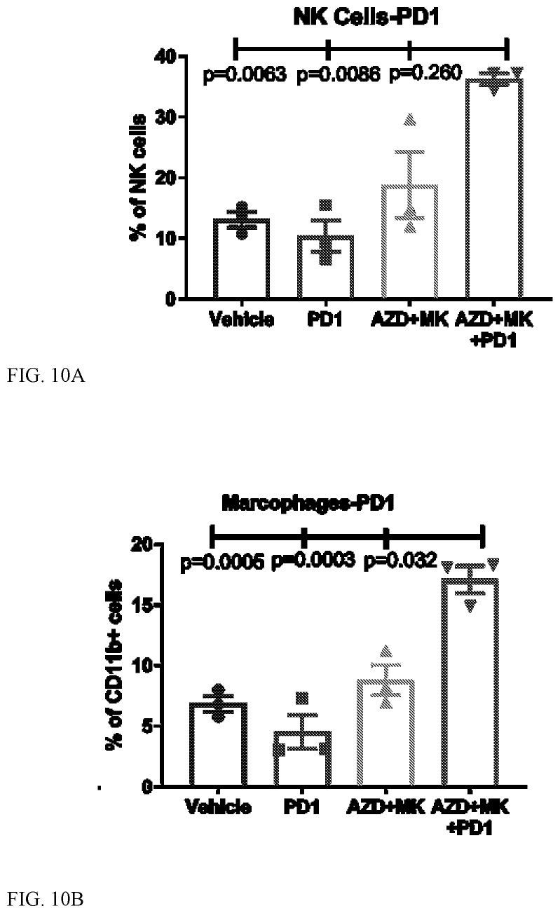

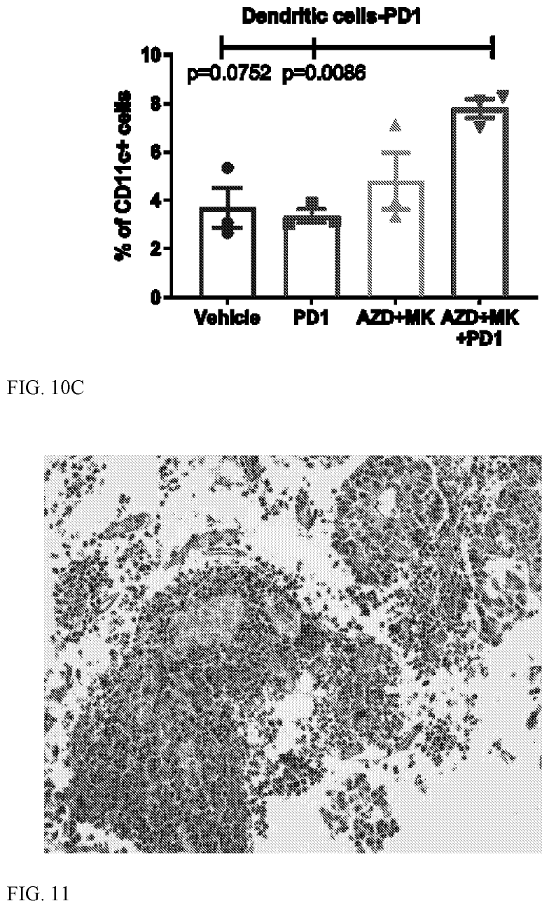

The combination of orally administered MK1775 and AZD5363 together with PD-1 antibody immunotherapy was able to prevent subcutaneous tumor development in an immunocompetent B16.F10 syngeneic mouse melanoma model. Immune cell analysis of the TME indicated a significant increase in NK cells, macrophages and dendritic cells (DCs) in the triple combination treatment compared to vehicle control or any of the individual treatments suggesting the strong influence of innate immunity with the drug combination (FIGS. 7A-C). Additionally, the levels of NK T-cells significantly increased in the TME without any significant changes in CD4+ or CD8+ T-cells or T_{regs} (FIGS. 8A-D). Specifically, the triple combination significantly increased NK recruitment (FIGS. 7A-C) and function within the TME (FIG. 9). NKG2D, a strong activating receptor on NK cells was significantly increased. Additionally, a dose dependent increase in Granzyme B, Ki67 and IFNγ production in NK cells was observed. Moreover, the levels of PD-1 expression increased on NK cells, macrophages and DCs in the triple combination. (FIGS. 10A-C). IHC (FIG. 11) and preliminary RNAseq analysis are used to confirm the recruitment of NK cells in tumors.

The results show a significant increase in NK cells (FIG. 7A), macrophages (FIG. 7B), and dendritic cells (DCs) (FIG. 7C), in the triple combination treatment (AKT inhibitor/WEE1 inhibitor/PD-1 inhibitor) compared to vehicle control or any of the individual treatments shows the strong influence of innate immunity with the drug combination. Additionally, the levels of NK T-cells significantly increased in the tumor microenvironment (TME) (FIG. 8A) without any significant changes in CD8+ (FIG. 8B) or CD4+ (FIG. 8C) T-cells or Tregs (FIG. 8D). Specifically, the triple combination (AKT inhibitor/WEE1 inhibitor/PD-1 inhibitor) significantly increased NK recruitment and function within the TME (FIG. 9). NKG2D, the cell killing receptor of NK cells was significantly increased. Additionally, a dose dependent increase in Granzyme B, Ki67 and IFNγ production in NK cells was observed. Moreover, the levels of PD-1 expression increased on NK cells (FIG. 10A), macrophages (FIG. 10B), and DCs (FIG. 10C), in the triple combination (AKT inhibitor/WEE1 inhibitor/PD-1 inhibitor). Finally, there were no changes in immune cell subsets within the spleens. A possible explanation for high NK cell activity but not T-cell based immunity is probably due to the pronounced effect of WEE1 inhibition on NK-cell activity and function. Tumor cell resistance to granzyme B-induced cell death can be reversed through inhibition of WEE1 kinase as AZD1775 sensitized both murine and human head and neck cancer cells to NK lysis. Note: IHC studies (FIG. 11) and RNAseq analysis confirmed the recruitment of NK cells in tumors.

Examination of Changes in immune cell composition within the TME following AKT/WEE1/anti-PD-1 therapy. Immune cell population changes within the TME are identified by this method following combination therapy. AKT/WEE1/anti-PD-1 therapy leads to increased NK cell-mediated anti-tumor immunity (FIGS. 7-9).

Inhibiting AKT/WEE leads to an increase in p53 levels and/or activity and upregulation of NKG2D ligands on tumor cells causing increased tumor recognition and destruction.

Inhibiting the MDM pathway leads to an increase in p53 pathway activity and tumor cell release of cytokines, such as interleukin (IL)-12, IL-15 and IL-18, functionally activating NK cells, increasing anti-cancer immunity mediated by NK cells.

Flow cytometric staining panels described in are used to define the immune cell populations within the same 9 treatment groups at days 7, 14 and 21. This includes evaluation of AKT and WEE1, as well as p53 and MDM2/4 levels/activity in these cell subsets and analysis of NK cell activation, function and immune suppressive function by T_{regs} and myeloid cells. Similarly, RNA seq, FNA and IHC staining are performed as described above. Specifically, IHC changes to the TME including changes to any of CD4, CD8, FoxP3, CD11c, CD11b, NKp46, NK1.1 and CD103 are evaluated in tumor sections from representative mice. Whole tumors, separated tumor cells and TILs as described above are subjected to RNAseq analysis to evaluate NK cell and myeloid cell activation/signaling. Expression of cellular markers for NK cell and myeloid cell function such as NKG2D, CD27, PD-1, CD16/32, Ly6C, Ly6G and F4/80 are evaluated. Cytokines/chemokines important in activation, recruitment and function of NK cells and accessory cells, such as XCL1, CCL5, CCL2, CCL3, CCL4, CXCL1, CXCL10, GMCSF, TGFβ and IL-10, are evaluated. Expression levels of proteins modulating the p53 pathway such as MDM2/4, p53, AKT, WEE1 and the effector molecules of the p53 pathway such as CDK1, p21, p27, H2AX are evaluated. Activation of other immunological pathways that represent crosstalk and resistance to PD-1 targeted therapies are evaluated. The effect of AKT/WEE1/PD-1 inhibition on other immunological pathways such as LAG3, TIM3, TIGIT, 4-1BB, OX40 and CTLA-4 is evaluated.

Demonstrating the involvement of a specific immune cell population in the TME.

The role of immune cells populations identified and infiltrating and being activated in the TME in response to AKT/WEE1/PD-1 therapy is demonstrated through immune cell depletion and repletion studies.

One of: NK cells, CD8 T cells, CD11b+ or CD11c+ cells is depleted from mice prior to the administration of AKT/WEE1. Groups of C57BL/6 mice are challenged with B16.F10 tumors and once established, mice are depleted of NK cells by the injection of either anti-NK1.1 (clone PK163), anti-CD8 (clone YTS 169.4), myeloid cells (anti-CD11b; clone M1/70) or dendritic cells anti-CD115 (AFS98) or their combination at day 2 prior to initiation of AKT/WEE1 therapy. Depletion is maintained by weekly antibody injections and AKT/WEE1 continued for 3 weeks. Anti-PD-1 antibody is administered. Depletion of the indicated cells is confirmed by monitoring immune cells in peripheral blood and representative tumors. Mice are monitored for tumor growth and the survival of mice determined as above. Animal groups: 5 mice X 4 groups (anti-CD11b, anti-CD11c, anti-NK1.1, control Ab) X 1 treatment (AZD5363/MK1775/anti-PD-1) X 2 sexes (R&R)=40 C57BL6/J mice.

Effects of AKT/WEE1 treatment on metastasis as well as primary tumors-comparison of pharmacological AKT/WEE1 inhibition with MDM2/4 inhibitors for targeting spontaneous melanoma metastasis.

A spontaneous metastasis model with GFP-tagged mouse melanoma cell lines is used. Mice are treated with pharmacological inhibitors of AKT/WEE1 and MDM2/4 when the primary tumor is palpable. Body weight and primary tumor dimensions are measured on alternate days. Animals are sacrificed on day 40 to examine metastases by fluorescent microscopy/IHC. Appropriate controls such as vehicle or Vemurafenib are included. Interactions between MDM2/4 and p53 are examined as described above. Markers for proliferation, apoptosis and upstream/downstream targets are evaluated by IHC and western blot. The TME is evaluated as in described above. Blood is collected for toxicity analyses at sacrifice. 20 mice (2 sexes) X 7 groups (vehicle control, AZD5363, MK1775, AZD5363/MK1775, Nutlin-3, SJ-172550, Vemurafenib)=140 C57BL/6J mice.

Assessing metastasis following administration of a combination of AKT/WEE1/anti-PD-1 inhibition. Combination of AKT/WEE1/PD-1 inhibition is evaluated in a spontaneous metastasis model with GFP-tagged mouse melanoma cell lines. Mice are treated with pharmacological inhibitors of AKT/WEE1 and an anti-PD-1 antibody when the primary tumor is palpable. Body weight and primary tumor dimensions are measured on alternate days. Animals are sacrificed on day 40 to examine metastases by fluorescent microscopy/IHC. Appropriate controls such as vehicle or Vemurafenib are included. Interactions between MDM2/4 and p53 are examined as described above. Markers for proliferation, apoptosis and upstream/downstream targets are evaluated by IHC and western blot. The TME is evaluated as in described above. Blood is collected for toxicity analyses at sacrifice. Animals required: 20 mice (2 sexes) X 5 groups (vehicle control, AZD5363/MK1775, anti-PD-1, anti-PD-1+AZD5363/MK1775, Vemurafenib)=100 C57BL/6J mice.

Efficacy in patient derived metastasis xenografts (PDX) that are wild type for p53 but not for those mutant for the protein.

The effect of p53 modulation on the NK cell ligands in PDX cells is first evaluated in-vitro. PDX models that are p53 wildtype and express high levels of MDM2/4 are used. Six days after subcutaneous injection of the melanoma cells, mice are treated with AKT/WEE1 inhibitors and tumor development assessed. For comparison, mice are treated with MDM2/4 inhibitors. Mice are sacrificed after 30-60 days depending on the tumorigenic and/or metastatic potential of the PDX cells. Markers for proliferation, apoptosis and upstream/downstream targets are evaluated by IHC and western blot. Animal groups: 5 mice/group X 8 treatment groups (vehicle, AZD5363, MK1775, AZD5363/MK1775, SJ172550, Nutlin-3, Vem, AZD5363/MK1775/Vem) X 4 PDX (2 BRAF mutant & 2 BRAF wild type) X 2 sexes=320 NSG mice.

Disrupting the deregulated MDM pathways in melanoma leads to unique recruitment and activation of NK cells in the TME promoting NK cell mediated immunity.

Inhibiting AKT/WEE1 to inhibit the MDM protein pathways can be used to activate NK cell-mediated anti-tumor immunity both by antigens generated from the killed melanoma cells and by activating the NK cells themselves. This is demonstrated by determining that MDM activity within tumor cells can modulate NK cell tumor immune-surveillance and subsequently anti-tumor immunity. MDM inhibition increases the immunogenicity through production of chemokines by tumor cells, such as CCL2, CXCL9/10, which recruit NK cells to the TME. p53 activity increases the production of cytokines important for NK cell activation, such as IL-12, IL-15 and IL-18. p53 expression by tumor cells can upregulate NKG2D ligands, particularly ULBP1 and ULBP2, leading to increased recognition and the destruction by NK cells. NK cells are depleted to evaluate the effect of NK-cell mediated immunity on the response of p53 modulation.

Increasing p53 pathway activity by inhibiting AKT/WEE leads to upregulation of NKG2D ligands on tumor cells causing increased tumor recognition and destruction.

NK cell activity following AKT/WEE1/anti-PD-1 is analyzed first by isolating NK cells from animals treated with the combination of AKT/WEE1/anti-PD-1 and evaluating the cytotoxic activity of NK cells on cultured cancer cells. KIL cells serve as positive controls. NK cells are isolated via negative bead separation from splenocytes and tumors. The GFP tagged mouse melanoma cell lines B16.F10 and B16.F10-BRAF (either treated or untreated) are incubated with treated or untreated mouse NK cells and the amount of live cancer cells is estimated at different effector-to-target ratios. The ability of NK cells to kill the cancer cells is evaluated in vitro and in vivo by incorporating the Annexin-V-PE analysis on the cancer cells. Moreover, cytokines responsible for activation, recruitment and function of NK cells, such as IL-12, IL-18, IL-15, CCL2, CXCL9/10, TNFα and IFNγ, are evaluated by ELISA or RNAseq analysis. Granzyme B, Perforin, FasL and Trail are monitored by FACS. The identified mechanism is confirmed by blocking the function of the relevant cytokines/death receptor ligands with antibodies. Animal groups: 5 mice X 4 groups (vehicle control, AZD5363/MK1775, NK cell therapy, AZD5363/MK1775/anti-PD-1+NK cell therapy) X 2 sexes=40 C57BL6/J mice.

Increasing p53 pathway activity by targeting AKT/WEE leads to tumor cell release of cytokines, such as interleukin (IL)-12, IL-15 and IL-18, to functionally activate NK cells, increasing anti-cancer immunity mediated by NK cells.

B16.F10 cells are treated with MDM pathway inhibitors and evaluated for the release of IL12, IL15 and IL18 using individual, commercially available ELISA kits.

Demonstration of increased NK-cell-mediated killing of tumor cells following MDM pathway inhibition. In vitro killing assays on human and mouse tumor lines post-MDM inhibition are performed to monitor immunogenicity. The immunogenicity of human and mouse melanoma cells post-MDM inhibition is evaluated using the NK ligand score (detailed in Cursons, J., et al., Cancer Immunol Res, 2019, 7(7): p. 1162-1174), a measure of NK cell infiltration and survival of melanoma patients. As positive controls, PARP or CDK4/6 inhibitors are used. Additionally, an increase in NK cell activity is evaluated by incubating CFSE-tagged or GFP-tagged cancer cells with NK cells at different effector-to-target ratios. The interaction is evaluated by the decrease in fluorescence due to the killing of cancer cells (as detailed in Hennessy, R. J., et al., J Leukoc Biol, 2019, 105(6): p. 1341-1354). The effect of BRAF/PTEN/MDM/P53 mutations on the immunogenicity and elimination of cancer cells by NK cells is evaluated. Similarly, patient tumors are incubated with NK-cells isolated from the same patient in the presence of MDM inhibitors for clinical relevance.

NK cell infiltration and activation following AKT/WEE1/anti-PD-1 treatment.

Ncr1-GFP mice contain GFP-tagged NK cells and are used to monitor NK-cells as detailed in Kerdiles, Y. M., et al., Immunity, 2017, 47(2): p. 199-200. Mice are challenged with B16.F10 tumors and once established, mice are treated with AKT/WEE1/anti-PD-1 combinatinon therapy. Mice are monitored for tumor growth and the survival determined as above. Similarly, Ncr1 KO mice are used since Ncr1 expression is required for NK cell-derived IFNγ production in some murine melanoma models, see Glasner, A., et al., Immunity, 2018, 48(1): p. 107-119 e4. NK cell survival and proliferation are measured as described previously in Viant, C., et al., J Exp Med, 2017, 214(2): p. 491-510. At the end of the experiment, tumors are collected for IHC, RNAseq and flow cytometry. NK-cell activation is confirmed by measuring intracellular IFNγ and GzmB along with activating receptors along with their corresponding ligands on the tumors as also described in Viant, C., et al., J Exp Med, 2017, 214(2): p. 491-510. Animal groups: 10 mice X 2 treatment (vehicle, AZD5363/MK1775/anti-PD-1) X 2 sexes=40 Ncr1-GFP mice.

NK cells in the TME following AKT/WEE1/anti-PD-1 therapy inhibits tumors.

The role of NK cells in the TME in response to AKT/WEE1/anti-PD-1 therapy is demonstrated using NK cell depletion and repletion studies.

NK cells are conditionally deleted in B6 mice using a Mcl1.1oxP Ncr1-cre model detailed in Sathe, P., et al., Nat Commun, 2014, 5: p. 4539.

Specific deletion of Mcl1 in NK cells results in the absolute loss of NK cells from all tissues due to failure to antagonize pro-apoptotic proteins in the outer mitochondrial membrane. Knock-down is confirmed by monitoring NK cells in peripheral blood and representative tumors. Mice are challenged with B16.F10 tumors and once established, mice are treated with AKT/WEE1/anti-PD-1 therapy. Mice are monitored for tumor growth and the survival determined as above. Animal groups: 10 mice X 2 treatment (vehicle, AZD5363/MK1775/anti-PD-1) X 2 sexes=40 Mcl1.1oxP Ncr1-cre mice.

Items

Item 1. A method of treating cancer in a subject in need thereof, including: administering a combination of: 1) an anti-cancer immune therapeutic agent, and 2) a stimulator of p53 levels and/or p53 activity, as a combination formulation or separately.

Item 2. The method of treating cancer of item 1, wherein the cancer is characterized by wild-type p53 expression.

Item 3. The method of item 1 or 2, wherein the anti-cancer immune therapeutic agent includes a cell-based agent or a non-cell based agent.

Item 4. The method of item 1, 2 or 3, wherein the anti-cancer immune therapeutic agent includes a cell-based agent selected from: an NK cell-based anti-cancer immune therapeutic agent; and a CAR-T cell-based anti-cancer immune therapeutic agent.

Item 5. The method of any of items 1 to 4, wherein the anti-cancer immune therapeutic agent includes an immune checkpoint inhibitor.

Item 6. The method of any of items 1 to 5, wherein the anti-cancer immune therapeutic agent includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR).

Item 7. The method of any of items 1 to 6, wherein the stimulator of p53 levels and/or p53 activity inhibits degradation of p53.

Item 8. The method of any of items 1 to 7, wherein the stimulator of p53 levels and/or p53 activity includes an inhibitor of MDM2 and/or MDM4.

Item 9. The method of any of items 1 to 8, wherein the stimulator of p53 levels and/or p53 activity includes an inhibitor of AKT and an inhibitor of WEE1, wherein the inhibitor of AKT and an inhibitor of WEE1 are administered together as a combination formulation or separately.

Item 10. The method of any of items 1 to 9, wherein administration of the combination provides a synergistic effect, thereby treating the cancer.

Item 11. The method of any of items 1 to 10, wherein administration of the combination stimulates immunogenic tumor cell death of cancer cells of the cancer.

Item 12. The method of any of items 1 to 11, wherein administration of the combination stimulates recruitment of NK cells to the cancer and/or activates NK cells in the cancer.

Item 13. The method of item 9, wherein the AKT inhibitor includes an AKT inhibitor selected from the group consisting of: AZD5363, GDC0068, a combination of

AZD5363 and GDC0068, a pharmaceutically acceptable salt, hydrate, amide or ester of any thereof, an siRNA directed to AKT, and a CRISPR/Cas knockdown construct directed to AKT.

Item 14. The method of item 9, wherein the WEE1 inhibitor includes a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Item 15. The method of treating cancer of any of items 1 to 14, wherein the anti-cancer immune therapeutic agent includes an immune checkpoint inhibitor selected from the group consisting of: a PD-1 inhibitor, a PD-1 ligand (PD-L1) inhibitor, and a CTLA4 inhibitor.

Item 16. The method of treating cancer of any of items 1 to 15, wherein the anti-cancer immune therapeutic agent includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4.

Item 17. The method of treating cancer of any of items 1 to 16, wherein the cancer is characterized by constitutive activation of a mitogen-activated protein kinase-signaling pathway.

Item 18. The method of treating cancer of any of items 1 to 17, wherein the cancer is characterized by constitutive activation of a mitogen-activated protein kinase-signaling pathway associated with one or more mutations in BRAF, KIT and/or RAS.

Item 19. The method of treating cancer of any of items 1 to 18, wherein the cancer is characterized by constitutive activation of a mitogen-activated protein kinase-signaling pathway associated with ^{V600E}BRAF.

Item 20. The method of treating cancer of any of items 1 to 19, wherein the cancer is characterized by AKT dysregulation.

Item 21. The method of treating cancer of any of items 1 to 20, wherein the cancer is selected from the group consisting of: melanoma, colorectal cancer, thyroid cancer, breast cancer, prostate cancer, sarcoma, glioblastoma, T-cell acute lymphoblastic leukaemia, lung cancer and liver cancer.

Item 22. The method of treating cancer of any of items 1 to 21, wherein the cancer is melanoma.

Item 23. The method of treating cancer of any of items 1 to 22, further including: obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for one or more markers of apoptosis, thereby monitoring effectiveness of administering the combination.

Item 24. The method of treating cancer of any of items 1 to 23, further including: obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for activity of a mitogen-activated protein kinase-signaling pathway, thereby monitoring effectiveness of administering the combination.

Item 25. The method of treating cancer of any of items 1 to 24, further including: obtaining a first sample containing or suspected of containing cancer cells from the subject prior to administering the combination of the combination of the anti-cancer immune therapeutic agent and the stimulator of p53 levels and/or p53 activity; obtaining a second sample containing or suspected of containing cancer cells from the subject after administering the combination; and assaying the first and second samples for AKT dysregulation, thereby monitoring effectiveness of administering the combination.

Item 26. The method of treating cancer of any of items 1 to 25, further including: obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for p53 expression and/or an associated gene selected from the group consisting of: CLCA2, PVRL4, SULF2, CDKN1a, BTG2, ACTA2, TP5363, FDXR, GDF15, IGFBP5 and ADAM19, wherein an increase in p53 expression and/or expression of an associated gene selected from the group consisting of: CLCA2, PVRL4, SULF2, CDKN1a, BTG2, ACTA2, TP5363, FDXR, GDF15, IGFBP5 and ADAM19, is an indicator of an anti-cancer cell effect of treatment with the combination, thereby monitoring effectiveness of administering the combination.

Item 27. The method of treating cancer of any of items 1 to 26, further including: obtaining a first sample containing or suspected of containing cancer cells from the subject prior to administering the combination of the anti-cancer immune therapeutic agent and the stimulator of p53 levels and/or p53 activity; obtaining a second sample containing or suspected of containing cancer cells from the subject after administering the combination; and assaying the first and second samples for FOXM1 expression and/or expression of an associated gene selected from the group consisting of: TMPO, ANP32E, SMC4, KIF20B, ASPM, DEPDC1, NCAPG, CENPE, wherein a decrease in expression of FOXM1 and/or an associated gene selected from the group consisting of: TMPO, ANP32E, SMC4, KIF20B, ASPM, DEPDC1, NCAPG and CENPE, is an indicator of an anti-cancer cell effect of treatment with the combination, thereby monitoring effectiveness of administering the combination.

Item 28. The method of treating cancer of any of items 1 to 27, wherein the anti-cancer immune therapeutic agent and the stimulator of p53 levels and/or p53 activity are administered simultaneously.

Item 29. The method of treating cancer of any of items 1 to 28, wherein all of: the anti-cancer immune therapeutic agent and the stimulator of p53 levels, are administered sequentially.

Item 30. The method of treating cancer of any of items 1 to 29, wherein the stimulator of p53 levels and/or p53 activity includes an AKT inhibitor and a WEE1 inhibitor, and wherein: 1) the anti-cancer immune therapeutic agent and the AKT inhibitor are administered simultaneously and the WEE1 inhibitor is administered sequentially with respect to the anti-cancer immune therapeutic agent and the AKT inhibitor; 2) the anti-cancer immune therapeutic agent and the WEE1 inhibitor are administered simultaneously and the AKT inhibitor is administered sequentially with respect to the anti-cancer immune therapeutic agent and the WEE1 inhibitor; 3) the WEE1 inhibitor and the AKT inhibitor are administered simultaneously and the anti-cancer immune therapeutic agent is administered sequentially with respect to the WEE1 inhibitor and the AKT inhibitor; or wherein all of: the anti-cancer immune therapeutic agent, the AKT inhibitor, and the WEE1 inhibitor, are administered sequentially.

Item 31. The method of treating cancer of any of items 1 to 30, wherein the stimulator of p53 levels and/or p53 activity includes an MDM2 and/or MDM4 inhibitor, and wherein: 1) the anti-cancer immune therapeutic agent and the MDM2 and/or MDM4 inhibitor are administered simultaneously; or 2) the anti-cancer immune therapeutic agent and the MDM2 and/or MDM4 inhibitor are administered sequentially.

Item 32. The method of treating cancer of any of items 29 to 31, wherein administered sequentially refers to administration within a period of time selected from: one hour, two hours, four hours, eight hours, twelve hours, twenty-four hours, 2 days, 3 days, 4 days, 5 days, 6 days and 7 days.

Item 33. The method of treating cancer of any of items 1 to 32, wherein the subject is human.

Item 34. The method of treating cancer of any of items 1 to 33, wherein p53 is increased in the subject following administration of the composition.

Item 35. The method of any of items 1-34 wherein the AKT inhibitor includes an AKT3 inhibitor.

Item 36. A commercial package, including: 1) an anti-cancer immune therapeutic agent, and 2) a stimulator of p53 levels and/or p53 activity.

Item 37. The commercial package of item 36, wherein the anti-cancer immune therapeutic agent includes a cell-based agent or a non-cell based agent.

Item 38. The commercial package of item 36 or 37, wherein the anti-cancer immune therapeutic agent includes a cell-based agent selected from: an NK cell-based anti-cancer immune therapeutic agent; and a CAR-T cell-based anti-cancer immune therapeutic agent.

Item 39. The commercial package of any of items 36 to 38, wherein the anti-cancer immune therapeutic agent includes an immune checkpoint inhibitor.

Item 40. The commercial package of any of items 36 to 39, wherein the anti-cancer immune therapeutic agent includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR).

Item 41. The commercial package of any of items 36 to 40, wherein the stimulator of p53 levels and/or p53 activity inhibits degradation of p53.

Item 42. The commercial package of any of items 36 to 41, wherein the stimulator of p53 levels and/or p53 activity includes an inhibitor of MDM2 and/or MDM4.

Item 43. The method of any of items 36 to 42, wherein the stimulator of p53 levels and/or p53 activity includes an inhibitor of AKT and/or an inhibitor of WEE1.

Item 44. The commercial package of item 43, wherein the AKT inhibitor includes an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT.

Item 45. The commercial package of item 43, wherein the WEE1 inhibitor includes a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Item 46. The commercial package of any of items 36 to 45, wherein the anti-cancer immune therapeutic agent includes an immune checkpoint inhibitor selected from the group consisting of: a PD-1 inhibitor, a PD-1 ligand (PD-L1) inhibitor, and a CTLA4 inhibitor.

Item 47. The commercial package of any of items 36 to 46, wherein the anti-cancer immune therapeutic agent includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4.

Item 48. The commercial package of any of items 36-47 wherein the AKT inhibitor includes an AKT3 inhibitor.

Item 49. The commercial package of any of items 36-48 wherein the commercial package excludes a CHK1 inhibitor and/or an mTOR inhibitor.

Item 50. A composition, including: 1) an anti-cancer immune therapeutic agent, and 2) a stimulator of p53 levels and/or activity.

Item 51. The composition of item 50, wherein the anti-cancer immune therapeutic agent includes a cell-based agent or a non-cell based agent.

Item 52. The composition of item 50 or 51, wherein the anti-cancer immune therapeutic agent includes a cell-based agent selected from: an NK cell-based anti-cancer immune therapeutic agent; and a CAR-T cell-based anti-cancer immune therapeutic agent.

Item 53. The composition of any of items 50 to 52, wherein the anti-cancer immune therapeutic agent includes an immune checkpoint inhibitor.

Item 54. The composition of any of items 50 to 53, wherein the anti-cancer immune therapeutic agent includes a non-cell based agent selected from: an indoleamine 2,3-dioxygenase 1 (IDO1) inhibitor, a lymphocyte-activation gene 3 (LAG3) antibody, a T-cell immunoglobulin and mucin domain-3 (TIM3) antibody, an OX-40 agonist, and a Glucocorticoid-induced TNFR-related (GITR).

Item 55. The composition of any of items 50 to 54, wherein the stimulator of p53 levels and/or activity inhibits degradation of p53.

Item 56. The composition of any of items 50 to 55, wherein the stimulator of p53 levels and/or activity includes an inhibitor of MDM2 and/or MDM4.

Item 57. The method of any of items 50 to 56, wherein the stimulator of p53 levels and/or activity includes an inhibitor of AKT and/or an inhibitor of WEE1.

Item 58. The composition of item 57, wherein the AKT inhibitor includes an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT.

Item 59. The composition of item 57, wherein the WEE1 inhibitor includes a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

Item 60. The composition of treating cancer of any of items 50 to 59, wherein the anti-cancer immune therapeutic agent includes an immune checkpoint inhibitor selected from the group consisting of: a PD-1 inhibitor, a PD-1 ligand (PD-L1) inhibitor, and a CTLA4 inhibitor.

Item 61. The composition of treating cancer of any of items 50 to 60, wherein the anti-cancer immune therapeutic agent includes an immune checkpoint inhibitor selected from the group consisting of: nivolumab, pembrolizumab, atezolizumab, avelumab, durvalumab, ipilimumab, an siRNA directed to PD-1, PD-L1, or CTLA4, and a CRISPR/Cas knockdown construct directed to PD-1, PD-L1, or CTLA4.

Item 62. The composition of any of items 50 to 61, further including a pharmaceutically acceptable carrier.

Item 63. The composition of any of items 50 to 62, wherein the AKT inhibitor includes an AKT3 inhibitor.

Item 64. The composition of any of items 50 to 63, wherein the composition excludes a CHK1 inhibitor and/or an mTOR inhibitor.

Item 65. A method of treating cancer in a subject substantially as described herein.

Item 66. A composition substantially as described herein.

Item 67. A pharmaceutical composition substantially as described herein

Item 68. A commercial package substantially as described herein.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treating cancer in a subject in need thereof, comprising:
administering a combination of: 1) an anti-cancer immune therapeutic agent, and 2) a stimulator of p53 levels and/or p53 activity, as a combination formulation or separately.

2. The method of treating cancer of claim 1, wherein the cancer is characterized by wild-type p53 expression.

3. The method of claim 1, wherein the anti-cancer immune therapeutic agent comprises a cell-based agent or a non-cell based agent.

4. The method of claim 1, wherein the anti-cancer immune therapeutic agent comprises a cell-based agent selected from: an NK cell-based anti-cancer immune therapeutic agent; and a CAR-T cell-based anti-cancer immune therapeutic agent.

5. The method of claim 1, wherein the anti-cancer immune therapeutic agent comprises an immune checkpoint inhibitor.

6.-8. (canceled)

9. The method of claim 1, wherein the stimulator of p53 levels and/or p53 activity comprises an inhibitor of AKT and an inhibitor of WEE1, wherein the inhibitor of AKT and an inhibitor of WEE1 are administered together as a combination formulation or separately.

10 The method of claim 1, wherein administration of the combination provides a synergistic effect, thereby treating the cancer.

11. The method of claim 1, wherein administration of the combination stimulates immunogenic tumor cell death of cancer cells of the cancer.

12. The method of claim 1, wherein administration of the combination stimulates recruitment of NK cells to the cancer and/or activates NK cells in the cancer.

13. The method of claim 9, wherein the AKT inhibitor comprises an AKT inhibitor selected from the group consisting of: AZD5363, GDC0068, a combination of AZD5363 and GDC0068, a pharmaceutically acceptable salt, hydrate, amide or ester of any thereof, an siRNA directed to AKT, and a CRISPR/Cas knockdown construct directed to AKT.

14. The method of claim 9, wherein the WEE1 inhibitor comprises a WEE1 inhibitor selected from the group consisting of: MK1775, a pharmaceutically acceptable salt, hydrate, amide or ester thereof, an siRNA directed to WEE1, and a CRISPR/Cas knockdown construct directed to WEE1.

15. The method of treating cancer of claim 1, wherein the anti-cancer immune therapeutic agent comprises an immune checkpoint inhibitor selected from the group consisting of: a PD-1 inhibitor, a PD-1 ligand (PD-L1) inhibitor, and a CTLA4 inhibitor.

16. (canceled)

17. The method of treating cancer of claim 1, wherein the cancer is characterized by constitutive activation of a mitogen-activated protein kinase-signaling pathway.

18. The method of treating cancer of claim 1, wherein the cancer is characterized by constitutive activation of a mitogen-activated protein kinase-signaling pathway associated with one or more mutations in BRAF, KIT and/or RAS.

19. The method of treating cancer of claim 1, wherein the cancer is characterized by constitutive activation of a mitogen-activated protein kinase-signaling pathway associated with ^{V600E}BRAF.

20. The method of treating cancer of, wherein the cancer is characterized by AKT dysregulation.

21. The method of treating cancer of claim 1, wherein the cancer is selected from the group consisting of: melanoma, colorectal cancer, thyroid cancer, breast cancer, prostate cancer, sarcoma, glioblastoma, T-cell acute lymphoblastic leukaemia, lung cancer and liver cancer.

22.-27. (canceled)

28. The method of treating cancer of claim 1, wherein the anti-cancer immune therapeutic agent and the stimulator of p53 levels and/or p53 activity are administered simultaneously.

29. The method of treating cancer of claim 1, wherein all of: the anti-cancer immune therapeutic agent and the stimulator of p53 levels, are administered sequentially.

30. The method of treating cancer of claim 1, wherein the stimulator of p53 levels and/or p53 activity comprises an AKT inhibitor and a WEE1 inhibitor, and wherein: 1) the anti-cancer immune therapeutic agent and the AKT inhibitor are administered simultaneously and the WEE1 inhibitor is administered sequentially with respect to the anti-cancer immune therapeutic agent and the AKT inhibitor; 2) the anti-cancer immune therapeutic agent and the WEE1 inhibitor are administered simultaneously and the AKT inhibitor is administered sequentially with respect to the anti-cancer immune therapeutic agent and the WEE1 inhibitor; 3) the WEE1 inhibitor and the AKT inhibitor are administered simultaneously and the anti-cancer immune therapeutic agent is administered sequentially with respect to the WEE1 inhibitor and the AKT inhibitor; or wherein all of: the anti-cancer immune therapeutic agent, the AKT inhibitor, and the WEE1 inhibitor, are administered sequentially.

31.-68. (canceled)
